(19) United States
(12) Patent Application Publication
(10) Pub. No.: US 2008/0318837 A1
(43) Pub. Date: Dec. 25, 2008

(54) Pharmaceutical Formation For Increased Epithelial Permeability of Glucose-Regulating Peptide

(75) Inventors: Steven C. Quay, Seattle, WA (US); Henry R. Costantino, Woodinville, WA (US); Michael V. Templin, Bothell, WA (US); Alexis Kays Leonard, Maple Valley, WA (US); Mary S. Kleppe, Snohomish, WA (US); Joshua O. Sestak, Kirkland, WA (US)

Correspondence Address: BROOKS KUSHMAN P.C., 1000 TOWN CENTER, TWENTY-SECOND FLOOR, SOUTHFIELD, MI, 48075, US

(73) Assignee: NASTECH PHARMACEUTICAL COMPANY INC., Bothell, WA (US)

(21) Appl. No.: 12/095,801

(22) PCT Filed: Dec. 1, 2006
(86) PCT No.: PCT/US06/61503

§ 371 Date: Jun. 2, 2008

Related U.S. Application Data
(63) Continuation-in-part of application No. 11/293,676, filed on Dec. 2, 2005.
Application No. 11/293,676 is a continuation-in-part of application No. 10/991,597, filed on Nov. 18, 2004.
(60) Provisional application No. 60/776,464, filed on Feb. 24, 2006.
(60) Provisional application No. 60/804,406, filed on Jun. 9, 2006.
(60) Provisional application No. 60/804,543, filed on Jun. 12, 2006.
(60) Provisional application No. 60/805,191, filed on Jun. 19, 2006.
(60) Provisional application No. 60/532,337, filed on Dec. 26, 2003.

(51) Int. Cl. A61K 38/00

(52) U.S. Cl. 514/2

(57) ABSTRACT

What is described is a pharmaceutical formulation comprising a mixture of a pharmaceutically effective amount of glucose-regulating peptide (GRP) and enhancers, wherein the pharmaceutical formulation is used in the treatment of a metabolic syndrome.

### BACKGROUND OF THE INVENTION

Glucose-regulating peptides (“GRP”) are a class of peptides that have therapeutic potential in the treatment of insulin dependent diabetes mellitus (IDDM), gestational diabetes or non insulin-dependent diabetes mellitus (NIDDM), the treatment of obesity, and the treatment of dyslipidemia. See U.S. Pat. No. 6,506,724, U.S. Patent Application Publication No. 20030036504A1; European Patent No. EP1083924B1; International Patent Application Publication No. WO 98/30231A1; and International Patent Application No. WO 00/73331A2. These GRPs include glucagon-like peptide (GLP), e.g., GLP-1; the exendins, especially exendin-4, also known as exenatide; and amylin peptides and amylin analogs, such as pramlintide. However, to date these GRP have only been administered to humans by injection.

A major disadvantage of drug administration by injection is that trained personnel are often required to administer the drug. Additionally, trained personal are put in harms way when administering a drug by injection. For self-administered drugs, many patients are reluctant or unable to give themselves injections on a regular basis. Injection is also associated with increased risks of infection. Other disadvantages of drug injection include variability of delivery results between individuals, as well as unpredictable intensity and duration of drug action.

Oral administration is available as an alternative; however, certain therapeutic agents exhibit very low bioavailability and considerable time delay in action when given by this route due to hepatic first-pass metabolism and degradation in the gastrointestinal tract. Thus, there is a need to develop modes of administration for GRP other than by injection and/or oral administration.

Mucosal administration of therapeutic compounds offers certain advantages over injection and other modes of administration, for example convenience and speed of delivery, as well as reducing or eliminating compliance problems and side effects that attend delivery. However, mucosal delivery of biologically active agents is limited by mucosal barrier functions and other factors. Epithelial cells make up the mucosal barrier and provide a crucial interface between the external environment and mucosal and submucosal tissues and extracellular compartments. One of the most important functions of mucosal epithelial cells is to determine and regulate mucosal permeability. In this context, epithelial cells create selective permeability barriers between different physiological compartments. Selective permeability is the result of regulated transport of molecules through the cytoplasm (the transcellular pathway) and the regulated permeability of the spaces between the cells (the paracellular pathway).

Intercellular junctions between epithelial cells are known to be involved in both the maintenance and regulation of the epithelial barrier function, and cell-cell adhesion. Tight junctions (TJ) of epithelial and endothelial cells are particularly important for cell-cell junctions that regulate permeability of the paracellular pathway, and also divide the cell surface into apical and basolateral compartments. Tight junctions form continuous circumferential intercellular contacts between epithelial cells and create a regulated barrier to the paracellular movement of water, solutes, and immune cells. They also provide a second type of barrier that contributes to cell polarity by limiting exchange of membrane lipids between the apical and basolateral membrane domains.

In the context of drug delivery, the ability of drugs to permeate epithelial cell layers of mucosal surfaces, unassisted by delivery-enhancing agents, appears to be related to a number of factors, including molecular size, lipid solubility, and ionization. In general, small molecules, less than about 300-1,000 daltons, are often capable of penetrating mucosal barriers, however, as molecular size increases, permeability decreases rapidly. Transdermal drug delivery permits permeation of larger molecules through the epithelial cell layers of the skin. Transdermal administration, such as dermal patch, is another alternative delivery route for larger macromolecular drugs. However, transdermal delivery may still present more size limitations than injection. For these reasons, mucosal and epidermal drug administration typically requires larger amounts of drug than administration by injection. Other therapeutic compounds, including large molecule drugs, are often refractory to mucosal delivery. In addition to poor intrinsic permeability, large macromolecular drugs are often subject to limited diffusion, as well as lumenal and cellular enzymatic degradation and rapid clearance at mucosal sites. Thus, in order to deliver these larger molecules in therapeutically effective amounts, cell permeation enhancing agents are required to aid their passage across these mucosal and dermal surfaces and into systemic circulation where they may quickly act on the target tissue.

### BRIEF DESCRIPTION OF THE DRAWINGS

FIG. 1: Decrease in blood glucose concentration following IN administration of GLP-1 compared to Exenatide (SQ) and Saline control (IN) (corrected for endogenous glucose) in rats.

FIG. 2: Insulin response following intranasal administration of GLP-1 in rats.

FIG. 3: Gastric Emptying following intranasal administration of GLP-1 in rats.

FIG. 4: Enhanced pharmacokinetics for Exendin-4 administered IN with 2× enhancers, IN with 2× enhancers+gelatin, and IN with 1× enhancers+gelatin compared to IN control and IV in rabbits.

### DETAILED DESCRIPTION OF INVENTION

One aspect of the present invention includes the therapeutic utility of pharmaceutical formulations for the delivery of GRP, analogues of GRP, fragments of GRP, and functional derivatives of GRPs across an epithelial surface for use in the treatment of human diseases including obesity and diabetes.

The present invention fulfills foregoing needs and satisfies additional objects and advantages by providing novel, effective methods, uses, and compositions for transepithelial, especially transmucosal, delivery of GRP such as GLP and GLP analogs, amylin and amylin analogs, and exendins and exendin analogs, to treat insulin dependent diabetes mellitus (IDDM), gestational diabetes or non insulin-dependent diabetes mellitus (NIDDM), dyslipidemia, hyperglycemia, obesity, to induce satiety in an individual, and to promote weight-loss in an individual.

In exemplary embodiments, the enhanced delivery methods and compositions of the present invention provide for therapeutically effective delivery of the GRP agonist across a layer of biological cells for prevention or treatment of obesity and eating disorders in mammalian subjects. In one aspect of the invention, pharmaceutical formulations suitable for epithelial administration are provided that comprise a therapeutically effective amount of a GRP and one or more epithelial delivery-enhancing agents as described herein, which formulations are effective in an epithelial delivery method of the invention to prevent the onset or progression of obesity or eating disorders in a mammalian subject. Transepithelial delivery of a therapeutically effective amount of a GRP agonist and one or more epithelial delivery-enhancing agents yields elevated therapeutic levels of the GRP agonist in the subject.

The enhanced delivery methods and compositions of the present invention provide for therapeutically effective delivery of a GRP for prevention or treatment of a variety of diseases and conditions in mammalian subjects. GRP can be administered via a variety of epithelial routes, for example by contacting the GRP to a nasal mucosal epithelium, a bronchial or pulmonary mucosal epithelium, the oral buccal surface, the oral and small intestinal mucosal surface, or a epidermal surface. In exemplary embodiments, the methods and compositions are directed to or formulated for intranasal delivery (e.g., nasal mucosal delivery or intranasal mucosal delivery).

The foregoing GRP formulations and preparative and delivery methods of the invention provide improved epithelial delivery of a GRP to mammalian subjects. These compositions and methods can involve combinatorial formulation or coordinate administration of one or more GRPs with one or more epithelial delivery-enhancing agents. Among the epithelial delivery-enhancing agents to be selected from to achieve these formulations and methods are (A) solubilization agents; (B) charge modifying agents; (C) pH control agents; (D) degradative enzyme inhibitors; (E) mucolytic or mucus clearing agents; (F) ciliostatic agents; (G) membrane penetration-enhancing agents (e.g., (i) a surfactant, (ii) a bile salt, (iii) a phospholipid or fatty acid additive, mixed micelle, liposome, or carrier, (iv) an alcohol, (v) an enamine, (iv) an NO donor compound, (vii) a long-chain amphipathic molecule (viii) a small hydrophobic penetration enhancer; (ix) sodium or a salicylic acid derivative; (x) a glycerol ester of acetoacetic acid (xi) a cyclodextrin or beta-cyclodextrin derivative, (xii) a medium-chain fatty acid, (xiii) a chelating agent, (xiv) an amino acid or salt thereof, (xv) an N-acetylamino acid or salt thereof, (xvi) an enzyme degradative to a selected membrane component, (xvii) an inhibitor of fatty acid synthesis, (xviii) an inhibitor of cholesterol synthesis; or (xiv) any combination of the membrane penetration enhancing agents of (i)-(xviii)); (H) modulatory agents of epithelial junction physiology, such as nitric oxide (NO) stimulators, chitosan, and chitosan derivatives; (I) vasodilator agents; (J) selective transport-enhancing agents; and (K) stabilizing delivery vehicles, carriers, supports or complex-forming species with which the GRP (s) is/are effectively combined, associated, contained, encapsulated or bound to stabilize the active agent for enhanced epithelial delivery.

In various embodiments of the invention, a GRP is combined with one, two, three, four or more of the epithelial delivery-enhancing agents recited in (A)-(K), above. These epithelial delivery-enhancing agents may be admixed, alone or together, with the GRP, or otherwise combined therewith in a pharmaceutically acceptable formulation or delivery vehicle. Formulation of a GRP with one or more of the epithelial delivery-enhancing agents according to the teachings herein (optionally including any combination of two or more epithelial delivery-enhancing agents selected from (A)-(K) above) provides for increased bioavailability of the glucose-regulating binding peptide following delivery thereof to an epithelial surface of a mammalian subject. In addition to adding epithelial delivery-enhancing agents to the formulation, modification of the GRP, such as through the addition of a hydrophobic group may be used to effect bioavailability of the peptide.

Thus, the present invention is a method for suppressing appetite, promoting weight loss, decreasing food intake, or treating obesity and/or diabetes in a mammal comprising transepithelially administering a formulation comprised of a GRP.

The present invention further provides for the use of a GRP for the production of medicament for the transepithelial administration of a GRP for treating hyperglycemia, diabetes mellitus, metabolic syndrome, dyslipidemia, suppressing appetite, promoting weight loss, decreasing food intake, or treating obesity in a mammal.

A mucosally effective dose of GRP within the pharmaceutical formulations of the present invention comprises, for example, between about 0.001 pmol to about 100 pmol per kg body weight, between about 0.01 pmol to about 10 pmol per kg body weight, or between about 0.1 pmol to about 5 pmol per kg body weight. In further exemplary embodiments, dosage of GRP is between about 0.5 pmol to about 1.0 pmol per kg body weight. In a preferred embodiment an intranasal dose will range from 0.1-100 μg/kg, or about 7-7000 μg, more preferably 0.5-30 μg/kg, or 35 to 2100 μg. More specific doses of the intranasal GRP include 20 μg, 50 μg, 100 μg, 150 μg, 200 μg to 400 μg, 500 μg, 800 to 1000 μg and 1200 to 1800 μg. The pharmaceutical formulations of the present invention may be administered one or more times per day, or 3 times per week or once per week for between one week and at least 96 weeks or even for the life of the individual patient or subject. In certain embodiments, the pharmaceutical formulations of the invention are administered one or more times daily, two times daily, four times daily, six times daily, or eight times daily.

Epithelial delivery-enhancing agents are employed which enhance delivery of GRP into or across a cellular layer, including a nasal mucosal surface. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The epithelial delivery-enhancing agent of the present invention may be a pH control agent. The pH of the pharmaceutical formulation of the present invention is a factor affecting absorption of GRP via paracellular and transcellular pathways to drug transport. In one embodiment, the pharmaceutical formulation of the present invention is pH adjusted to between about pH 2 to 8. In a further embodiment, the pharmaceutical formulation of the present invention is pH adjusted to between about pH 3.0 to 6.0. In a further embodiment, the pharmaceutical formulation of the present invention is pH adjusted to between about pH 3.5 to 5.5. Generally, the pH is 4.5±0.5.

As noted above, the present invention provides improved methods and compositions for epithelial delivery of GRP to mammalian subjects for treatment or prevention of a variety of diseases and conditions. Examples of appropriate mammalian subjects for treatment and prophylaxis according to the methods of the invention include, but are not restricted to, humans and non-human primates, livestock species, such as horses, cattle, sheep, and goats, and research and domestic species, including dogs, cats, mice, rats, guinea pigs, and rabbits.

In order to provide better understanding of the present invention, the following definitions are provided:

### Glucagon-Like Peptides (GLP)

Included within the invention are analogues, fragments, mimetics, and functional derivatives of GLP proteins and peptides. Incretins are gut derived hormones that stimulate insulin secretion in response to nutrient intake (in a glucose-dependent fashion). Two naturally occurring incretins include glucose-dependent insulinotropic peptide (GIP) and glucagons like peptide-1 (GLP-1). GLP-1 is released from the cells in the gut in response to food. GLP-1 binds to GLP-1 receptors on beta cells of the pancreas, stimulating the release of insulin. GLP-1[7-36]NH₂, also known as proglucagon[78-107] and most commonly as “GLP-1,” has an insulinotropic effect, stimulating insulin secretion; GLP-1 also inhibits glucagon secretion [Orskov, et al., Diabetes 42:658-61, 1993; D'Alessio, et al., J. Clin. Invest. 97:133-38, 1996]. GLP-1 is reported to inhibit gastric emptying [Williams B., et al., J. Clin. Encocrinol Metab. 81:(1):327-32, 1996; Wettergren A., et al., Dig. Dis. Sci. 38:(4):665-73, 1993], and gastric acid secretion. [Schjoldager B. T., et al., Dig. Dis. Sci. 34(5):703-8, 1989; O'Halloran D. J., et al., J. Endocrinol. 126(1):169-73, 1990; Wettergren A., et al., Dig. Dis. Sci. 38:(4):665-73, 1993]. GLP-1 [7-37], which has an additional glycine residue at its carboxy terminus, also stimulates insulin secretion in humans [Orskov, et al., Diabetes 42:658-61, 1993]. A transmembrane G-protein adenylate-cyclase-coupled receptor believed to be responsible for the insulinotropic effect of GLP-1 is reported to have been cloned from a .beta.-cell line [Thorens, Proc. Natl. Acad. Sci. USA 89:8641-45, 1992].

A major limitation of GLP-1 for therapeutics is its rapid degradation by the ubiquitous enzyme dipeptidyl peptidase-IV (DPP-IV). DPP-IV inhibitors (LAF237; MK-0431) have been used to improve the duration of endogenous GLP-1 activity. U.S. Food and Drug Administration (FDA) approved JANUVIA™ (sitagliptin phosphate), Merck & Co., Inc., an oral DPP-IV inhibitor available in the United States for the treatment of type 2 diabetes. A method for the treatment of metabolic diseases in a mammal comprising co-administration of a compound capable of binding to a secondary binding site of DPP-IV and DPP-IV like enzymes and at least one anti-diabetic agent was described in U.S. Patent Application No. 20060234940.

Incretin mimetics are a class of drugs that mimic the anidiabetic or glucose-lowering actions of naturally occurring human incretin hormones like GLP-1. The actions of incretin mimetics include stimulating the body's ability to produce insulin in response to elevated blood sugar levels, inhibiting the release of glucagon hormone, slowing nutrient absorption into the bloodstream, slowing the rate of gastric emptying, promoting satiety and reducing food intake. Incretin mimetics were developed for use in the treatment of type 2 diabetes and include the following: GLP-1 derivatives (Liraglutide and CJC-1131) and Exenatide. CJC-1131 (ConjuChem, Montreal, Canada) has a reactive linker that allows covalent binding to serum albumin resulting in increased resistance to DPP-IV degradation. Liraglutide (Novo Nordisk, Copenhagen, Denmark) is a GLP-1 derivative designed to overcome the effects of DPP-IV degradation via acylation with a fatty acid chain. The structure of Liraglutide is shown in WHO Drug Information, Vol. 17, No. 2 (2003).

The invention includes modifications of GRPs by attachment of a hydrophobic group, such as fatty acids, to the peptide. Further examples of modified derivatives of GLP-1 with desirable pharmacokinetic properties are described in Knudsen et al., J. Med. Chem. 43:1664-1669, 2000, and are hereby incorporated by reference. These GLP-1 compounds were derivatized with fatty acids in order to protract their action by facilitating binding to serum albumin. The following parent peptides and acyl substitutions were described: K⁸R^{26,34}-GLP-1(7-37) (K⁸: γ-Glu-C16); K¹⁸R^{26,34}-GLP-1 (7-37) (K¹⁸: γ-Glu-C16); K²³R^{26,34}-GLP-1(7-37) (K²³: γ-Glu-C16); R³⁴-GLP-1(7-37) (K²⁶: γ-Glu-C16); K²⁷R^{26,34}-GLP-1(7-37) (K²⁷:γ-Glu-C16); R²⁶-GLP-1(7-37) (K³⁴:γ-Glu-C16); K³⁶R^{26,34}-GLP-1(7-37) (K³⁶: γ-Glu-C16); R^{26,34}-GLP-1(7-38) (K³⁸:γ-Glu-C16); GLP-1(7-37) (K^{26,34}:bis-C16-diacid); GLP-1(7-37) (K^{26,34}:bis-γ-Glu-C16); GLP-1(7-37) (K^{26,34}:bis-γ-Glu-C14; GLP-1(7-37) (K^{26,34}:bis-C12-diacid); R³⁴-GLP-1(7-37) (K²⁶:C16-diacid); R³⁴GLP-1(7-37) (K²⁶:C14-diacid); R³⁴-GLP-1(7-37) K²⁶: γ-Glu-C18); R³⁴-GLP-1(7-37) (K²⁶:γ-Glu-C14); R³⁴-GLP-1(7-37) (K²⁶:γ-Glu-C12); desamino-H⁷R³⁴-GLP-1(7-37) (K²⁶:γ-Glu-C16); R³⁴-GLP-1(7-37) (K²⁶:GABA-C16); R³⁴-GLP-1(7-37) (K²⁶:β-Ala-C16); R³⁴-GLP-1(7-37) (K²⁶:Iso-Nip-C16); desamino-H⁷R²⁶-GLP-1(7-37) (K³⁴:γ-Glu-C16); desamino-H⁷R²⁶-GLP-1(7-37) (K³⁴:C8); desamino-H⁷R²⁶-GLP-1(7-37) (K³⁴:γ-Glu-C8); K^{36,34}-GLP-1(7-36) (K³⁶:C20-diacid); K^{36,34}-GLP-1(7-36)(36:C16-diacid); K^{36,34}-GLP-1(7-36) (K³⁶: γ-Glu-C18); R^{26,34}-GLP-1(7-38) (K³⁸:C16-diacid); R^{26,34}-GLP-1(7-38) (K³⁸:C12-diacid); R^{26,34}-GLP-1(7-38) (K³⁸:γ-Glu-C18); R^{26,34}-GLP-1(7-38) (K³⁸:γ-Glu-C14); C⁸R^{26,34}-GLP-1(7-38) (K³⁸:γ-Glu-C16); E³⁷R^{26,34}-GLP-1(7-38) (K³⁸:γ-Glu-C16); E³⁷C⁸R^{26,34}-GLP-1(7-38) (K³⁸:γ-Glu-C16); and E³⁷C⁸R^{26,34}-GLP-1(7-38) (K³⁸:γ-Glu-C18).

The amino acid sequence of GLP-1 is given i.a. by Schmidt, et al., Diabetologia 28:704-707, 1985. Human GLP-1 is a 37 amino acid residue peptide originating from preproglucagon which is synthesized, i.a. in the L-cells in the distal ileum, in the pancreas and in the brain. Processing of preproglucagon to GLP-1(7-36)amide, GLP-1(7-37) and GLP-2 occurs mainly in the L-cells. Although the interesting pharmacological properties of GLP-1(7-37) and analogues thereof have attracted much attention in recent years only little is known about the structure of these molecules. The secondary structure of GLP-1 in micelles has been described by Thorton, et al., Biochemistry 33:3532-3539, 1994), but in normal solution, GLP-1 is considered a very flexible molecule.

GLP-1 and analogues of GLP-1 and fragments thereof are useful i.a. in the treatment of Type 1 and Type 2 diabetes and obesity. GLP-1 analogues, GLP-1 fragments and functional derivatives of GLP-1 described in Holst, J., Expert Opin. Emerg. Drugs 9(1):155-161, 2004; Rolin, R., et al., Am. J. Physiol. Endocrinol. Metab. 283:E745-E752, 2002; Deacon, C., Diabetes 53:2181-2187, 2004; Perry, T., et al., Trends Pharmacol. Sci. 24(7):377-383, 2003; Holz, G., et al., Curr. Med. Chem. 10(22):2471-2481, 2003; Naslund, E., et al., Regul. Pept. 106:89-95, 2002; patent applications WO 87/06941; WO 90/11296; WO 91/11457; patents EP 0708179-A2 and EP 0699686-A2 are incorporated by reference herein in their entirety:

WO 87/06941 discloses GLP-1 fragments, including GLP-1(7-37), and functional derivatives thereof and to their use as an insulinotropic agent.

WO 90/11296 discloses GLP-1 fragments, including GLP-1 (7-36), and functional derivatives thereof which have an insulinotropic activity which exceeds the insulinotropic activity of GLP-1(1-36) or GLP-1 (1-37) and to their use as insulinotropic agents.

WO 91/11457 discloses analogues of the active GLP-1 peptides 7-34, 7-35, 7-36, and 7-37 which can also be useful as GLP-1 moieties.

EP 0708179-A2 (Eli Lilly & Co.) discloses GLP-1 analogues and derivatives that include an N-terminal imidazole group and optionally an unbranched C₆-C₁₀ acyl group in attached to the lysine residue in position 34.

EP 0699686-A2 (Eli Lilly & Co.) discloses certain N-terminal truncated fragments of GLP-1 that are reported to be biologically active.

The amino acid sequence of GLP-1 (1-37) is:

(SEQ ID NO: 1)

HDEFERHAEGTFTSDVSSYLEGQAAKEFIAWLVKGRG.

The amino acid sequence of GLP-1 (7-37) is:

| | |
|---|---|
| HAEGTFTSDVSSYLEGQAAKEFIAWLVKGRG. | (SEQ ID NO: 2) |

The amino acid sequence of GLP-1 (7-36) is:

| | |
|---|---|
| HAEGTFTSDVSSYLEGQAAKEFIAWLVKGR. | (SEQ ID NO: 3) |

The amino acid sequence of GLP-1 (7-34) is:

| | |
|---|---|
| HAEGTFTSDVSSYLEGQAAKEFIAWLVK. | (SEQ ID NO: 4) |

The amino acid sequence of GLP-1 (9-36) is:

| | |
|---|---|
| EGTFTSDVSSYLEGQAAKEFIAWLVKGR. | (SEQ ID NO: 5) |

The GLP-1 analogs listed below have enhanced DPP-IV resistance.

The amino acid sequence of the GLP-1 analog GG is:

| | |
|---|---|
| HGEGTFTSDVSSYLEGQAAKEFIAWLVKGR. | (SEQ ID NO: 6) |

The amino acid sequence of the GLP-1 analog GG₁ is:

| | |
|---|---|
| HGEGTFTSDVSSYLEGQAAKEFIAWLVKGRPSS. | (SEQ ID NO: 7) |

The amino acid sequence of the GLP-1 analog GG₂ is:

(SEQ ID NO: 8)

HGEGTFTSDVSSYLEGQAAKEFIAWLVKGRPSSGAP.

The amino acid sequence of the GLP-1 analog GG₃ is:

(SEQ ID NO: 9)

HGEGTFTSDVSSYLEGQAAKEFIAWLVKGRPSSGAPPPS.

The amino acid sequence of the GLP-1 analog GLP-1 ET is:

(SEQ ID NO: 10)

HAEGTFTSDVSSYLEGQAAKEFIAWLVKGGPSSGAPPPS.

The amino acid sequence of the GLP-1 synthetic analog NN2211 is: HAEGTFTSDVSSYLEGQAAK*EFIAWLVRGRG (SEQ ID NO: 11) where K* at position 26 of the amino acid chain is modified by acylation to generate a hexadecanoyl side chain (i.e., K-N-ε-(γ-Glu (N-ε-hexadecanoyl).

The amino acid sequence of the GLP-1 synthetic analog CJC-1131 is: HA*EGTFTSDVSSYLEGQAAKEFIAWLVKGRK (SEQ ID NO: 12) where A* at position 8 of the amino acid chain is a D-alanine substituted for a L-alanine and the K* at position 37 of the amino acid chain has a [2-[2-[2-maleimidopropionamido(ethoxy)ethoxy]acetamide linker at its ε amino group.

The amino acid sequence of the GLP-1 synthetic analog LY315902 is: des-HAEGTFTSDVSSYLEGQAAREFIAWLVK*GRG (SEQ ID NO: 13) where the histidine residue at the N-terminus (des-H) does not contain an amino group and the K* at position 34 is modified by acylation to generate a octanoyl side chain (i.e., K-(octoanoyl)).

According to the present invention GLP-1 also include the free bases, acid addition salts or metal salts, such as potassium or sodium salts of the peptides, and GLP-1 peptides that have been modified by such processes as amidation, glycosylation, acylation, sulfation, phosphorylation, acetylation, cyclization and other well known covalent modification methods.

### Exendins and Exendin Agonists

Included within the invention are analogues, fragments, and functional derivatives of exendin proteins and peptides. Exendins are peptides that were first isolated from the salivary secretions of the Gila-monster, a lizard found in Arizona, and the Mexican Beaded Lizard. Exendin-3 is present in the salivary secretions of Heloderma horridum, and exendin-4 is present in the salivary secretions of Heloderma suspectum [Eng, J., et al., J. Biol. Chem. 265:20259-62, 1990; Eng., J., et al., J. Biol. Chem. 267:7402-05, 1992]. The exendins have some sequence similarity to several members of the glucagon-like peptide family, with the highest homology, 53%, being to the incretin hormone GLP-1[7-36]NH₂ [Goke, et al., J. Biol. Chem. 268:19650-55, 1993].

The generic name for synthetic exendin-4 is exenatide [WHO Drug Information, Vol. 18, No. 1, 2004]. Exenatide is a synthetic Exendin-4. Exenatide mirrors the effects of GLP-1, but is more potent because of its resistant to DPP-IV degradation. BYETTA® is the commercially available version of exenatide (Amylin & Lilly). The U.S. FDA approved BYETTA (Exenatide) injection as an adjunctive therapy to type 2 diabetes where oral metformin and/or sulfonylurea treatment are not adequate to achieve glycemic control. In addition to improved glycemic control, subjects in the studies using exenatide also experienced weight loss.

The present invention is directed to novel methods for treating diabetes and conditions that would be benefited by lowering plasma glucose or delaying and/or slowing gastric emptying or inhibiting food intake comprising the intranasal administration of an exendin, an exendin analog, an exendin agonist, a modified exendin, a modified exendin analog, or a modified exendin agonist, or any combinations thereof, for example:

Exendin-3:

(SEQ ID NO: 14)

His Ser Asp Gly Thr Phe Thr Ser Asp Leu Ser Lys
Gln Met Glu Glu Glu Ala Val Arg Leu Phe Ile Glu
Trp Leu Lys Asn Gly Gly Pro Ser Ser Gly Ala Pro
Pro Pro Ser,
or,
exendin-4 (synthetic exendin-4 (exenatide)):

(SEQ ID NO: 15)

His Gly Glu Gly Thr Phe Thr Ser Asp Leu Ser Lys
Gln Met Glu Glu Glu Ala Val Arg Leu Phe Ile Glu
Trp Leu Lys Asn Gly Gly Pro Ser Ser Gly Ala Pro
Pro Pro Ser
wherein the C-terminus serine is amidated,
or
insulinotropic fragments of exendin-4:
Exendin-4(1-31)

(SEQ ID NO: 16)

His Gly Glu Gly Thr Phe Thr Ser Asp Leu Ser Lys
Gln Met Glu Glu Glu Ala Val Arg Leu Phe Ile Glu
Trp Leu Lys Asn Gly Gly Pro;
y.sup.31 Exendin-4(1-31)

(SEQ ID NO: 17)

His Gly Glu Gly Thr Phe Thr Ser Asp Leu Ser Lys
Gln Met Glu Glu Glu Ala Val Arg Leu Phe Ile Glu
Trp Leu Lys Asn Gly Gly Tyr,
or
inhibitory fragments of exendin-4:
Exendin-4(9-39)

(SEQ ID NO: 18)

Asp Leu Ser Lys Gln Met Glu Glu Gln Ala Val Arg
Leu Phe Ile Glu Trp Leu Lys Asn Gly Gly Pro Ser
Ser Gly Ala Pro Pro Pro Ser,
or
other preferred exendin agonists:
exendin-4 (1-30)

(SEQ ID NO: 19)

His Gly Gln Gly Thr Phe Thr Ser Asp Leu Ser Lys
Gln Met Gln Glu Glu Ala Val Arg Leu Phe Ile Glu
Trp Leu Lys Asn Gly Gly,
exendin-4 (1-30) amide

(SEQ ID NO: 20)

His Gly Glu Gly Thr Phe Thr Ser Asp Leu Ser Lys
Gln Met Glu Glu Gln Ala Val Arg Len Phe Ile Gln
Trp Leu Lys Asn Gly Gly-NH.snb.2,
exendin-4 (1-28) amide

(SEQ ID NO: 21)

His Gly Glu Gly Thr Phe Thr Ser Asp Leu Ser Lys
Gln Met Glu Glu Gln Ala Val Arg Leu Phe Ile Glu
Trp Leu Lys Asn-NH.sub.2,
.snp. 14 Leu,.snp.25 Phe exendin-4 amide

(SEQ ID NO: 22)

His Gly Glu Gly Thr Phe Thr Ser Asp Len Ser Lys
Gln Len Glu Gln Gln Ala Val Arg Len Phe Ile Gln
Phe Len Lys Asn Gly Gly Pro Ser Ser Gly Ala Pro
Pro Pro Ser-NH.snb.2,
.snp. 14 Len, .snp.25 Phe exendin-4 (1-28) amide

(SEQ ID NO: 23)

His Gly Gln Gly Thr Phe Thr Ser Asp Len Ser Lys
Gln Leu Gln Gln Gln Ala Val Arg Leu Phe Ile Gln
Phe Leu Lys Asn-NH.snb.2,
and
.snp.14 Leu, .snp.22 Ala, .snp.25 Phe exendin-4
(1-28) amide

(SEQ ID NO: 24)

His Gly Gln Gly Thr Phe Thr Ser Asp Leu Ser Lys
Gln Leu Gln Gln Gln Ala Val Arg Leu Ala Ile Gln
Phe Leu Lys Asn-NH.snb.2.

or sequences incorporated by reference that have been disclosed in U.S. Pat. No. 5,424,286; U.S. Pat. No. 6,506,724; U.S. Pat. No. 6,528,486; U.S. Pat. No. 6,593,295; U.S. Pat. No. 6,872,700; U.S. Pat. No. 6,902,744; U.S. Pat. No. 6,924,264; and U.S. Pat. No. 6,956,026,
   or other compounds which effectively bind to the receptor at which exendin exerts its actions which are beneficial in the treatment of diabetes and conditions that would be benefited by lowering plasma glucose or delaying and/or slowing gastric emptying or inhibiting food intake. The use of exendin-3 and exendin-4 as insulinotrophic agents for the treatment of diabetes mellitus and the prevention of hyperglycemia was disclosed in U.S. Pat. No. 5,424,286. Exendins have also been shown to be useful in the modulation of triglyceride levels and to treat dyslipidemia.

Thus the invention provides for the peptides or peptide fragments, made synthetically or purified from natural sources, which embody the biological activity of the exendins or fragments thereof, as described by the present specification.

According to the present invention exendins also include the free bases, acid addition salts or metal salts, such as potassium or sodium salts of the peptides, and exendin peptides that have been modified by such processes as amidation, glycosylation, acylation, sulfation, phosphorylation, acetylation, cyclization and other well known covalent modification methods.

Thus, according to the present invention, the above-described peptides are incorporated into formulations suitable for transepithelial delivery, especially intranasal and dermal delivery.

### Biological Membranes

“Biological membrane” is defined as membrane material present within a living organism, preferably an animal, more preferably a human, that separates one area of the organism from another. In many instances, the biolocial membrane separates the organism with its outer surroundings or environment. Non-limiting examples of biological membrane include the mucus and skin membranes in a human being.

### Epithelial Delivery Enhancing Agents

“Epithelial delivery enhancing agents” are defined as chemicals and other excipients that, when added to a formulation comprising water, salts and/or common buffers and GRP (the control formulation) produce a formulation that produces a significant increase in transport of GRP across a biological membrane as measured by the maximum blood, serum, or cerebral spinal fluid concentration (Cₘₐₓ) or by the area under the curve, AUC, in a plot of concentration versus time. Epithelial biological membranes may include the nasal, oral, intestinal, buccal, bronchopulmonary, vaginal, rectal, and dermal surfaces. Transepithelial delivery enhancing agents are sometimes called carriers.

### Mucosal Delivery Enhancing Agents

“Mucosal delivery enhancing agents” are defined as chemicals and other excipients that, when added to a formulation comprising water, salts and/or common buffers and GRP (the control formulation) produce a formulation that produces a significant increase in transport of GRP across a mucosa as measured by the maximum blood, serum, or cerebral spinal fluid concentration (Cₘₐₓ) or by the area under the curve, AUC, in a plot of concentration versus time. A mucosa includes the nasal, oral, intestinal, buccal, bronchopulmonary, vaginal, and rectal mucosal surfaces and in fact includes all mucus-secreting membranes lining all body cavities or passages that communicate with the exterior. Mucosal delivery enhancing agents are sometimes called carriers.

### Endotoxin-Free Formulation

“Endotoxin-free formulation” means a formulation which contains a GRP and one or more epithelial delivery enhancing agents that is substantially free of endotoxins and/or related pyrogenic substances. Endotoxins include toxins that are confined inside a microorganism and are released only when the microorganisms are broken down or die. Pyrogenic substances include fever-inducing, thermostable substances (glycoproteins) from the outer membrane of bacteria and other microorganisms. Both of these substances can cause fever, hypotension and shock if administered to humans. Producing formulations that are endotoxin-free can require special equipment, expert artisans, and can be significantly more expensive than making formulations that are not endotoxin-free. Because intravenous administration of GLP or amylin simultaneously with infusion of endotoxin in rodents has been shown to prevent the hypotension and even death associated with the administration of endotoxin alone (U.S. Pat. No. 4,839,343), producing endotoxin-free formulations of these therapeutic agents would not be expected to be necessary for non-parental (non-injected) administration.

### Non-Infused Administration

“Non-infused administration” means any method of delivery that does not involve an injection directly into an artery or vein, a method which forces or drives (typically a fluid) into something and especially to introduce into a body part by means of a needle, syringe or other invasive method. Non-infused administration includes subcutaneous injection, intramuscular injection, intraparitoneal injection and the non-injection methods of delivery to a biological membrane.

### Methods and Compositions of Delivery

Improved methods and compositions for epithelial administration of GRP to mammalian subjects optimize GRP dosing schedules. The present invention provides epithelial delivery of GRP formulated with one or more epithelial delivery-enhancing agents wherein GRP dosage release is substantially normalized and/or sustained for an effective delivery period of GRP release ranges from approximately 0.1 to 2.0 hours; 0.4 to 1.5 hours; 0.7 to 1.5 hours; or 0.8 to 1.0 hours; following epithelial administration. The sustained release of GRP achieved may be facilitated by repeated administration of exogenous GRP utilizing methods and compositions of the present invention.

### Compositions and Methods of Sustained Release

Improved compositions and methods for epithelial administration of GRP to mammalian subjects optimize GRP dosing schedules. The present invention provides improved epithelial (e.g., nasal) delivery of a formulation comprising GRP in combination with one or more epithelial delivery-enhancing agents and an optional sustained release-enhancing agent or agents. Epithelial delivery-enhancing agents of the present invention yield an effective increase in delivery, e.g., an increase in the maximal plasma concentration (Cₘₐₓ) to enhance the therapeutic activity of epithelially-administered GRP. A second factor affecting therapeutic activity of GRP in the blood plasma and CNS is residence time (RT). Sustained release-enhancing agents, in combination with intranasal delivery-enhancing agents, increase Cₘₐₓ and increase residence time (RT) of GRP. Polymeric delivery vehicles and other agents and methods of the present invention that yield sustained release-enhancing formulations, for example, polyethylene glycol (PEG), are disclosed herein. The present invention provides an improved GRP delivery method and dosage form for treatment of symptoms related to obesity, diabetes, hyperglycemia, metabolic syndrome, coronary syndrome, colon cancer, exendin cancer, breast cancer, myocardial infraction, promoting neurogenesis, suppressing appetite, promoting weight loss, and decreasing food intake in mammalian subjects.

Within the epithelial delivery formulations and methods of the invention, the GRP is frequently combined or coordinately administered with a suitable carrier or vehicle for epithelial delivery. As used herein, the term “carrier” means pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories can be found in the U.S. Pharmacopeia National Formulary, 1857-1859, 1990. Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. The amount of active ingredient that can be combined with the carrier materials to produce a single dosage form will vary depending upon the particular mode of administration.

Within the epithelial delivery compositions and methods of the invention, various delivery-enhancing agents are employed which enhance delivery of GRP into or across a cellular layer. In this regard, delivery of GRP across the epithelium can occur “transcellularly” or “paracellularly.” The extent to which these pathways contribute to the overall flux and bioavailability of the GRP depends upon the environment of the biological membrane, the physico-chemical properties the active agent, and the properties of the epithelium. Paracellular transport involves only passive diffusion, whereas transcellular transport can occur by passive, facilitated or active processes. Generally, hydrophilic, passively transported, polar solutes diffuse through the paracellular route, while more lipophilic solutes use the transcellular route. Absorption and bioavailability (e.g., as reflected by a permeability coefficient or physiological assay), for diverse, passively and actively absorbed solutes, can be readily evaluated, in terms of both paracellular and transcellular delivery components, for any selected GRP within the invention. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The paracellular route represents a relatively small fraction of accessible surface area of the nasal mucosal epithelium. In general terms, it has been reported that cell membranes occupy a mucosal surface area that is a thousand times greater than the area occupied by the paracellular spaces. Thus, the smaller accessible area, and the size- and charge-based discrimination against macromolecular permeation would suggest that the paracellular route would be a generally less favorable route than transcellular delivery for drug transport. Surprisingly, the methods and compositions of the invention provide for significantly enhanced transport of biotherapeutics into and across mucosal epithelia via the paracellular route. Therefore, the methods and compositions of the invention successfully target both paracellular and transcellular routes, alternatively or within a single method or composition.

As used herein, epithelial delivery-enhancing agents include agents which enhance the release or solubility (e.g., from a formulation delivery vehicle), diffusion rate, penetration capacity and timing, uptake, residence time, stability, effective half-life, peak or sustained concentration levels, clearance and other desired epithelial delivery characteristics (e.g., as measured at the site of delivery, or at a selected target site of activity such as the bloodstream or central nervous system) of GRP or other biologically active compound(s). Enhancement of epithelial delivery can thus occur by any of a variety of mechanisms, for example by increasing the diffusion, transport, persistence or stability of GRP, increasing membrane fluidity, modulating the availability or action of calcium and other ions that regulate intracellular or paracellular permeation, solubilizing membrane components (e.g., lipids), changing non-protein and protein sulfhydryl levels in mucosal tissues, increasing water flux across the cellular layer, modulating epithelial junctional physiology, reducing the viscosity of mucus overlying the mucosal epithelium, reducing mucociliary clearance rates, and other mechanisms.

As used herein, an “effective amount of GRP” contemplates effective delivery of GRP to a target site for drug activity in the subject that may involve a variety of delivery or transfer routes. For example, a given active agent may find its way through clearances between cells of the mucosa and reach an adjacent vascular wall, while by another route the agent may, either passively or actively, be taken up into mucosal cells to act within the cells or be discharged or transported out of the cells to reach a secondary target site, such as the systemic circulation. The methods and compositions of the invention may promote the translocation of active agents along one or more such alternate routes, or may act directly on the mucosal tissue or proximal vascular tissue to promote absorption or penetration of the active agent(s). The promotion of absorption or penetration in this context is not limited to these mechanisms.

As used herein “peak concentration (Cₘₐₓ) of GRP in a blood plasma”, “area under concentration vs. time curve (AUC) of GRP in a blood plasma”, “time to maximal plasma concentration (tₘₐₓ) of GRP in a blood plasma” are pharmacokinetic parameters known to one skilled in the art. Laursen, et al., Eur. J. Endocrinology 135:309-315, 1996. The “concentration vs. time curve” measures the concentration of GRP in a blood serum of a subject vs. time after administration of a dosage of GRP to the subject either by intranasal, intramuscular, subcutaneous, or other parenteral route of administration. “Cₘₐₓ,” is the maximum concentration of GRP in the blood serum of a subject following a single dosage of GRP to the subject. “tₘₐₓ,” is the time to reach maximum concentration of GRP in a blood serum of a subject following administration of a single dosage of GRP to the subject.

As used herein, “area under concentration vs. time curve (AUC) of GRP in a blood plasma” is calculated according to the linear trapezoidal rule and with addition of the residual areas. A decrease of 23% or an increase of 30% between two dosages would be detected with a probability of 90% (type II error β=10%). The “delivery rate” or “rate of absorption” is estimated by comparison of the time (tₘₐₓ) to reach the maximum concentration (Cₘₐₓ). Both Cₘₐₓ and tₘₐₓ are analyzed using non-parametric methods. Comparisons of the pharmacokinetics of intramuscular, subcutaneous, intravenous and intranasal GRP administrations were performed by analysis of variance (ANOVA). For pair wise comparisons a Bonferroni-Holmes sequential procedure is used to evaluate significance. The dose-response relationship between the three nasal doses is estimated by regression analysis. P<0.05 is considered significant. Results are given as mean values+/−SEM.

While the mechanism of absorption promotion may vary with different epithelial delivery-enhancing agents of the invention, useful reagents in this context will not substantially adversely affect the tissue and will be selected according to the physicochemical characteristics of the particular GRP or other active or delivery-enhancing agent. In this context, delivery-enhancing agents that increase penetration or permeability of mucosal tissues will often result in some alteration of the protective permeability barrier of the mucosa. For such delivery-enhancing agents to be of value within the invention, it is generally desired that any significant changes in permeability of the biological membrane be reversible within a time frame appropriate to the desired duration of drug delivery. Furthermore, there should be no substantial, cumulative toxicity, nor any permanent deleterious changes induced in the barrier properties of the biological membrane with long-term use.

Within certain aspects of the invention, absorption-promoting agents for coordinate administration or combinatorial formulation with GRP of the invention are selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium laurylsulfate, oleic acid, and the bile salts, may be employed to enhance biological membrane penetration of the GRP. In additional aspects, surfactants (e.g., polysorbates) are employed as adjunct compounds, processing agents, or formulation additives to enhance transepithelial delivery of the GRP. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the GRP from the vehicle into the biological membrane.

Additional epithelial delivery-enhancing agents that are useful within the coordinate administration and processing methods and combinatorial formulations of the invention include, but are not limited to, mixed micelles; enamines; nitric oxide donors (e.g., S-nitroso-N-acetyl-DL-penicillamine, NOR1, NOR4-which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for epithelial delivery. Other absorption-promoting agents are selected from a variety of carriers, bases and excipients that enhance epithelial delivery, stability, activity or trans-epithelial penetration of the GRP. These include, inter alia, cyclodextrins and β-cyclodextrin derivatives (e.g., 2-hydroxypropyl-β-cyclodextrin and heptakis(2,6-di-O-methyl-β-cyclodextrin). These compounds, optionally conjugated with one or more of the active ingredients and further optionally formulated in an oleaginous base, enhance bioavailability in the epithelial formulations of the invention. Yet additional absorption-enhancing agents adapted for epithelial delivery include medium-chain fatty acids, including mono- and diglycerides (e.g., sodium caprate—extracts of coconut oil, Capmul), and triglycerides (e.g., amylodextrin, Estaram 299, Miglyol 810).

The epithelial therapeutic and prophylactic compositions of the present invention may be supplemented with any suitable penetration-promoting agent that facilitates absorption, diffusion, or penetration of GRP across biological membrane barriers. The penetration promoter may be any promoter that is pharmaceutically acceptable. Thus, in more detailed aspects of the invention compositions are provided that incorporate one or more penetration-promoting agents selected from sodium salicylate and salicylic acid derivatives (acetyl salicylate, choline salicylate, salicylamide, etc.); amino acids and salts thereof (e.g., monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine etc.—inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts). Also provided as penetration-promoting agents within the methods and compositions of the invention are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Within various aspects of the invention, improved nasal mucosal delivery formulations and methods are provided that allow delivery of GRP and other therapeutic agents within the invention across biological membrane barriers between administration and selected target sites. Certain formulations are specifically adapted for a selected target cell, tissue or organ, or even a particular disease state. In other aspects, formulations and methods provide for efficient, selective endo- or transcytosis of GRP specifically routed along a defined intracellular or intercellular pathway. Typically, the GRP is efficiently loaded at effective concentration levels in a carrier or other delivery vehicle, and is delivered and maintained in a stabilized form, e.g., at the nasal mucosa and/or during passage through intracellular compartments and membranes to a remote target site for drug action (e.g., the blood stream or a defined tissue, organ, or extracellular compartment). The GRP may be provided in a delivery vehicle or otherwise modified (e.g., in the form of a prodrug), wherein release or activation of the GRP is triggered by a physiological stimulus (e.g., pH change, lysosomal enzymes, etc.) Often, the GRP is pharmacologically inactive until it reaches its target site for activity. In most cases, the GRP and other formulation components are non-toxic and non-immunogenic. In this context, carriers and other formulation components are generally selected for their ability to be rapidly degraded and excreted under physiological conditions. At the same time, formulations are chemically and physically stable in dosage form for effective storage.

### Peptide and Protein Analogs and Mimetics

Included within the definition of biologically active peptides and proteins for use within the invention are natural or synthetic, therapeutically or prophylactically active, peptides (comprised of two or more covalently linked amino acids), proteins, peptide or protein fragments, peptide or protein analogs, and chemically modified derivatives or salts of active peptides or proteins. A wide variety of useful analogs and mimetics of GRP are contemplated for use within the invention and can be produced and tested for biological activity according to known methods. Often, the peptides or proteins of GRP or other biologically active peptides or proteins for use within the invention are muteins that are readily obtainable by partial substitution, addition, or deletion of amino acids within a naturally occurring or native (e.g., wild-type, naturally occurring mutant, or allelic variant) peptide or protein sequence. Additionally, biologically active fragments of native peptides or proteins are included. Such mutant derivatives and fragments substantially retain the desired biological activity of the native peptide or proteins. In the case of peptides or proteins having carbohydrate chains, biologically active variants marked by alterations in these carbohydrate species are also included within the invention.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine. By aligning a peptide or protein analog optimally with a corresponding native peptide or protein, and by using appropriate assays, e.g., adhesion protein or receptor binding assays, to determine a selected biological activity, one can readily identify operable peptide and protein analogs for use within the methods and compositions of the invention. Operable peptide and protein analogs are typically specifically immunoreactive with antibodies raised to the corresponding native peptide or protein.

An approach for stabilizing solid protein formulations of the invention is to increase the physical stability of purified, e.g., lyophilized protein. This will inhibit aggregation via hydrophobic interactions as well as via covalent pathways that may increase as proteins unfold. Stabilizing formulations in this context often include polymer-based formulations, for example a biodegradable hydrogel formulation/delivery system. As noted above, the critical role of water in protein structure, function, and stability is well known. Typically, proteins are relatively stable in the solid state with bulk water removed. However, solid therapeutic protein formulations may become hydrated upon storage at elevated humidities or during delivery from a sustained release composition or device. The stability of proteins generally drops with increasing hydration. Water can also play a significant role in solid protein aggregation, for example, by increasing protein flexibility resulting in enhanced accessibility of reactive groups, by providing a mobile phase for reactants, and by serving as a reactant in several deleterious processes such as beta-elimination and hydrolysis.

Protein preparations containing between about 6% to 28% water are the most unstable. Below this level, the mobility of bound water and protein internal motions are low. Above this level, water mobility and protein motions approach those of full hydration. Up to a point, increased susceptibility toward solid-phase aggregation with increasing hydration has been observed in several systems. However, at higher water content, less aggregation is observed because of the dilution effect.

In accordance with these principles, an effective method for stabilizing peptides and proteins against solid-state aggregation for mucosal delivery is to control the water content in a solid formulation and maintain the water activity in the formulation at optimal levels. This level depends on the nature of the protein, but in general, proteins maintained below their “monolayer” water coverage will exhibit superior solid-state stability.

A variety of additives, diluents, bases and delivery vehicles are provided within the invention, that effectively control water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto. In some instances, the activity or physical stability of proteins can also be enhanced by various additives to aqueous solutions of the peptide or protein drugs. For example, additives, such as polyols (including sugars), amino acids, proteins such as collagen and gelatin, and various salts may be used.

Certain additives, in particular sugars and other polyols, also impart significant physical stability to dry, e.g., lyophilized proteins. These additives can also be used within the invention to protect the proteins against aggregation not only during lyophilization but also during storage in the dry state. For example sucrose and Ficoll 70 (a polymer with sucrose units) exhibit significant protection against peptide or protein aggregation during solid-phase incubation under various conditions. These additives may also enhance the stability of solid proteins embedded within polymer matrices.

Yet additional additives, for example sucrose, stabilize proteins against solid-state aggregation in humid atmospheres at elevated temperatures, as may occur in certain sustained-release formulations of the invention. Proteins such as gelatin and collagen also serve as stabilizing or bulking agents to reduce denaturation and aggregation of unstable proteins in this context. These additives can be incorporated into polymeric melt processes and compositions within the invention. For example, polypeptide microparticles can be prepared by simply lyophilizing or spray drying a solution containing various stabilizing additives described above. Sustained release of unaggregated peptides and proteins can thereby be obtained over an extended period of time.

Various additional preparative components and methods, as well as specific formulation additives, are provided herein which yield formulations for epithelial delivery of aggregation-prone peptides and proteins, wherein the peptide or protein is stabilized in a substantially pure, unaggregated form using a solubilization agent. A range of components and additives are contemplated for use within these methods and formulations. Exemplary of these solubilization agents are cyclodextrins (CDs), which selectively bind hydrophobic side chains of polypeptides. These CDs have been found to bind to hydrophobic patches of proteins in a manner that significantly inhibits aggregation. This inhibition is selective with respect to both the CD and the protein involved. Such selective inhibition of protein aggregation provides additional advantages within the intranasal delivery methods and compositions of the invention. Additional agents for use in this context include CD dimers, trimers and tetramers with varying geometries controlled by the linkers that specifically block aggregation of peptides and protein. Yet solubilization agents and methods for incorporation within the invention involve the use of peptides and peptide mimetics to selectively block protein-protein interactions. In one aspect, the specific binding of hydrophobic side chains reported for CD multimers is extended to proteins via the use of peptides and peptide mimetics that similarly block protein aggregation. A wide range of suitable methods and anti-aggregation agents are available for incorporation within the compositions and procedures of the invention.

### Charge Modifying and pH Control Agents and Methods

To improve the transport characteristics of biologically active agents (including GRP, other active peptides and proteins, and macromolecular and small molecule drugs) for enhanced delivery across hydrophobic biological membrane barriers, the invention also provides techniques and reagents for charge modification of selected biologically active agents or delivery-enhancing agents described herein. In this regard, the relative permeabilities of macromolecules is generally be related to their partition coefficients. The degree of ionization of molecules, which is dependent on the pKₐ of the molecule and the pH at the biological membrane surface, also affects permeability of the molecules. Permeation and partitioning of biologically active agents, including GRP and analogs of the invention, for epithelial delivery may be facilitated by charge alteration or charge spreading of the active agent or permeabilizing agent, which is achieved, for example, by alteration of charged functional groups, by modifying the pH of the delivery vehicle or solution in which the active agent is delivered, or by coordinate administration of a charge- or pH-altering reagent with the active agent.

Consistent with these general teachings, epithelial delivery of charged macromolecular species, including GRP and other biologically active peptides and proteins, within the methods and compositions of the invention is substantially improved when the active agent is delivered to the epithelial surface in a substantially un-ionized, or neutral, electrical charge state.

Certain GRP and other biologically active peptide and protein components of epithelial formulations for use within the invention will be charge modified to yield an increase in the positive charge density of the peptide or protein. These modifications extend also to cationization of peptide and protein conjugates, carriers and other delivery forms disclosed herein. Cationization offers a convenient means of altering the biodistribution and transport properties of proteins and macromolecules within the invention. Cationization is undertaken in a manner that substantially preserves the biological activity of the active agent and limits potentially adverse side effects, including tissue damage and toxicity.

A “buffer” is generally used to maintain the pH of a solution at a nearly constant value. A buffer maintains the pH of a solution, even when small amounts of strong acid or strong base are added to the solution, by preventing or neutralizing large changes in concentrations of hydrogen and hydroxide ions. A buffer generally consists of a weak acid and its appropriate salt (or a weak base and its appropriate salt). The appropriate salt for a weak acid contains the same negative ion as present in the weak acid (see Lagowski, Macmillan Encyclopedia of Chemistry, Vol. 1, Simon & Schuster, New York, 1997, p. 273-4). The Henderson-Hasselbach Equation, pH=pKa+log 10[A−]/[HA], is used to describe a buffer, and is based on the standard equation for weak acid dissociation, HA≈H++A−. Examples of commonly used buffer sources include the following: glutamate, acetate, citrate, glycine, histidine, arginine, lysine, methionine, lactate, formate, glycolate, tartrate and mixtures thereof.

The “buffer capacity” means the amount of acid or base that can be added to a buffer solution before a significant pH change will occur. If the pH lies within the range of pK−1 and pK+1 of the weak acid the buffer capacity is appreciable, but outside this range it falls off to such an extent as to be of little value. Therefore, a given system only has a useful buffer action in a range of one pH unit on either side of the pK of the weak acid (or weak base) (see Dawson, Data for Biochemical Research, Third Edition, Oxford Science Publications, 1986, p. 419). Generally, suitable concentrations are chosen so that the pH of the solution is close to the pKa of the weak acid (or weak base) (see Lide, CRC Handbook of Chemistry and Physics, 86th Edition, Taylor & Francis Group, 2005-2006, p. 2-41). Further, solutions of strong acids and bases are not normally classified as buffer solutions, and they do not display buffer capacity between pH values 2.4 to 11.6.

### Degradative Enzyme Inhibitory Agents and Methods

Another excipient that may be included in a transepithelial preparation is a degradative enzyme inhibitor. Exemplary mucoadhesive polymer-enzyme inhibitor complexes that are useful within the epithelial delivery formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil-elastatinal (anti-elastase); Chitosan-antipain (anti-trypsin); Poly(acrylic acid)-bacitracin (anti-aminopeptidase N); Chitosan-EDTA (anti-aminopeptidase N, anti-carboxypeptidase A); Chitosan-EDTA-antipain (anti-trypsin, anti-chymotrypsin, anti-elastase). As described in further detail below, certain embodiments of the invention will optionally incorporate a novel chitosan derivative or chemically modified form of chitosan. One such novel derivative for use within the invention is denoted as a β-[1→4]-2-guanidino-2-deoxy-D-glucose polymer (poly-GuD).

Any inhibitor that inhibits the activity of an enzyme to protect the biologically active agent(s) may be usefully employed in the compositions and methods of the invention. Useful enzyme inhibitors for the protection of biologically active proteins and peptides include, for example, dipeptidyl aminopeptidase (DPP) IV inhibitors, soybean trypsin inhibitor, exendin trypsin inhibitor, chymotrypsin inhibitor and trypsin and chrymotrypsin inhibitor isolated from potato (solanum tuberosum L.) tubers. A combination or mixtures of inhibitors may be employed. Additional inhibitors of proteolytic enzymes for use within the invention include ovomucoid-enzyme, gabaxate mesylate, alpha1-antitrypsin, aprotinin, amastatin, bestatin, puromycin, bacitracin, leupepsin, alpha-macroglobulin, pepstatin and egg white or soybean trypsin inhibitor. These and other inhibitors can be used alone or in combination. The inhibitor(s) may be incorporated in or bound to a carrier, e.g., a hydrophilic polymer, coated on the surface of the dosage form which is to contact the nasal mucosa, or incorporated in the superficial phase of the surface, in combination with the biologically active agent or in a separately administered (e.g., pre-administered) formulation (e.g., oral pill).

The amount of the inhibitor, e.g., of a proteolytic enzyme inhibitor that is optionally incorporated in the compositions of the invention will vary depending on (a) the properties of the specific inhibitor, (b) the number of functional groups present in the molecule (which may be reacted to introduce ethylenic unsaturation necessary for copolymerization with hydrogel forming monomers), and (c) the number of lectin groups, such as glycosides, which are present in the inhibitor molecule. It may also depend on the specific therapeutic agent that is intended to be administered. Generally speaking, a useful amount of an enzyme inhibitor is from about 0.1 mg/ml to about 50 mg/ml, often from about 0.2 mg/ml to about 25 mg/ml, and more commonly from about 0.5 mg/ml to 5 mg/ml of the of the formulation (i.e., a separate protease inhibitor formulation or combined formulation with the inhibitor and biologically active agent).

In the case of trypsin inhibition, suitable inhibitors may be selected from, e.g., aprotinin, BBI, soybean trypsin inhibitor, chicken ovomucoid, chicken ovoinhibitor, human exendin trypsin inhibitor, camostat mesilate, flavonoid inhibitors, antipain, leupeptin, p-aminobenzamidine, AEBSF, TLCK (tosyllysine chloromethylketone), APMSF, DFP, PMSF, and poly(acrylate) derivatives. In the case of chymotrypsin inhibition, suitable inhibitors may be selected from, e.g., aprotinin, BBI, soybean trypsin inhibitor, chymostatin, benzyloxycarbonyl-Pro-Phe-CHO, FK-448, chicken ovoinhibitor, sugar biphenylboronic acids complexes, DFP, PMSF, β-phenylpropionate, and poly(acrylate) derivatives. In the case of elastase inhibition, suitable inhibitors may be selected from, e.g., elastatinal, methoxysuccinyl-Ala-Ala-Pro-Val-chloromethylketone (MPCMK), BBI, soybean trypsin inhibitor, chicken ovoinhibitor, DFP, and PMSF.

Additional enzyme inhibitors for use within the invention are selected from a wide range of non-protein inhibitors that vary in their degree of potency and toxicity. As described in further detail below, immobilization of these adjunct agents to matrices or other delivery vehicles, or development of chemically modified analogues, may be readily implemented to reduce or even eliminate toxic effects, when they are encountered. Among this broad group of candidate enzyme inhibitors for use within the invention are organophosphorous inhibitors, such as diisopropylfluorophosphate (DFP) and phenylmethylsulfonyl fluoride (PMSF), which are potent, irreversible inhibitors of serine proteases (e.g., trypsin and chymotrypsin). The additional inhibition of acetylcholinesterase by these compounds makes them highly toxic in uncontrolled delivery settings. Another candidate inhibitor, 4-(2-Aminoethyl)-benzenesulfonyl fluoride (AEBSF), has an inhibitory activity comparable to DFP and PMSF, but it is markedly less toxic. (4-Aminophenyl)-methanesulfonyl fluoride hydrochloride (APMSF) is another potent inhibitor of trypsin, but is toxic in uncontrolled settings. In contrast to these inhibitors, 4-(4-isopropylpiperadinocarbonyl)phenyl 1,2,3,4,-tetrahydro-1-naphthoate methanesulphonate (FK-448) is a low toxic substance, representing a potent and specific inhibitor of chymotrypsin. Further representatives of this non-protein group of inhibitor candidates, and also exhibiting low toxic risk, are camostat mesilate (N,N′-dimethyl carbamoylmethyl-p-(p′-guanidino-benzoyloxy)phenylacetate methane-sulphonate).

Yet another type of enzyme inhibitory agent for use within the methods and compositions of the invention are amino acids and modified amino acids that interfere with enzymatic degradation of specific therapeutic compounds. For use in this context, amino acids and modified amino acids are substantially non-toxic and can be produced at a low cost. However, due to their low molecular size and good solubility, they are readily diluted and absorbed in mucosal environments. Nevertheless, under proper conditions, amino acids can act as reversible, competitive inhibitors of protease enzymes. Certain modified amino acids can display a much stronger inhibitory activity. A desired modified amino acid in this context is known as a ‘transition-state’ inhibitor. The strong inhibitory activity of these compounds is based on their structural similarity to a substrate in its transition-state geometry, while they are generally selected to have a much higher affinity for the active site of an enzyme than the substrate itself. Transition-state inhibitors are reversible, competitive inhibitors. Examples of this type of inhibitor are α-aminoboronic acid derivatives, such as boro-leucine, boro-valine and boro-alanine. The boron atom in these derivatives can form a tetrahedral boronate ion that is believed to resemble the transition state of peptides during their hydrolysis by aminopeptidases. These amino acid derivatives are potent and reversible inhibitors of aminopeptidases and it is reported that boro-leucine is more than 100-times more effective in enzyme inhibition than bestatin and more than 1000-times more effective than puromycin. Another modified amino acid for which a strong protease inhibitory activity has been reported is N-acetylcysteine, which inhibits enzymatic activity of aminopeptidase N. This adjunct agent also displays mucolytic properties that can be employed within the methods and compositions of the invention to reduce the effects of the mucus diffusion barrier.

Still other useful enzyme inhibitors for use within the coordinate administration methods and combinatorial formulations of the invention may be selected from peptides and modified peptide enzyme inhibitors. An important representative of this class of inhibitors is the cyclic dodecapeptide, bacitracin, obtained from Bacillus licheniformis. In addition to these types of peptides, certain dipeptides and tripeptides display weak, non-specific inhibitory activity towards some protease. By analogy with amino acids, their inhibitory activity can be improved by chemical modifications. For example, phosphinic acid dipeptide analogues are also ‘transition-state’ inhibitors with a strong inhibitory activity towards aminopeptidases. They have reportedly been used to stabilize nasally administered leucine enkephalin. Another example of a transition-state analogue is the modified pentapeptide pepstatin, which is a very potent inhibitor of pepsin. Structural analysis of pepstatin, by testing the inhibitory activity of several synthetic analogues, demonstrated the major structure-function characteristics of the molecule responsible for the inhibitory activity. Another special type of modified peptide includes inhibitors with a terminally located aldehyde function in their structure. For example, the sequence benzyloxycarbonyl-Pro-Phe-CHO, which fulfills the known primary and secondary specificity requirements of chymotrypsin, has been found to be a potent reversible inhibitor of this target proteinase. The chemical structures of further inhibitors with a terminally located aldehyde function, e.g. antipain, leupeptin, chymostatin and elastatinal, are also known in the art, as are the structures of other known, reversible, modified peptide inhibitors, such as phosphoramidon, bestatin, puromycin and amastatin.

Due to their comparably high molecular mass, polypeptide protease inhibitors are more amenable than smaller compounds to concentrated delivery in a drug-carrier matrix. Additional agents for protease inhibition within the formulations and methods of the invention involve the use of complexing agents. These agents mediate enzyme inhibition by depriving the intranasal environment (or preparative or therapeutic composition) of divalent cations, which are co-factors for many proteases. For instance, the complexing agents EDTA and DTPA as coordinately administered or combinatorially formulated adjunct agents, in suitable concentration, will be sufficient to inhibit selected proteases to thereby enhance intranasal delivery of biologically active agents according to the invention. Further representatives of this class of inhibitory agents are EGTA, 1,10-phenanthroline and hydroxychinoline. In addition, due to their propensity to chelate divalent cations, these and other complexing agents are useful within the invention as direct, absorption-promoting agents.

As noted in more detail elsewhere herein, it is also contemplated to use various polymers, particularly mucoadhesive polymers, as enzyme inhibiting agents within the coordinate administration, multi-processing and/or combinatorial formulation methods and compositions of the invention. For example, poly(acrylate) derivatives, such as poly(acrylic acid) and polycarbophil, can affect the activity of various proteases, including trypsin, chymotrypsin. The inhibitory effect of these polymers may also be based on the complexation of divalent cations such as Ca²⁺ and Zn²⁺. It is further contemplated that these polymers may serve as conjugate partners or carriers for additional enzyme inhibitory agents, as described above. For example, a chitosan-EDTA conjugate has been developed and is useful within the invention that exhibits a strong inhibitory effect towards the enzymatic activity of zinc-dependent proteases. The mucoadhesive properties of polymers following covalent attachment of other enzyme inhibitors in this context are not expected to be substantially compromised, nor is the general utility of such polymers as a delivery vehicle for biologically active agents within the invention expected to be diminished. On the contrary, the reduced distance between the delivery vehicle and mucosal surface afforded by the mucoadhesive mechanism will minimize presystemic metabolism of the active agent, while the covalently bound enzyme inhibitors remain concentrated at the site of drug delivery, minimizing undesired dilution effects of inhibitors as well as toxic and other side effects caused thereby. In this manner, the effective amount of a coordinately administered enzyme inhibitor can be reduced due to the exclusion of dilution effects.

Exemplary mucoadhesive polymer-enzyme inhibitor complexes that are useful within the mucosal formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil-elastatinal (anti-elastase); Chitosan-antipain (anti-trypsin); Poly(acrylic acid)-bacitracin (anti-aminopeptidase N); Chitosan-EDTA (anti-aminopeptidase N, anti-carboxypeptidase A); Chitosan-EDTA-antipain (anti-trypsin, anti-chymotrypsin, anti-elastase).

### Mucolytic and Mucus-Clearing Agents and Methods

Effective delivery of biotherapeutic agents via intranasal administration must take into account the decreased drug transport rate across the protective mucus lining of the nasal mucosa, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. Mucus is secreted by randomly distributed secretory cells located in the nasal epithelium and in other mucosal epithelia. The structural unit of mucus is mucin. This glycoprotein is mainly responsible for the viscoelastic nature of mucus, although other macromolecules may also contribute to this property. In airway mucus, such macromolecules include locally produced secretory IgA, IgM, IgE, lysozyme, and bronchotransferrin, which also play an important role in host defense mechanisms.

The coordinate administration methods of the instant invention optionally incorporate effective mucolytic or mucus-clearing agents, which serve to degrade; thin or clear mucus from intranasal mucosal surfaces to facilitate absorption of intranasally administered biotherapeutic agents. Within these methods, a mucolytic or mucus-clearing agent is coordinately administered as an adjunct compound to enhance intranasal delivery of the biologically active agent. Alternatively, an effective amount of a mucolytic or mucus-clearing agent is incorporated as a processing agent within a multi-processing method of the invention, or as an additive within a combinatorial formulation of the invention, to provide an improved formulation that enhances intranasal delivery of biotherapeutic compounds by reducing the barrier effects of intranasal mucus.

A variety of mucolytic or mucus-clearing agents are available for incorporation within the methods and compositions of the invention. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order deoxycholate>taurocholate>glycocholate. Other effective agents that reduce mucus viscosity or adhesion to enhance intranasal delivery according to the methods of the invention include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of the invention include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%). These and other mucolytic or mucus-clearing agents are contacted with the nasal mucosa, typically in a concentration range of about 0.2 to 20 mM, coordinately with administration of the biologically active agent, to reduce the polar viscosity and/or elasticity of intranasal mucus.

Still other mucolytic or mucus-clearing agents may be selected from a range of glycosidase enzymes, which are able to cleave glycosidic bonds within the mucus glycoprotein. α-amylase and β-amylase are representative of this class of enzymes, although their mucolytic effect may be limited. In contrast, bacterial glycosidases which allow these microorganisms to permeate mucus layers of their hosts.

For combinatorial use with most biologically active agents within the invention, including peptide and protein therapeutics, non-ionogenic detergents are generally also useful as mucolytic or mucus-clearing agents. These agents typically will not modify or substantially impair the activity of therapeutic polypeptides.

### Viscosity Enhancing Agents

Viscosity enhancing or suspending agents may affect the rate of release of a drug from the dosage formulation and absorption. As a result viscosity enhancers can be used to modify permeation of some glucose-regulation peptides. Some examples of the materials which can serve as pharmaceutically acceptable viscosity enhancing agents are methylcellulose (MC); hydroxypropylmethylcellulose (HPMC); carboxymethylcellulose (CMC); cellulose; gelatin; starch; heta starch; poloxamers; pluronics; sodium CMC; sorbitol; acacia; povidone; carbopol; polycarbophil; chitosan; chitosan microspheres; alginate microspheres; chitosan glutamate; amberlite resin; hyaluronan; ethyl cellulose; maltodextrin DE; drum-dried way maize starch (DDWM); degradable starch microspheres (DSM); deoxyglycocholate (GDC); hydroxyethyl cellulose (HEC); hydroxypropyl cellulose (HPC); microcrystalline cellulose (MCC); polymethacrylic acid and polyethylene glycol; sulfobutylether B cyclodextrin; cross-linked eldexomer starch biospheres; sodiumtaurodihydrofusidate (STDHF); N-trimethyl chitosan chloride (TMC); degraded starch microspheres; amberlite resin; chistosan nanoparticles; spray-dried crospovidone; spray-dried dextran microspheres; spray-dried microcrystalline cellulose; and cross-linked eldexomer starch microspheres.

### Ciliostatic Agents and Methods

Because the self-cleaning capacity of certain mucosal tissues (e.g., nasal mucosal tissues) by mucociliary clearance is necessary as a protective function (e.g., to remove dust, allergens, and bacteria), it has been generally considered that this function should not be substantially impaired by mucosal medications. Mucociliary transport in the respiratory tract is a particularly important defense mechanism against infections. To achieve this function, ciliary beating in the nasal and airway passages moves a layer of mucus along the mucosa to removing inhaled particles and microorganisms.

Ciliostatic agents find use within the methods and compositions of the invention to increase the residence time of mucosally (e.g., intranasally) administered GRP, analogs and mimetics, and other biologically active agents disclosed herein. In particular, the delivery these agents within the methods and compositions of the invention is significantly enhanced in certain aspects by the coordinate administration or combinatorial formulation of one or more ciliostatic agents that function to reversibly inhibit ciliary activity of mucosal cells, to provide for a temporary, reversible increase in the residence time of the mucosally administered active agent(s). For use within these aspects of the invention, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance delivery of GRP, analogs and mimetics, and other biologically active agents disclosed herein, without unacceptable adverse side effects.

Various bacterial ciliostatic factors isolated and characterized in the literature may be employed within the embodiments of the invention. Ciliostatic factors from the bacterium Pseudomonas aeruginosa include a phenazine derivative, a pyo compound (2-alkyl-4-hydroxyquinolines), and a rhamnolipid (also known as a hemolysin). The pyo compound produced ciliostasis at concentrations of 50 μg/ml and without obvious ultrastructural lesions. The phenazine derivative also inhibited ciliary motility but caused some membrane disruption, although at substantially greater concentrations of 400 μg/ml. Limited exposure of tracheal explants to the rhamnolipid resulted in ciliostasis, which is associated with altered ciliary membranes. More extensive exposure to rhamnolipid is associated with removal of dynein arms from axonemes.

### Surface Active Agents and Methods

Within more detailed aspects of the invention, one or more membrane penetration-enhancing agents may be employed within a epithelial delivery method or formulation of the invention to enhance epithelial delivery of GRP, analogs and mimetics, and other biologically active agents disclosed herein. Biological membrane penetration enhancing agents in this context can be selected from: (i) a surfactant, (ii) a bile salt, (iii) a phospholipid additive, mixed micelle, liposome, or carrier, (iv) an alcohol, (v) an enamine, (vi) an NO donor compound, (vii) a long-chain amphipathic molecule (viii) a small hydrophobic penetration enhancer; (ix) sodium or a salicylic acid derivative; (x) a glycerol ester of acetoacetic acid (xi) a clyclodextrin or beta-cyclodextrin derivative, (xii) a medium-chain fatty acid, (xiii) a chelating agent, (xiv) an amino acid or salt thereof, (xv) an N-acetylamino acid or salt thereof, (xvi) an enzyme degradative to a selected membrane component, (xvii) an inhibitor of fatty acid synthesis, or (xviii) an inhibitor of cholesterol synthesis; or (xix) any combination of the membrane penetration enhancing agents recited in (i)-(xviii).

Certain surface-active agents (surfactants) are readily incorporated within the epithelial delivery formulations and methods of the invention as epithelial absorption enhancing agents. These agents, which may be coordinately administered or combinatorially formulated with GRP, analogs and mimetics, and other biologically active agents disclosed herein, may be selected from a broad assemblage of known surfactants. Surfactants, which generally fall into three classes: (1) nonionic polyoxyethylene ethers; (2) bile salts such as sodium glycocholate (SGC) and deoxycholate (DOC); and (3) derivatives of fusidic acid such as sodium taurodihydrofusidate (STDHF). The mechanisms of action of these various classes of surface-active agents typically include solubilization of the biologically active agent. For proteins and peptides which often form aggregates, the surface active properties of these absorption promoters can allow interactions with proteins such that smaller units such as surfactant coated monomers may be more readily maintained in solution. Examples of other surface-active agents are L-α-Phosphatidylcholine Didecanoyl (DDPC) polysorbate 80 and polysorbate 20. These monomers are presumably more transportable units than aggregates. A second potential mechanism is the protection of the peptide or protein from proteolytic degradation by proteases in the mucosal environment. Both bile salts and some fusidic acid derivatives reportedly inhibit proteolytic degradation of proteins by nasal homogenates at concentrations less than or equivalent to those required to enhance protein absorption. This protease inhibition may be especially important for peptides with short biological half-lives.

### Degradation Enzymes and Inhibitors of Fatty Acid and Cholesterol Synthesis

In related aspects of the invention, GRP, analogs and mimetics, and other biologically active agents for biological membrane administration are formulated or coordinately administered with a penetration enhancing agent selected from a degradation enzyme, or a metabolic stimulatory agent or inhibitor of synthesis of fatty acids, sterols or other selected epithelial barrier components, U.S. Pat. No. 6,190,894. For example, degradative enzymes such as phospholipase, hyaluronidase, neuraminidase, and chondroitinase may be employed to enhance mucosal penetration of GRP, analogs and mimetics, and other biologically active agent without causing irreversible damage to the mucosal barrier. In one embodiment, chondroitinase is employed within a method or composition as provided herein to alter glycoprotein or glycolipid constituents of the permeability barrier of the mucosa, thereby enhancing mucosal absorption of GRP, analogs and mimetics, and other biologically active agents disclosed herein.

With regard to inhibitors of synthesis of mucosal barrier constituents, it is noted that free fatty acids account for 20-25% of epithelial lipids by weight. Two rate-limiting enzymes in the biosynthesis of free fatty acids are acetyl CoA carboxylase and fatty acid synthetase. Through a series of steps, free fatty acids are metabolized into phospholipids. Thus, inhibitors of free fatty acid synthesis and metabolism for use within the methods and compositions of the invention include, but are not limited to, inhibitors of acetyl CoA carboxylase such as S-tetradecyloxy-2-furancarboxylic acid (TOFA); inhibitors of fatty acid synthetase; inhibitors of phospholipase A such as gomisin A, 2-(p-amylcinnamyl)amino-4-chlorobenzoic acid, bromophenacyl bromide, monoalide, 7,7-dimethyl-5,8-eicosadienoic acid, nicergoline, cepharanthine, nicardipine, quercetin, dibutyryl-cyclic AMP, R-24571, N-oleoylethanolamine, N-(7-nitro-2,1,3-benzoxadiazol-4-yl) phosphostidyl serine, cyclosporine A, topical anesthetics, including dibucaine, prenylamine, retinoids, such as all-trans and 13-cis-retinoic acid, W-7, trifluoperazine, R-24571 (calmidazolium), 1-hexadocyl-3-trifluoroethyl glycero-sn-2-phosphomenthol (MJ33); calcium channel blockers including nicardipine, verapamil, diltiazem, nifedipine, and nimodipine; antimalarials including quinacrine, mepacrine, chloroquine and hydroxychloroquine; beta blockers including propanalol and labetalol; calmodulin antagonists; EGTA; thimersol; glucocorticosteroids including dexamethasone and prednisolone; and nonsteroidal antiinflammatory agents including indomethacin and naproxen.

Free sterols, primarily cholesterol, account for 20-25% of the epithelial lipids by weight. The rate limiting enzyme in the biosynthesis of cholesterol is 3-hydroxy-3-methylglutaryl (HMG) CoA reductase. Inhibitors of cholesterol synthesis for use within the methods and compositions of the invention include, but are not limited to, competitive inhibitors of (HMG) CoA reductase, such as simvastatin, lovastatin, fluindostatin (fluvastatin), pravastatin, mevastatin, as well as other HMG CoA reductase inhibitors, such as cholesterol oleate, cholesterol sulfate and phosphate, and oxygenated sterols, such as 25-OH— and 26-OH— cholesterol; inhibitors of squalene synthetase; inhibitors of squalene epoxidase; inhibitors of DELTA7 or DELTA24 reductases such as 22,25-diazacholesterol, 20,25-diazacholestenol, AY9944, and triparanol.

Each of the inhibitors of fatty acid synthesis or the sterol synthesis inhibitors may be coordinately administered or combinatorially formulated with one or more GRP, analogs and mimetics, and other biologically active agents disclosed herein to achieve enhanced epithelial penetration of the active agent(s). An effective concentration range for the sterol inhibitor in a therapeutic or adjunct formulation for mucosal delivery is generally from about 0.0001% to about 20% by weight of the total, more typically from about 0.01% to about 5%.

### Nitric Oxide Donor Agents and Methods

Within other related aspects of the invention, a nitric oxide (NO) donor is selected as a biological membrane penetration-enhancing agent to enhance epithelial delivery of one or more GRP, analogs and mimetics, and other biologically active agents disclosed herein. Various NO donors are known in the art and are useful in effective concentrations within the methods and formulations of the invention. Exemplary NO donors include, but are not limited to, nitroglycerine, nitropruside, NOC5 [3-(2-hydroxy-1-(methyl-ethyl)-2-nitrosohydrazino)-1-propanamine], NOC12 [N-ethyl-2-(1-ethyl-hydroxy-2-nitrosohydrazino)-ethanamine], SNAP [S-nitroso-N-acetyl-DL-penicillamine], NORI and NOR4. Within the methods and compositions of the invention, an effective amount of a selected NO donor is coordinately administered or combinatorially formulated with one or more GRP, analogs and mimetics, and/or other biologically active agents disclosed herein, into or through the epithelium.

Agents for Modulating Epithelial Junction Structure and/or Physiology

The present invention provides pharmaceutical composition that contains one or more GRP, analogs or mimetics, and/or other biologically active agents in combination with epithelial delivery enhancing agents disclosed herein formulated in a pharmaceutical preparation for epithelial delivery.

The permeabilizing agent reversibly enhances mucosal epithelial paracellular transport, typically by modulating epithelial junctional structure and/or physiology at a mucosal epithelial surface in the subject. This effect typically involves inhibition by the permeabilizing agent of homotypic or heterotypic binding between epithelial membrane adhesive proteins of neighboring epithelial cells. Target proteins for this blockade of homotypic or heterotypic binding can be selected from various related junctional adhesion molecules (JAMs), occludins, or claudins. Examples of this are antibodies, antibody fragments or single-chain antibodies that bind to the extracellular domains of these proteins.

In yet additional detailed embodiments, the invention provides permeabilizing peptides and peptide analogs and mimetics for enhancing mucosal epithelial paracellular transport. The subject peptides and peptide analogs and mimetics typically work within the compositions and methods of the invention by modulating epithelial junctional structure and/or physiology in a mammalian subject. In certain embodiments, the peptides and peptide analogs and mimetics effectively inhibit homotypic and/or heterotypic binding of an epithelial membrane adhesive protein selected from a junctional adhesion molecule (JAM), occludin, or claudin.

One such agent that has been extensively studied is the bacterial toxin from Vibrio cholerae known as the “zonula occludens toxin” (ZOT). This toxin mediates increased intestinal mucosal permeability and causes disease symptoms including diarrhea in infected subjects. Fasano, et al, Proc. Nat. Acad. Sci., U.S.A. 8:5242-5246, 1991. When tested on rabbit ileal mucosa, ZOT increased the intestinal permeability by modulating the structure of intercellular tight junctions. More recently, it has been found that ZOT is capable of reversibly opening tight junctions in the intestinal mucosa. It has also been reported that ZOT is capable of reversibly opening tight junctions in the nasal mucosa. U.S. Pat. No. 5,908,825.

Within the methods and compositions of the invention, ZOT, as well as various analogs and mimetics of ZOT that function as agonists or antagonists of ZOT activity, are useful for enhancing intranasal delivery of biologically active agents—by increasing paracellular absorption into and across the nasal mucosa. In this context, ZOT typically acts by causing a structural reorganization of tight junctions marked by altered localization of the junctional protein ZO1. Within these aspects of the invention, ZOT is coordinately administered or combinatorially formulated with the biologically active agent in an effective amount to yield significantly enhanced absorption of the active agent, by reversibly increasing nasal mucosal permeability without substantial adverse side effects.

### Vasodilator Agents and Methods

Yet another class of absorption-promoting agents that shows beneficial utility within the coordinate administration and combinatorial formulation methods and compositions of the invention are vasoactive compounds, more specifically vasodilators. These compounds function within the invention to modulate the structure and physiology of the submucosal vasculature, increasing the transport rate of GRP, analogs and mimetics, and other biologically active agents into or through the epithelium and/or to specific target tissues or compartments (e.g., the systemic circulation or central nervous system).

Vasodilator agents for use within the invention typically cause submucosal blood vessel relaxation by either a decrease in cytoplasmic calcium, an increase in nitric oxide (NO) or by inhibiting myosin light chain kinase. They are generally divided into 9 classes: calcium antagonists, potassium channel openers, ACE inhibitors, angiotensin-II receptor antagonists, α-adrenergic and imidazole receptor antagonists, β1-adrenergic agonists, phosphodiesterase inhibitors, eicosanoids and NO donors.

Despite chemical differences, the pharmacokinetic properties of calcium antagonists are similar. Absorption into the systemic circulation is high, and these agents therefore undergo considerable first-pass metabolism by the liver, resulting in individual variation in pharmacokinetics. Except for the newer drugs of the dihydropyridine type (amlodipine, felodipine, isradipine, nilvadipine, nisoldipine and nitrendipine), the half-life of calcium antagonists is short. Therefore, to maintain an effective drug concentration for many of these may require delivery by multiple dosing, or controlled release formulations, as described elsewhere herein. Treatment with the potassium channel opener minoxidil may also be limited in manner and level of administration due to potential adverse side effects.

ACE inhibitors prevent conversion of angiotensin-I to angiotensin-II, and are most effective when renin production is increased. Since ACE is identical to kininase-II, which inactivates the potent endogenous vasodilator bradykinin, ACE inhibition causes a reduction in bradykinin degradation. ACE inhibitors provide the added advantage of cardioprotective and cardioreparative effects, by preventing and reversing cardiac fibrosis and ventricular hypertrophy in animal models. The predominant elimination pathway of most ACE inhibitors is via renal excretion. Therefore, renal impairment is associated with reduced elimination and a dosage reduction of 25 to 50% is recommended in patients with moderate to severe renal impairment.

With regard to NO donors, these compounds are particularly useful within the invention for their additional effects on mucosal permeability. In addition to the above-noted NO donors, complexes of NO with nucleophiles called NO/nucleophiles, or NONOates, spontaneously and nonenzymatically release NO when dissolved in aqueous solution at physiologic pH. In contrast, nitro vasodilators such as nitroglycerin require specific enzyme activity for NO release. NONOates release NO with a defined stoichiometry and at predictable rates ranging from <3 minutes for diethylamine/NO to approximately 20 hours for diethylenetriamine/NO (DETANO).

Within certain methods and compositions of the invention, a selected vasodilator agent is coordinately administered (e.g., systemically or intranasally, simultaneously or in combinatorially effective temporal association) or combinatorially formulated with one or more GRP, analogs and mimetics, and other biologically active agent(s) in an amount effective to enhance the mucosal absorption of the active agent(s) to reach a target tissue or compartment in the subject (e.g., the liver, hepatic portal vein, CNS tissue or fluid, or blood plasma).

### Selective Transport-Enhancing Agents and Methods

The compositions and delivery methods of the invention optionally incorporate a selective transport-enhancing agent that facilitates transport of one or more biologically active agents. These transport-enhancing agents may be employed in a combinatorial formulation or coordinate administration protocol with one or more of the GRP, analogs and mimetics disclosed herein, to coordinately enhance delivery of one or more additional biologically active agent(s) across biological membrane transport barriers, to enhance epithelial delivery of the active agent(s) to reach a target tissue or compartment in the subject (e.g., the mucosal epithelium, liver, CNS tissue or fluid, or blood plasma). Alternatively, the transport-enhancing agents may be employed in a combinatorial formulation or coordinate administration protocol to directly enhance epithelial delivery of one or more of the GRP, analogs and mimetics, with or without enhanced delivery of an additional biologically active agent.

Exemplary selective transport-enhancing agents for use within this aspect of the invention include, but are not limited to, glycosides, sugar-containing molecules, and binding agents such as lectin binding agents, which are known to interact specifically with epithelial transport barrier components. For example, specific “bioadhesive” ligands, including various plant and bacterial lectins, which bind to cell surface sugar moieties by receptor-mediated interactions can be employed as carriers or conjugated transport mediators for enhancing mucosal, e.g., nasal delivery of biologically active agents within the invention. Certain bioadhesive ligands for use within the invention will mediate transmission of biological signals to epithelial target cells that trigger selective uptake of the adhesive ligand by specialized cellular transport processes (endocytosis or transcytosis). These transport mediators can therefore be employed as a “carrier system” to stimulate or direct selective uptake of one or more GRP, analogs and mimetics, and other biologically active agent(s) into and/or through mucosal epithelia. These and other selective transport-enhancing agents significantly enhance epithelial delivery of macromolecular biopharmaceuticals (particularly peptides, proteins, oligonucleotides and polynucleotide vectors) within the invention. Lectins are plant proteins that bind to specific sugars found on the surface of glycoproteins and glycolipids of eukaryotic cells. Concentrated solutions of lectins have a ‘mucotractive’ effect, and various studies have demonstrated rapid receptor mediated endocytocis (RME) of lectins and lectin conjugates (e.g., concanavalin A conjugated with colloidal gold particles) across mucosal surfaces. Additional studies have reported that the uptake mechanisms for lectins can be utilized for intestinal drug targeting in vivo. In certain of these studies, polystyrene nanoparticles (500 nm) were covalently coupled to tomato lectin and reported yielded improved systemic uptake after oral administration to rats.

In addition to plant lectins, microbial adhesion and invasion factors provide a rich source of candidates for use as adhesive/selective transport carriers within the mucosal delivery methods and compositions of the invention. Two components are necessary for bacterial adherence processes, a bacterial ‘adhesin’ (adherence or colonization factor) and a receptor on the host cell surface. Bacteria causing mucosal infections need to penetrate the mucus layer before attaching themselves to the epithelial surface. This attachment is usually mediated by bacterial fimbriae or pilus structures, although other cell surface components may also take part in the process. Adherent bacteria colonize mucosal epithelia by multiplication and initiation of a series of biochemical reactions inside the target cell through signal transduction mechanisms (with or without the help of toxins). Associated with these invasive mechanisms, a wide diversity of bioadhesive proteins (e.g., invasin, internalin) originally produced by various bacteria and viruses are known. These allow for extracellular attachment of such microorganisms with an impressive selectivity for host species and even particular target tissues. Signals transmitted by such receptor-ligand interactions trigger the transport of intact, living microorganisms into, and eventually through, epithelial cells by endo- and transcytotic processes. Such naturally occurring phenomena may be harnessed (e.g., by complexing biologically active agents such as GRP with an adhesin) according to the teachings herein for enhanced delivery of biologically active compounds into or across a biological membrane and/or to other designated target sites of drug action.

Various bacterial and plant toxins that bind epithelial surfaces in a specific, lectin-like manner are also useful within the methods and compositions of the invention. For example, diptheria toxin (DT) enters host cells rapidly by RME. Likewise, the B subunit of the E. coli heat labile toxin binds to the brush border of intestinal epithelial cells in a highly specific, lectin-like manner. Uptake of this toxin and transcytosis to the basolateral side of the enterocytes has been reported in vivo and in vitro. Other researches have expressed the transmembrane domain of diphtheria toxin in E. coli as a maltose-binding fusion protein and coupled it chemically to high-Mw poly-L-lysine. The resulting complex is successfully used to mediate internalization of a reporter gene in vitro. In addition to these examples, Staphylococcus aureus produces a set of proteins (e.g., staphylococcal enterotoxin A (SEA), SEB, toxic shock syndrome toxin 1 (TSST-1) which act both as superantigens and toxins. Studies relating to these proteins have reported dose-dependent, facilitated transcytosis of SEB and TSST-1 in Caco-2 cells.

Viral haemagglutinins comprise another type of transport agent to facilitate mucosal delivery of biologically active agents within the methods and compositions of the invention. The initial step in many viral infections is the binding of surface proteins (haemagglutinins) to mucosal cells. These binding proteins have been identified for most viruses, including rotaviruses, varicella zoster virus, semliki forest virus, adenoviruses, potato leafroll virus, and reovirus. These and other exemplary viral hemagglutinins can be employed in a combinatorial formulation (e.g., a mixture or conjugate formulation) or coordinate administration protocol with one or more of the GRP, analogs and mimetics disclosed herein, to coordinately enhance mucosal delivery of one or more additional biologically active agent(s). Alternatively, viral hemagglutinins can be employed in a combinatorial formulation or coordinate administration protocol to directly enhance mucosal delivery of one or more of the GRP, analogs and mimetics, with or without enhanced delivery of an additional biologically active agent.

A variety of endogenous, selective transport-mediating factors are also available for use within the invention. Mammalian cells have developed an assortment of mechanisms to facilitate the internalization of specific substrates and target these to defined compartments. Collectively, these processes of membrane deformations are termed ‘endocytosis’ and comprise phagocytosis, pinocytosis, receptor-mediated endocytosis (clathrin-mediated RME), and potocytosis (non-clathrin-mediated RME). RME is a highly specific cellular biologic process by which, as its name implies, various ligands bind to cell surface receptors and are subsequently internalized and trafficked within the cell. In many cells the process of endocytosis is so active that the entire membrane surface is internalized and replaced in less than a half hour. Two classes of receptors are proposed based on their orientation in the cell membrane; the amino terminus of Type I receptors is located on the extracellular side of the membrane, whereas Type II receptors have this same protein tail in the intracellular milieu.

Still other embodiments of the invention utilize transferrin as a carrier or stimulant of RME of epithelially delivered biologically active agents. Transferrin, an 80 kDa iron-transporting glycoprotein, is efficiently taken up into cells by RME. Transferrin receptors are found on the surface of most proliferating cells, in elevated numbers on erythroblasts and on many kinds of tumors. The transcytosis of transferrin (Tf) and transferrin conjugates is reportedly enhanced in the presence of Brefeldin A (BFA), a fungal metabolite. In other studies, BFA treatment has been reported to rapidly increase apical endocytosis of both ricin and HRP in MDCK cells. Thus, BFA and other agents that stimulate receptor-mediated transport can be employed within the methods of the invention as combinatorially formulated (e.g., conjugated) and/or coordinately administered agents to enhance receptor-mediated transport of biologically active agents, including GRP, analogs and mimetics.

### Polymeric Delivery Vehicles and Methods

Within certain aspects of the invention, GRP, analogs and mimetics, other biologically active agents disclosed herein, and delivery-enhancing agents as described above, are, individually or combinatorially, incorporated within a epithelially (e.g., nasally) administered formulation that includes a biocompatible polymer functioning as a carrier or base. Such polymer carriers include polymeric powders, matrices or microparticulate delivery vehicles, among other polymer forms. The polymer can be of plant, animal, or synthetic origin. Often the polymer is crosslinked. Additionally, in these delivery systems the GRP, analog or mimetic, can be functionalized in a manner where it can be covalently bound to the polymer and rendered inseparable from the polymer by simple ishing. In other embodiments, the polymer is chemically modified with an inhibitor of enzymes or other agents which may degrade or inactivate the biologically active agent(s) and/or delivery enhancing agent(s). In certain formulations, the polymer is a partially or completely water insoluble but water swellable polymer, e.g., a hydrogel. Polymers useful in this aspect of the invention are desirably water interactive and/or hydrophilic in nature to absorb significant quantities of water, and they often form hydrogels when placed in contact with water or aqueous media for a period of time sufficient to reach equilibrium with water. In more detailed embodiments, the polymer is a hydrogel which, when placed in contact with excess water, absorbs at least two times its weight of water at equilibrium when exposed to water at room temperature, U.S. Pat. No. 6,004,583.

Drug delivery systems based on biodegradable polymers are preferred in many biomedical applications because such systems are broken down either by hydrolysis or by enzymatic reaction into non-toxic molecules. The rate of degradation is controlled by manipulating the composition of the biodegradable polymer matrix. These types of systems can therefore be employed in certain settings for long-term release of biologically active agents. Biodegradable polymers such as poly(glycolic acid) (PGA), poly-(lactic acid) (PLA), and poly(D,L-lactic-co-glycolic acid) (PLGA), have received considerable attention as possible drug delivery carriers, since the degradation products of these polymers have been found to have low toxicity. During the normal metabolic function of the body these polymers degrade into carbon dioxide and water. These polymers have also exhibited excellent biocompatibility.

For prolonging the biological activity of GRP, analogs and mimetics, and other biologically active agents disclosed herein, as well as optional delivery-enhancing agents, these agents may be incorporated into polymeric matrices, e.g., polyorthoesters, polyanhydrides, or polyesters. This yields sustained activity and release of the active agent(s), e.g., as determined by the degradation of the polymer matrix. Although the encapsulation of biotherapeutic molecules inside synthetic polymers may stabilize them during storage and delivery, the largest obstacle of polymer-based release technology is the activity loss of the therapeutic molecules during the formulation processes that often involve heat, sonication or organic solvents.

Absorption-promoting polymers contemplated for use within the invention may include derivatives and chemically or physically modified versions of the foregoing types of polymers, in addition to other naturally occurring or synthetic polymers, gums, resins, and other agents, as well as blends of these materials with each other or other polymers, so long as the alterations, modifications or blending do not adversely affect the desired properties, such as water absorption, hydrogel formation, and/or chemical stability for useful application. In more detailed aspects of the invention, polymers such as nylon, acrylan and other normally hydrophobic synthetic polymers may be sufficiently modified by reaction to become water swellable and/or form stable gels in aqueous media.

Absorption-promoting polymers of the invention may include polymers from the group of homo- and copolymers based on various combinations of the following vinyl monomers: acrylic and methacrylic acids, acrylamide, methacrylamide, hydroxyethylacrylate or methacrylate, vinylpyrrolidones, as well as polyvinylalcohol and its co- and terpolymers, polyvinylacetate, its co- and terpolymers with the above listed monomers and 2-acrylamido-2-methyl-propanesulfonic acid (AMPS®). Very useful are copolymers of the above listed monomers with copolymerizable functional monomers such as acryl or methacryl amide acrylate or methacrylate esters where the ester groups are derived from straight or branched chain alkyl, aryl having up to four aromatic rings which may contain alkyl substituents of 1 to 6 carbons; steroidal, sulfates, phosphates or cationic monomers such as N,N-dimethylaminoalkyl(meth)acrylamide, dimethylaminoalkyl(meth)acrylate, (meth)acryloxyalkyltrimethylammonium chloride, (meth)acryloxyalkyldimethylbenzyl ammonium chloride.

Additional absorption-promoting polymers for use within the invention are those classified as dextrans, dextrins, and from the class of materials classified as natural gums and resins, or from the class of natural polymers such as processed collagen, chitin, chitosan, pullalan, zooglan, alginates and modified alginates such as “Kelcoloid” (a polypropylene glycol modified alginate) gellan gums such as “Kelocogel”, Xanathan gums such as “Keltrol”, estastin, alpha hydroxy butyrate and its copolymers, hyaluronic acid and its derivatives, polylactic and glycolic acids.

A very useful class of polymers applicable within the instant invention are olefinically-unsaturated carboxylic acids containing at least one activated carbon-to-carbon olefinic double bond, and at least one carboxyl group; that is, an acid or functional group readily converted to an acid containing an olefinic double bond which readily functions in polymerization because of its presence in the monomer molecule, either in the alpha-beta position with respect to a carboxyl group, or as part of a terminal methylene grouping. Olefinically-unsaturated acids of this class include such materials as the acrylic acids typified by the acrylic acid itself, alpha-cyano acrylic acid, beta methylacrylic acid (crotonic acid), alpha-phenyl acrylic acid, beta-acryloxy propionic acid, cinnamic acid, p-chloro cinnamic acid, 1-carboxy-4-phenyl butadiene-1,3, itaconic acid, citraconic acid, mesaconic acid, glutaconic acid, aconitic acid, maleic acid, furmaric acid, and tricarboxy ethylene. As used herein, the term “carboxylic acid” includes the polycarboxylic acids and those acid anhydrides, such as maleic anhydride, wherein the anhydride group is formed by the elimination of one molecule of water from two carboxyl groups located on the same carboxylic acid molecule.

Representative acrylates useful as absorption-promoting agents within the invention include methyl acrylate, ethyl acrylate, propyl acrylate, isopropyl acrylate, butyl acrylate, isobutyl acrylate, methyl methacrylate, methyl ethacrylate, ethyl methacrylate, octyl acrylate, heptyl acrylate, octyl methacrylate, isopropyl methacrylate, 2-ethylhexyl methacrylate, nonyl acrylate, hexyl acrylate, n-hexyl methacrylate, and the like. Higher alkyl acrylic esters are decyl acrylate, isodecyl methacrylate, lauryl acrylate, stearyl acrylate, behenyl acrylate and melissyl acrylate and methacrylate versions thereof. Mixtures of two or three or more long chain acrylic esters may be successfully polymerized with one of the carboxylic monomers. Other comonomers include olefins, including alpha olefins, vinyl ethers, vinyl esters, and mixtures thereof.

Other vinylidene monomers, including the acrylic nitriles, may also be used as absorption-promoting agents within the methods and compositions of the invention to enhance delivery and absorption of one or more GRP, analogs and mimetics, and other biologically active agent(s), including to enhance delivery of the active agent(s) to a target tissue or compartment in the subject (e.g., the liver, hepatic portal vein, CNS tissue or fluid, or blood plasma). Useful alpha, beta-olefinically unsaturated nitriles are preferably monoolefinically unsaturated nitriles having from 3 to 10 carbon atoms such as acrylonitrile, methacrylonitrile, and the like. Most preferred are acrylonitrile and methacrylonitrile. Acrylic amides containing from 3 to 35 carbon atoms including monoolefinically unsaturated amides also may be used. Representative amides include acrylamide, methacrylamide, N-t-butyl acrylamide, N-cyclohexyl acrylamide, higher alkyl amides, where the alkyl group on the nitrogen contains from 8 to 32 carbon atoms, acrylic amides including N-alkylol amides of alpha, beta-olefinically unsaturated carboxylic acids including those having from 4 to 10 carbon atoms such as N-methylol acrylamide, N-propanol acrylamide, N-methylol methacrylamide, N-methylol maleimide, N-methylol maleamic acid esters, N-methylol-p-vinyl benzamide, and the like.

Yet additional useful absorption promoting materials are alpha-olefins containing from 2 to 18 carbon atoms, more preferably from 2 to 8 carbon atoms; dienes containing from 4 to 10 carbon atoms; vinyl esters and allyl esters such as vinyl acetate; vinyl aromatics such as styrene, methyl styrene and chloro-styrene; vinyl and allyl ethers and ketones such as vinyl methyl ether and methyl vinyl ketone; chloroacrylates; cyanoalkyl acrylates such as alpha-cyanomethyl acrylate, and the alpha-, beta-, and gamma-cyanopropyl acrylates; alkoxyacrylates such as methoxy ethyl acrylate; haloacrylates as chloroethyl acrylate; vinyl halides and vinyl chloride, vinylidene chloride and the like; divinyls, diacrylates and other polyfunctional monomers such as divinyl ether, diethylene glycol diacrylate, ethylene glycol dimethacrylate, methylene-bis-acrylamide, allylpentaerythritol, and the like; and bis(beta-haloalkyl) alkenyl phosphonates such as bis(beta-chloroethyl) vinyl phosphonate and the like as are known to those skilled in the art. Copolymers wherein the carboxy containing monomer is a minor constituent, and the other vinylidene monomers present as major components are readily prepared in accordance with the methods disclosed herein.

When hydrogels are employed as absorption promoting agents within the invention, these may be composed of synthetic copolymers from the group of acrylic and methacrylic acids, acrylamide, methacrylamide, hydroxyethylacrylate (HEA) or methacrylate (HEMA), and vinylpyrrolidones which are water interactive and swellable. Specific illustrative examples of useful polymers, especially for the delivery of peptides or proteins, are the following types of polymers: (meth)acrylamide and 0.1 to 99 wt. % (meth)acrylic acid; (meth)acrylamides and 0.1-75 wt % (meth)acryloxyethyl trimethyammonium chloride; (meth)acrylamide and 0.1-75 wt % (meth)acrylamide; acrylic acid and 0.1-75 wt % alkyl(meth)acrylates; (meth)acrylamide and 0.1-75 wt % AMPS® (trademark of Lubrizol Corp.); (meth)acrylamide and 0 to 30 wt % alkyl(meth)acrylamides and 0.1-75 wt % AMPS®; (meth)acrylamide and 0.1-99 wt. % HEMA; (metb)acrylamide and 0.1 to 75 wt % HEMA and 0.1 to 99% (meth)acrylic acid; (meth)acrylic acid and 0.1-99 wt % HEMA; 50 mole % vinyl ether and 50 mole % maleic anhydride; (meth)acrylamide and 0.1 to 75 wt % (meth)acryloxyalkyl dimethyl benzylammonium chloride; (meth)acrylamide and 0.1 to 99 wt % vinyl pyrrolidone; (meth)acrylamide and 50 wt % vinyl pyrrolidone and 0.1-99.9 wt % (meth)acrylic acid; (meth)acrylic acid and 0.1 to 75 wt % AMPS® and 0.1-75 wt % alkyl(meth)acrylamide. In the above examples, alkyl means C₁ to C₃₀, preferably C₁ to C₂₂, linear and branched and C₄ to C₁₆ cyclic; where (meth) is used, it means that the monomers with and without the methyl group are included. Other very useful hydrogel polymers are swellable, but insoluble versions of poly(vinyl pyrrolidone) starch, carboxymethyl cellulose and polyvinyl alcohol.

Additional polymeric hydrogel materials useful within the invention include (poly) hydroxyalkyl(meth)acrylate: anionic and cationic hydrogels: poly(electrolyte) complexes; poly(vinyl alcohols) having a low acetate residual: a swellable mixture of crosslinked agar and crosslinked carboxymethyl cellulose: a swellable composition comprising methyl cellulose mixed with a sparingly crosslinked agar; a water swellable copolymer produced by a dispersion of finely divided copolymer of maleic anhydride with styrene, ethylene, propylene, or isobutylene; a water swellable polymer of N-vinyl lactams; swellable sodium salts of carboxymethyl cellulose; and the like.

Other gelable, fluid imbibing and retaining polymers useful for forming the hydrophilic hydrogel for mucosal delivery of biologically active agents within the invention include pectin; polysaccharides such as agar, acacia, karaya, tragacenth, algins and guar and their cross linked versions; acrylic acid polymers, copolymers and salt derivatives, polyacrylamides; water swellable indene maleic anhydride polymers; starch graft copolymers; acrylate type polymers and copolymers with water absorbability of about 2 to 400 times its original weight; diesters of polyglucan; a mixture of cross linked poly(vinyl alcohol) and poly(N-vinyl-2-pyrrolidone); polyoxybutylene-polyethylene block copolymer gels; carob gum; polyester gels; poly urea gels; polyether gels; polyamide gels; polyimide gels; polypeptide gels; polyamino acid gels; poly cellulosic gels; crosslinked indene-maleic anhydride acrylate polymers; and polysaccharides.

Synthetic hydrogel polymers for use within the invention may be made by an infinite combination of several monomers in several ratios. The hydrogel can be crosslinked and generally possesses the ability to imbibe and absorb fluid and swell or expand to an enlarged equilibrium state. The hydrogel typically swells or expands upon delivery to the nasal mucosal surface, absorbing about 2-5, 5-10, 10-50, up to 50-100 or more times fold its weight of water. The optimum degree of swellability for a given hydrogel will be determined for different biologically active agents depending upon such factors as molecular weight, size, solubility and diffusion characteristics of the active agent carried by or entrapped or encapsulated within the polymer, and the specific spacing and cooperative chain motion associated with each individual polymer.

Hydrophilic polymers useful within the invention are water insoluble but water swellable. Such water-swollen polymers as typically referred to as hydrogels or gels. Such gels may be conveniently produced from water-soluble polymer by the process of crosslinking the polymers by a suitable crosslinking agent. However, stable hydrogels may also be formed from specific polymers under defined conditions of pH, temperature and/or ionic concentration, according to know methods in the art. Typically the polymers are cross-linked, that is, cross-linked to the extent that the polymers possess good hydrophilic properties, have improved physical integrity (as compared to non cross-linked polymers of the same or similar type) and exhibit improved ability to retain within the gel network both the biologically active agent of interest and additional compounds for coadministration therewith such as a cytokine or enzyme inhibitor, while retaining the ability to release the active agent(s) at the appropriate location and time.

Generally hydrogel polymers for use within the invention are crosslinked with a difunctional cross-linking in the amount of from 0.01 to 25 weight percent, based on the weight of the monomers forming the copolymer, and more preferably from 0.1 to 20 weight percent and more often from 0.1 to 15 weight percent of the crosslinking agent. Another useful amount of a crosslinking agent is 0.1 to 10 weight percent. Tri, tetra or higher multifunctional crosslinking agents may also be employed. When such reagents are utilized, lower amounts may be required to attain equivalent crosslinking density, i.e., the degree of crosslinking, or network properties that are sufficient to contain effectively the biologically active agent(s).

The crosslinks can be covalent, ionic or hydrogen bonds with the polymer possessing the ability to swell in the presence of water containing fluids. Such crosslinkers and crosslinking reactions are known to those skilled in the art and in many cases are dependent upon the polymer system. Thus a crosslinked network may be formed by free radical copolymerization of unsaturated monomers. Polymeric hydrogels may also be formed by crosslinking preformed polymers by reacting functional groups found on the polymers such as alcohols, acids, amines with such groups as glyoxal, formaldehyde or glutaraldehyde, bis anhydrides and the like.

The polymers also may be cross-linked with any polyene, e.g. decadiene or trivinyl cyclohexane; acrylamides, such as N,N-methylene-bis(acrylamide); polyfunctional acrylates, such as trimethylol propane triacrylate; or polyfunctional vinylidene monomer containing at least 2 terminal CH₂<groups, including, for example, divinyl benzene, divinyl naphthlene, allyl acrylates and the like. In certain embodiments, cross-linking monomers for use in preparing the copolymers are polyalkenyl polyethers having more than one alkenyl ether grouping per molecule, which may optionally possess alkenyl groups in which an olefinic double bond is present attached to a terminal methylene grouping (e.g., made by the etherification of a polyhydric alcohol containing at least 2 carbon atoms and at least 2 hydroxyl groups). Compounds of this class may be produced by reacting an alkenyl halide, such as allyl chloride or allyl bromide, with a strongly alkaline aqueous solution of one or more polyhydric alcohols. The product may be a complex mixture of polyethers with varying numbers of ether groups. Efficiency of the polyether cross-linking agent increases with the number of potentially polymerizable groups on the molecule. Typically, polyethers containing an average of two or more alkenyl ether groupings per molecule are used. Other cross-linking monomers include for example, diallyl esters, dimethallyl ethers, allyl or methallyl acrylates and acrylamides, tetravinyl silane, polyalkenyl methanes, diacrylates, and dimethacrylates, divinyl compounds such as divinyl benzene, polyallyl phosphate, diallyloxy compounds and phosphite esters and the like. Typical agents are allyl pentaerythritol, allyl sucrose, trimethylolpropane triacrylate, 1,6-hexanediol diacrylate, trimethylolpropane diallyl ether, pentaerythritol triacrylate, tetramethylene dimethacrylate, ethylene diacrylate, ethylene dimethacrylate, triethylene glycol dimethacrylate, and the like. Allyl pentaerythritol, trimethylolpropane diallylether and allyl sucrose provide suitable polymers. When the cross-linking agent is present, the polymeric mixtures usually contain between about 0.01 to 20 weight percent, e.g., 1%, 5%, or 10% or more by weight of cross-linking monomer based on the total of carboxylic acid monomer, plus other monomers.

In more detailed aspects of the invention, epithelial delivery of GRP, analogs and mimetics, and other biologically active agents disclosed herein, is enhanced by retaining the active agent(s) in a slow-release or enzymatically or physiologically protective carrier or vehicle, for example a hydrogel that shields the active agent from the action of the degradative enzymes. In certain embodiments, the active agent is bound by chemical means to the carrier or vehicle, to which may also be admixed or bound additional agents such as enzyme inhibitors, cytokines, etc. The active agent may alternately be immobilized through sufficient physical entrapment within the carrier or vehicle, e.g., a polymer matrix.

Polymers such as hydrogels useful within the invention may incorporate functional linked agents such as glycosides chemically incorporated into the polymer for enhancing intranasal bioavailability of active agents formulated therewith. Examples of such glycosides are glucosides, fructosides, galactosides, arabinosides, mannosides and their alkyl substituted derivatives and natural glycosides such as arbutin, phlorizin, amygdalin, digitonin, saponin, and indican. There are several ways in which a typical glycoside may be bound to a polymer. For example, the hydrogen of the hydroxyl groups of a glycoside or other similar carbohydrate may be replaced by the alkyl group from a hydrogel polymer to form an ether. Also, the hydroxyl groups of the glycosides may be reacted to esterify the carboxyl groups of a polymeric hydrogel to form polymeric esters in situ. Another approach is to employ condensation of acetobromoglucose with cholest-5-en-3beta-ol on a copolymer of maleic acid. N-substituted polyacrylamides can be synthesized by the reaction of activated polymers with omega-aminoalkylglycosides: (1) (carbohydrate-spacer)(n)-polyacrylamide, ‘pseudopolysaccharides’; (2) (carbohydrate spacer)(n)-phosphatidylethanolamine(m)-polyacrylamide, neoglycolipids, derivatives of phosphatidylethanolamine; (3) (carbohydrate-spacer)(n)-biotin(m)-polyacrylamide. These biotinylated derivatives may attach to lectins on the mucosal surface to facilitate absorption of the biologically active agent(s), e.g., a polymer-encapsulated GRP.

Within more detailed aspects of the invention, one or more GRP, analogs and mimetics, and/or other biologically active agents, disclosed herein, optionally including secondary active agents such as protease inhibitor(s), cytokine(s), additional modulator(s) of intercellular junctional physiology, etc., are modified and bound to a polymeric carrier or matrix. For example, this may be accomplished by chemically binding a peptide or protein active agent and other optional agent(s) within a crosslinked polymer network. It is also possible to chemically modify the polymer separately with an interactive agent such as a glycosidal containing molecule. In certain aspects, the biologically active agent(s), and optional secondary active agent(s), may be functionalized, i.e., wherein an appropriate reactive group is identified or is chemically added to the active agent(s). Most often an ethylenic polymerizable group is added, and the functionalized active agent is then copolymerized with monomers and a crosslinking agent using a standard polymerization method such as solution polymerization (usually in water), emulsion, suspension or dispersion polymerization. Often, the functionalizing agent is provided with a high enough concentration of functional or polymerizable groups to insure that several sites on the active agent(s) are functionalized. For example, in a polypeptide comprising 16 amine sites, it is generally desired to functionalize at least 2, 4, 5, 7, and up to 8 or more of the sites.

After functionalization, the functionalized active agent(s) is/are mixed with monomers and a crosslinking agent that comprise the reagents from which the polymer of interest is formed. Polymerization is then induced in this medium to create a polymer containing the bound active agent(s). The polymer is then combined with water or other appropriate solvents and otherwise purified to remove trace unreacted impurities and, if necessary, ground or broken up by physical means such as by stirring, forcing it through a mesh, ultrasonication or other suitable means to a desired particle size. The solvent, usually water, is then removed in such a manner as to not denature or otherwise degrade the active agent(s). One desired method is lyophilization (freeze drying) but other methods are available and may be used (e.g., vacuum drying, air drying, spray drying, etc.).

To introduce polymerizable groups in peptides, proteins and other active agents within the invention, it is possible to react available amino, hydroxyl, thiol and other reactive groups with electrophiles containing unsaturated groups. For example, unsaturated monomers containing N-hydroxy succinimidyl groups, active carbonates such as p-nitrophenyl carbonate, trichlorophenyl carbonates, tresylate, oxycarbonylimidazoles, epoxide, isocyanates and aldehyde, and unsaturated carboxymethyl azides and unsaturated orthopyridyl-disulfide belong to this category of reagents. Illustrative examples of unsaturated reagents are allyl glycidyl ether, allyl chloride, allylbromide, allyl iodide, acryloyl chloride, allyl isocyanate, allylsulfonyl chloride, maleic anhydride, copolymers of maleic anhydride and allyl ether, and the like.

All of the lysine active derivatives, except aldehyde, can generally react with other amino acids such as imidazole groups of histidine and hydroxyl groups of tyrosine and the thiol groups of cystine if the local environment enhances nucleophilicity of these groups. Aldehyde-containing functionalizing reagents are specific to lysine. These types of reactions with available groups from lysines, cysteines, tyrosine have been extensively documented in the literature and are known to those skilled in the art.

In the case of biologically active agents that contain amine groups, it is convenient to react such groups with an acyloyl chloride, such as acryloyl chloride, and introduce the polymerizable acrylic group onto the reacted agent. Then during preparation of the polymer, such as during the crosslinking of the copolymer of acrylamide and acrylic acid, the functionalized active agent, through the acrylic groups, is attached to the polymer and becomes bound thereto.

In additional aspects of the invention, biologically active agents, including peptides, proteins, nucleosides, and other molecules which are bioactive in vivo, are conjugation-stabilized by covalently bonding one or more active agent(s) to a polymer incorporating as an integral part thereof both a hydrophilic moiety, e.g., a linear polyalkylene glycol, a lipophilic moiety (see, e.g., U.S. Pat. No. 5,681,811). In one aspect, a biologically active agent is covalently coupled with a polymer comprising (i) a linear polyalkylene glycol moiety, and (ii) a lipophilic moiety, wherein the active agent, linear polyalkylene glycol moiety, and the lipophilic moiety are conformationally arranged in relation to one another such that the active therapeutic agent has an enhanced in vivo resistance to enzymatic degradation (i.e., relative to its stability under similar conditions in an unconjugated form devoid of the polymer coupled thereto). In another aspect, the conjugation-stabilized formulation has a three-dimensional conformation comprising the biologically active agent covalently coupled with a polysorbate complex comprising (i) a linear polyalkylene glycol moiety, and (ii) a lipophilic moiety, wherein the active agent, the linear polyalkylene glycol moiety and the lipophilic moiety are conformationally arranged in relation to one another such that (a) the lipophilic moiety is exteriorly available in the three-dimensional conformation, and (b) the active agent in the composition has an enhanced in vivo resistance to enzymatic degradation.

In a further related aspect, a multiligand conjugated complex is provided which comprises a biologically active agent covalently coupled with a triglyceride backbone moiety through a polyalkylene glycol spacer group bonded at a carbon atom of the triglyceride backbone moiety, and at least one fatty acid moiety covalently attached either directly to a carbon atom of the triglyceride backbone moiety or covalently joined through a polyalkylene glycol spacer moiety (see, e.g., U.S. Pat. No. 5,681,811). In such a multiligand conjugated therapeutic agent complex, the alpha′ and beta carbon atoms of the triglyceride bioactive moiety may have fatty acid moieties attached by covalently bonding either directly thereto, or indirectly covalently bonded thereto through polyalkylene glycol spacer moieties. Alternatively, a fatty acid moiety may be covalently attached either directly or through a polyalkylene glycol spacer moiety to the alpha and alpha′ carbons of the triglyceride backbone moiety, with the bioactive therapeutic agent being covalently coupled with the gamma-carbon of the triglyceride backbone moiety, either being directly covalently bonded thereto or indirectly bonded thereto through a polyalkylene spacer moiety. It will be recognized that a wide variety of structural, compositional, and conformational forms are possible for the multiligand conjugated therapeutic agent complex comprising the triglyceride backbone moiety, within the scope of the invention. It is further noted that in such a multiligand conjugated therapeutic agent complex, the biologically active agent(s) may advantageously be covalently coupled with the triglyceride modified backbone moiety through alkyl spacer groups, or alternatively other acceptable spacer groups, within the scope of the invention. As used in such context, acceptability of the spacer group refers to steric, compositional, and end use application specific acceptability characteristics.

In yet additional aspects of the invention, a conjugation-stabilized complex is provided which comprises a polysorbate complex comprising a polysorbate moiety including a triglyceride backbone having covalently coupled to alpha, alpha′ and beta carbon atoms thereof functionalizing groups including (i) a fatty acid group; and (ii) a polyethylene glycol group having a biologically active agent or moiety covalently bonded thereto, e.g., bonded to an appropriate functionality of the polyethylene glycol group. Such covalent bonding may be either direct, e.g., to a hydroxy terminal functionality of the polyethylene glycol group, or alternatively, the covalent bonding may be indirect, e.g., by reactively capping the hydroxy terminus of the polyethylene glycol group with a terminal carboxy functionality spacer group, so that the resulting capped polyethylene glycol group has a terminal carboxy functionality to which the biologically active agent or moiety may be covalently bonded.

In yet additional aspects of the invention, a stable, aqueously soluble, conjugation-stabilized complex is provided which comprises one or more GRP, analogs and mimetics, and/or other biologically active agent(s)+disclosed herein covalently coupled to a physiologically compatible polyethylene glycol (PEG) modified glycolipid moiety. In such complex, the biologically active agent(s) may be covalently coupled to the physiologically compatible PEG modified glycolipid moiety by a labile covalent bond at a free amino acid group of the active agent, wherein the labile covalent bond is scissionable in vivo by biochemical hydrolysis and/or proteolysis. The physiologically compatible PEG modified glycolipid moiety may advantageously comprise a polysorbate polymer, e.g., a polysorbate polymer comprising fatty acid ester groups selected from the group consisting of monopalmitate, dipalmitate, monolaurate, dilaurate, trilaurate, monoleate, dioleate, trioleate, monostearate, distearate, and tristearate. In such complex, the physiologically compatible PEG modified glycolipid moiety may suitably comprise a polymer selected from the group consisting of polyethylene glycol ethers of fatty acids, and polyethylene glycol esters of fatty acids, wherein the fatty acids for example comprise a fatty acid selected from the group consisting of lauric, palmitic, oleic, and stearic acids.

### Storage of Material

In certain aspects of the invention, the combinatorial formulations and/or coordinate administration methods herein incorporate an effective amount of peptides and proteins which may adhere to charged glass thereby reducing the effective concentration in the container. Silanized containers, for example, silanized glass containers, are used to store the finished product to reduce adsorption of the polypeptide or protein to a glass container.

In yet additional aspects of the invention, a kit for treatment of a mammalian subject comprises a stable pharmaceutical composition of one or more GRP compound(s) formulated for mucosal delivery to the mammalian subject wherein the composition is effective to alleviate one or more symptom(s) of diabetes, obesity, cancer, hyperglycemia, dyslipidemia, metabolic syndrome, coronary syndrome, myocardial infraction, or neurological disorder in said subject without unacceptable adverse side effects. The kit further comprises a pharmaceutical reagent vial to contain the one or more GRP compounds. The pharmaceutical reagent vial is composed of pharmaceutical grade polymer, glass or other suitable material. The pharmaceutical reagent vial is, for example, a silanized glass vial. The kit further comprises an aperture for delivery of the composition to a nasal mucosal surface of the subject. The delivery aperture is composed of a pharmaceutical grade polymer, glass or other suitable material. The delivery aperture is, for example, a silanized glass.

A silanization technique combines a special cleaning technique for the surfaces to be silanized with a silanization process at low pressure. The silane is in the gas phase and at an enhanced temperature of the surfaces to be silanized. The method provides reproducible surfaces with stable, homogeneous and functional silane layers having characteristics of a monolayer. The silanized surfaces prevent binding to the glass of polypeptides or mucosal delivery enhancing agents of the present invention.

The procedure is useful to prepare silanized pharmaceutical reagent vials to hold GRP compositions of the present invention. Glass trays are cleaned by rinsing with double distilled water (ddH₂O) before using. The silane tray is then be rinsed with 95% EtOH, and the acetone tray is rinsed with acetone. Pharmaceutical reagent vials are sonicated in acetone for 10 minutes. After the acetone sonication, reagent vials are ished in ddH₂O tray at least twice. Reagent vials are sonicated in 0.1M NaOH for 10 minutes. While the reagent vials are sonicating in NaOH, the silane solution is made under a hood. (Silane solution: 800 mL of 95% ethanol; 96 L of glacial acetic acid; 25 mL of glycidoxypropyltrimethoxy silane). After the NaOH sonication, reagent vials are ished in ddH₂O tray at least twice. The reagent vials are sonicated in silane solution for 3 to 5 minutes. The reagent vials are ished in 100% EtOH tray. The reagent vials are dried with prepurified N₂ gas and stored in a 100° C. oven for at least 2 hours before using.

### Bioadhesive Delivery Vehicles and Methods

In certain aspects of the invention, the combinatorial formulations and/or coordinate administration methods herein incorporate an effective amount of a nontoxic bioadhesive as an adjunct compound or carrier to enhance epithelial delivery of one or more biologically active agent(s). Bioadhesive agents in this context exhibit general or specific adhesion to one or more components or surfaces of the targeted biological membrane. The bioadhesive maintains a desired concentration gradient of the biologically active agent into or across the mucosa to ensure penetration of even large molecules (e.g., peptides and proteins) into or through the epithelium. Typically, employment of a bioadhesive within the methods and compositions of the invention yields a two- to five-fold, often a five- to ten-fold increase in permeability for peptides and proteins into or through the epithelium. This enhancement of epithelial permeation often permits effective transmucosal delivery of large macromolecules, for example to the basal portion of the nasal epithelium or into the adjacent extracellular compartments or a blood plasma or CNS tissue or fluid.

This enhanced delivery provides for greatly improved effectiveness of delivery of bioactive peptides, proteins and other macromolecular therapeutic species. These results will depend in part on the hydrophilicity of the compound, whereby greater penetration will be achieved with hydrophilic species compared to water insoluble compounds. In addition to these effects, employment of bioadhesives to enhance drug persistence at the biological membrane surface can elicit a reservoir mechanism for protracted drug delivery, whereby compounds not only penetrate across the biological membrane but also back-diffuse toward the surface once the material at the surface is depleted.

A variety of suitable bioadhesives are disclosed in the art for oral administration, U.S. Pat. Nos. 3,972,995; 4,259,314; 4,680,323; 4,740,365; 4,573,996; 4,292,299; 4,715,369; 4,876,092; 4,855,142; 4,250,163; 4,226,848; 4,948,580; U.S. Pat. Reissue No. 33,093, which find use within the novel methods and compositions of the invention. The potential of various bioadhesive polymers as a mucosal, e.g., nasal, delivery platform within the methods and compositions of the invention can be readily assessed by determining their ability to retain and release GRP, as well as by their capacity to interact with the mucosal surfaces following incorporation of the active agent therein. In addition, well known methods will be applied to determine the biocompatibility of selected polymers with the tissue at the site of mucosal administration. When the target mucosa is covered by mucus (i.e., in the absence of mucolytic or mucus-clearing treatment), it can serve as a connecting link to the underlying mucosal epithelium. Therefore, the term “bioadhesive” as used herein also covers mucoadhesive compounds useful for enhancing mucosal delivery of biologically active agents within the invention. However, adhesive contact to mucosal tissue mediated through adhesion to a mucus gel layer may be limited by incomplete or transient attachment between the mucus layer and the underlying tissue, particularly at nasal surfaces where rapid mucus clearance occurs. In this regard, mucin glycoproteins are continuously secreted and, immediately after their release from cells or glands, form a viscoelastic gel. The luminal surface of the adherent gel layer, however, is continuously eroded by mechanical, enzymatic and/or ciliary action. Where such activities are more prominent or where longer adhesion times are desired, the coordinate administration methods and combinatorial formulation methods of the invention may further incorporate mucolytic and/or ciliostatic methods or agents as disclosed herein above.

Typically, mucoadhesive polymers for use within the invention are natural or synthetic macromolecules which adhere to wet mucosal tissue surfaces by complex, but non-specific, mechanisms. In addition to these mucoadhesive polymers, the invention also provides methods and compositions incorporating bioadhesives that adhere directly to a cell surface, rather than to mucus, by means of specific, including receptor-mediated, interactions. One example of bioadhesives that function in this specific manner is the group of compounds known as lectins. These are glycoproteins with an ability to specifically recognize and bind to sugar molecules, e.g. glycoproteins or glycolipids, which form part of intranasal epithelial cell membranes and can be considered as “lectin receptors.”

In certain aspects of the invention, bioadhesive materials for enhancing intranasal delivery of biologically active agents comprise a matrix of a hydrophilic, e.g., water soluble or swellable, polymer or a mixture of polymers that can adhere to a wet mucous surface. These adhesives may be formulated as ointments, hydrogels (see above) thin films, and other application forms. Often, these adhesives have the biologically active agent mixed therewith to effectuate slow release or local delivery of the active agent. Some are formulated with additional ingredients to facilitate penetration of the active agent through the nasal mucosa, e.g., into the circulatory system of the individual.

Various polymers, both natural and synthetic ones, show significant binding to mucus and/or mucosal epithelial surfaces under physiological conditions. The strength of this interaction can readily be measured by mechanical peel or shear tests. When applied to a humid mucosal surface, many dry materials will spontaneously adhere, at least slightly. After such an initial contact, some hydrophilic materials start to attract water by adsorption, swelling or capillary forces, and if this water is absorbed from the underlying substrate or from the polymer-tissue interface, the adhesion may be sufficient to achieve the goal of enhancing mucosal absorption of biologically active agents. Such ‘adhesion by hydration’ can be quite strong, but formulations adapted to employ this mechanism must account for swelling which continues as the dosage transforms into a hydrated mucilage. This is projected for many hydrocolloids useful within the invention, especially some cellulose-derivatives, which are generally non-adhesive when applied in pre-hydrated state. Nevertheless, bioadhesive drug delivery systems for epithelial administration are effective within the invention when such materials are applied in the form of a dry polymeric powder, microsphere, or film-type delivery form.

Other polymers adhere to epithelial surfaces not only when applied in dry, but also in fully hydrated state, and in the presence of excess amounts of water. The selection of a mucoadhesive thus requires due consideration of the conditions, physiological as well as physico-chemical, under which the contact to the tissue will be formed and maintained. In particular, the amount of water or humidity usually present at the intended site of adhesion, and the prevailing pH, are known to largely affect the mucoadhesive binding strength of different polymers.

Several polymeric bioadhesive drug delivery systems have been fabricated and studied in the past 20 years, not always with success. A variety of such carriers are, however, currently used in clinical applications involving dental, orthopedic, opthalmological, and surgical uses. For example, acrylic-based hydrogels have been used extensively for bioadhesive devices. Acrylic-based hydrogels are well suited for bioadhesion due to their flexibility and nonabrasive characteristics in the partially swollen state, which reduce damage-causing attrition to the tissues in contact. Furthermore, their high permeability in the swollen state allows unreacted monomer, un-crosslinked polymer chains, and the initiator to be ished out of the matrix after polymerization, which is an important feature for selection of bioadhesive materials for use within the invention. Acrylic-based polymer devices exhibit very high adhesive bond strength. For controlled epithelial delivery of peptide and protein drugs, the methods and compositions of the invention optionally include the use of carriers, e.g., polymeric delivery vehicles, that function in part to shield the biologically active agent from proteolytic breakdown, while at the same time providing for enhanced penetration of the peptide or protein into or through the nasal mucosa. In this context, bioadhesive polymers have demonstrated considerable potential for enhancing oral drug delivery. As an example, the bioavailability of 9-desglycinamide, 8-arginine vasopressin (DGAVP) intraduodenally administered to rats together with a 1% (w/v) saline dispersion of the mucoadhesive poly(acrylic acid) derivative polycarbophil, is 3-5-fold increased compared to an aqueous solution of the peptide drug without this polymer.

Mucoadhesive polymers of the poly(acrylic acid)-type are potent inhibitors of some intestinal proteases. The mechanism of enzyme inhibition is explained by the strong affinity of this class of polymers for divalent cations, such as calcium or zinc, which are essential cofactors of metallo-proteinases, such as trypsin and chymotrypsin. Depriving the proteases of their cofactors by poly(acrylic acid) is reported to induce irreversible structural changes of the enzyme proteins which were accompanied by a loss of enzyme activity. At the same time, other mucoadhesive polymers (e.g., some cellulose derivatives and chitosan) may not inhibit proteolytic enzymes under certain conditions. In contrast to other enzyme inhibitors contemplated for use within the invention (e.g. aprotinin, bestatin), which are relatively small molecules, the trans-nasal absorption of inhibitory polymers is likely to be minimal in light of the size of these molecules, and thereby eliminate possible adverse side effects. Thus, mucoadhesive polymers, particularly of the poly(acrylic acid)-type, may serve both as an absorption-promoting adhesive and enzyme-protective agent to enhance controlled delivery of peptide and protein drugs, especially when safety concerns are considered.

In addition to protecting against enzymatic degradation, bioadhesives and other polymeric or non-polymeric absorption-promoting agents for use within the invention may directly increase epithelial permeability to biologically active agents. To facilitate the transport of large and hydrophilic molecules, such as peptides and proteins, across the nasal epithelial barrier, mucoadhesive polymers and other agents have been postulated to yield enhanced permeation effects beyond what is accounted for by prolonged premucosal residence time of the delivery system. The time course of drug plasma concentrations reportedly suggested that the bioadhesive microspheres caused an acute, but transient increase of insulin permeability across the nasal mucosa. Other mucoadhesive polymers for use within the invention, for example chitosan, reportedly enhance the permeability of certain mucosal epithelia even when they are applied as an aqueous solution or gel. Another mucoadhesive polymer reported to directly affect epithelial permeability is hyaluronic acid and ester derivatives thereof. A particularly useful bioadhesive agent within the coordinate administration, and/or combinatorial formulation methods and compositions of the invention is chitosan, as well as its analogs and derivatives. Chitosan is a non-toxic, biocompatible and biodegradable polymer that is widely used for pharmaceutical and medical applications because of its favorable properties of low toxicity and good biocompatibility. It is a natural polyaminosaccharide prepared from chitin by N-deacetylation with alkali. As used within the methods and compositions of the invention, chitosan increases the retention of GRP, analogs and mimetics, and other biologically active agents disclosed herein at a mucosal site of application. This mode of administration can also improve patient compliance and acceptance. As further provided herein, the methods and compositions of the invention will optionally include a novel chitosan derivative or chemically modified form of chitosan. One such novel derivative for use within the invention is denoted as a β-[1→4]-2-guanidino-2-deoxy-D-glucose polymer (poly-GuD). Chitosan is the N-deacetylated product of chitin, a naturally occurring polymer that has been used extensively to prepare microspheres for oral and intra-nasal formulations. The chitosan polymer has also been proposed as a soluble carrier for parenteral drug delivery. Within one aspect of the invention, o-methylisourea is used to convert a chitosan amine to its guanidinium moiety. The guanidinium compound is prepared, for example, by the reaction between equi-normal solutions of chitosan and o-methylisourea at pH above 8.0.

Additional compounds classified as bioadhesive agents for use within the present invention act by mediating specific interactions, typically classified as “receptor-ligand interactions” between complementary structures of the bioadhesive compound and a component of the mucosal epithelial-surface. Many natural examples illustrate this form of specific binding bioadhesion, as exemplified by lectin-sugar interactions. Lectins are (glyco) proteins of non-immune origin which bind to polysaccharides or glycoconjugates.

Several plant lectins have been investigated as possible pharmaceutical absorption-promoting agents. One plant lectin, Phaseolus vulgaris hemagglutinin (PHA), exhibits high oral bioavailability of more than 10% after feeding to rats. Tomato (Lycopersicon esculeutum) lectin (TL) appears safe for various modes of administration.

In summary, the foregoing bioadhesive agents are useful in the combinatorial formulations and coordinate administration methods of the instant invention, which optionally incorporate an effective amount and form of a bioadhesive agent to prolong persistence or otherwise increase epithelial absorption of one or more GRP, analogs and mimetics, and other biologically active agents. The bioadhesive agents may be coordinately administered as adjunct compounds or as additives within the combinatorial formulations of the invention. In certain embodiments, the bioadhesive agent acts as a ‘pharmaceutical glue,’ whereas in other embodiments adjunct delivery or combinatorial formulation of the bioadhesive agent serves to intensify contact of the biologically active agent with the nasal mucosa, in some cases by promoting specific receptor-ligand interactions with epithelial cell “receptors,” and in others by increasing epithelial permeability to significantly increase the drug concentration gradient measured at a target site of delivery (e.g., liver, blood plasma, or CNS tissue or fluid). Yet additional bioadhesive agents for use within the invention act as enzyme (e.g., protease) inhibitors to enhance the stability of mucosally administered biotherapeutic agents delivered coordinately or in a combinatorial formulation with the bioadhesive agent.

### Liposomes and Micellar Delivery Vehicles

The coordinate administration methods and combinatorial formulations of the instant invention optionally incorporate effective lipid or fatty acid based carriers, processing agents, or delivery vehicles, to provide improved formulations for epithelial delivery of GRP, analogs and mimetics, and other biologically active agents. For example, a variety of formulations and methods are provided for mucosal delivery which comprise one or more of these active agents, such as a peptide or protein, admixed or encapsulated by, or coordinately administered with, a liposome, mixed micellar carrier, or emulsion, to enhance chemical and physical stability and increase the half life of the biologically active agents (e.g., by reducing susceptibility to proteolysis, chemical modification and/or denaturation) upon mucosal delivery.

Within certain aspects of the invention, specialized delivery systems for biologically active agents comprise small lipid vesicles known as liposomes. These are typically made from natural, biodegradable, non-toxic, and non-immunogenic lipid molecules, and can efficiently entrap or bind drug molecules, including peptides and proteins, into, or onto, their membranes. The attractiveness of liposomes as a peptide and protein delivery system within the invention is increased by the fact that the encapsulated proteins can remain in their preferred aqueous environment within the vesicles, while the liposomal membrane protects them against proteolysis and other destabilizing factors. Even though not all liposome preparation methods known are feasible in the encapsulation of peptides and proteins due to their unique physical and chemical properties, several methods allow the encapsulation of these macromolecules without substantial deactivation.

A variety of methods are available for preparing liposomes for use within the invention, U.S. Pat. Nos. 4,235,871, 4,501,728, and 4,837,028. For use with liposome delivery, the biologically active agent is typically entrapped within the liposome, or lipid vesicle, or is bound to the outside of the vesicle.

Like liposomes, unsaturated long chain fatty acids, which also have enhancing activity for mucosal absorption, can form closed vesicles with bilayer-like structures (so called “ufasomes”). These can be formed, for example, using oleic acid to entrap biologically active peptides and proteins for mucosal, e.g., intranasal, delivery within the invention.

Other delivery systems for use within the invention combine the use of polymers and liposomes to ally the advantageous properties of both vehicles such as encapsulation inside the natural polymer fibrin. In addition, release of biotherapeutic compounds from this delivery system is controllable through the use of covalent crosslinking and the addition of antifibrinolytic agents to the fibrin polymer.

More simplified delivery systems for use within the invention include the use of cationic lipids as delivery vehicles or carriers, which can be effectively employed to provide an electrostatic interaction between the lipid carrier and such charged biologically active agents as proteins and polyanionic nucleic acids. This allows efficient packaging of the drugs into a form suitable for mucosal administration and/or subsequent delivery to systemic compartments.

Additional delivery vehicles for use within the invention include long and medium chain fatty acids, as well as surfactant mixed micelles with fatty acids. Most naturally occurring lipids in the form of esters have important implications with regard to their own transport across mucosal surfaces. Free fatty acids and their monoglycerides which have polar groups attached have been demonstrated in the form of mixed micelles to act on the intestinal barrier as penetration enhancers. This discovery of barrier modifying function of free fatty acids (carboxylic acids with a chain length varying from 12 to 20 carbon atoms) and their polar derivatives has stimulated extensive research on the application of these agents as mucosal absorption enhancers.

For use within the methods of the invention, long chain fatty acids, especially fusogenic lipids (unsaturated fatty acids and monoglycerides such as oleic acid, linoleic acid, linoleic acid, monoolein, etc.) provide useful carriers to enhance mucosal delivery of GRP, analogs and mimetics, and other biologically active agents disclosed herein. Medium chain fatty acids (C6 to C12) and monoglycerides have also been shown to have enhancing activity in intestinal drug absorption and can be adapted for use within the mocosal delivery formulations and methods of the invention. In addition, sodium salts of medium and long chain fatty acids are effective delivery vehicles and absorption-enhancing agents for epithelial delivery of biologically active agents within the invention. Thus, fatty acids can be employed in soluble forms of sodium salts or by the addition of non-toxic surfactants, e.g., polyoxyethylated hydrogenated castor oil, sodium taurocholate, etc. Other fatty acid and mixed micellar preparations that are useful within the invention include, but are not limited to, Na caprylate (C8), Na caprate (C10), Na laurate (C12) or Na oleate (C18), optionally combined with bile salts, such as glycocholate and taurocholate.

### Pegylation

Additional methods and compositions provided within the invention involve chemical modification of biologically active peptides and proteins by covalent attachment of polymeric materials, for example dextrans, polyvinyl pyrrolidones, glycopeptides, polyethylene glycol and polyamino acids. The resulting conjugated peptides and proteins retain their biological activities and solubility for epithelial administration. In alternate embodiments, GRP, analogs and mimetics, and other biologically active peptides and proteins, are conjugated to polyalkylene oxide polymers, particularly polyethylene glycols (PEG). U.S. Pat. No. 4,179,337.

Amine-reactive PEG polymers for use within the invention include SC-PEG with molecular masses of 2000, 5000, 10000, 12000, and 20 000; U-PEG-10000; NHS-PEG-3400-biotin; T-PEG-5000; T-PEG-12000; and TPC-PEG-5000. PEGylation of biologically active peptides and proteins may be achieved by modification of carboxyl sites (e.g., aspartic acid or glutamic acid groups in addition to the carboxyl terminus). The utility of PEG-hydrazide in selective modification of carbodiimide-activated protein carboxyl groups under acidic conditions has been described. Alternatively, bifunctional PEG modification of biologically active peptides and proteins can be employed. In some procedures, charged amino acid residues, including lysine, aspartic acid, and glutamic acid, have a marked tendency to be solvent accessible on protein surfaces.

### Other Stabilizing Modifications of Active Agents

In addition to PEGylation, biologically active agents such as peptides and proteins for use within the invention can be modified to enhance circulating half-life by shielding the active agent via conjugation to other known protecting or stabilizing compounds, for example by the creation of fusion proteins with an active peptide, protein, analog or mimetic linked to one or more carrier proteins, such as one or more immunoglobulin chains.

### Formulation Preparation, Manufacture, and Administration

Epithelial delivery formulations of the present invention comprise GRP, analogs and mimetics, typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

Within the compositions and methods of the invention, the GRP, analogs and mimetics, and other biologically active agents disclosed herein may be administered to subjects by a variety of mucosal administration modes, including by oral, rectal, vaginal, intranasal, intrapulmonary, or transdermal delivery, or by topical delivery to the eyes, ears, skin or other mucosal surfaces. The compositions and methods of the invention also include dermal administration modes. Optionally, GRP, analogs and mimetics, and other biologically active agents disclosed herein can be coordinately or adjunctively administered by non-mucosal routes, including by dermal patch, topical preparation applied to the skin, intramuscular, subcutaneous, intravenous, intra-atrial, intra-articular, intraperitoneal, or parenteral routes. In other alternative embodiments, the biologically active agent(s) can be administered ex vivo by direct exposure to cells, tissues or organs originating from a mammalian subject, for example as a component of an ex vivo tissue or organ treatment formulation that contains the biologically active agent in a suitable, liquid or solid carrier.

Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-,jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present invention typically comprise the drug or drug to be delivered, optionally formulated with a surface-active agent, such as a nonionic surfactant (e.g., polysorbate-80), and one or more buffers. In some embodiments of the present invention, the nasal spray solution further comprises a propellant. The pH of the nasal spray solution is optionally between about pH 2.0 and 8, preferably 4.5±0.5. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, sodium benzoate, ethanol, phenylethyl ether, benzyl alcohol and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, mucosal formulations are administered as dry powder formulations comprising the biologically active agent in a dry, usually lyophilized, form of an appropriate particle size, or within an appropriate particle size range, for intranasal delivery. A dry formulation may also be appropriate for dermal delivery. Minimum particle size appropriate for deposition within the nasal or pulmonary passages is often about 0.5μ mass median equivalent aerodynamic diameter (MMEAD), commonly about 1μ MMEAD, and more typically about 2μ MMEAD. Maximum particle size appropriate for deposition within the nasal passages is often about 10μ MMEAD, commonly about 8μ MMEAD, and more typically about 4μ MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which rely on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

Dry powder devices typically require a powder mass in the range from about 1 mg to 20 mg to produce a single aerosolized dose (“puff”). If the required or desired dose of the biologically active agent is lower than this amount, the powdered active agent will typically be combined with a pharmaceutical dry bulking powder to provide the required total powder mass. Preferred dry bulking powders include sucrose, lactose, dextrose, mannitol, glycine, trehalose, human serum albumin (HSA), and starch. Other suitable dry bulking powders include cellobiose, dextrans, maltotriose, pectin, sodium citrate, sodium ascorbate, and the like.

To formulate compositions for epithelial delivery within the present invention, the biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, acetic acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for epithelial delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g. maleic anhydride) with other monomers (e.g. methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate and dispersed in a biocompatible dispersing medium applied to the nasal mucosa, which yields sustained delivery and biological activity over a protracted time.

To further enhance epithelial delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10000 and preferably not more than 3000. Exemplary hydrophilic low molecular weight compound include polyol compounds, such as oligo-, di- and monosaccarides such as sucrose, mannitol, sorbitol, lactose, L-arabinose, D-erythrose, D-ribose, D-xylose, D-mannose, trehalose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g. oligovinyl alcohol, ethanol, ethylene glycol, propylene glycol, etc.) These hydrophilic low molecular weight compounds can be used alone or in combination with one another or with other active or inactive components of the intranasal formulation.

The compositions of the invention may alternatively contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

Therapeutic compositions for administering the biologically active agent can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols such as mannitol, sorbitol, or sodium chloride in the composition. Prolonged absorption of the biologically active agent can be brought about by including in the composition an agent which delays absorption, for example, monostearate salts and gelatin.

In certain embodiments of the invention, the biologically active agent is administered in a time-release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions of the invention can be brought about by including in the composition agents that delay absorption, for example, aluminum monosterate hydrogels and gelatin. When controlled release formulations of the biologically active agent is desired, controlled release binders suitable for use in accordance with the invention include any biocompatible controlled-release material which is inert to the active agent and which is capable of incorporating the biologically active agent. Numerous such materials are known in the art. Useful controlled-release binders are materials that are metabolized slowly under physiological conditions following their intranasal delivery (e.g., at the nasal mucosal surface, or in the presence of bodily fluids following transmucosal delivery). Appropriate binders include but are not limited to biocompatible polymers and copolymers previously used in the art in sustained release formulations. Such biocompatible compounds are non-toxic and inert to surrounding tissues, and do not trigger significant adverse side effects such as nasal irritation, immune response, inflammation, or the like. They are metabolized into metabolic products that are also biocompatible and easily eliminated from the body.

Exemplary polymeric materials for use in this context include, but are not limited to, polymeric matrices derived from copolymeric and homopolymeric polyesters having hydrolysable ester linkages. A number of these are known in the art to be biodegradable and to lead to degradation products having no or low toxicity. Exemplary polymers include polyglycolic acids (PGA) and polylactic acids (PLA), poly(DL-lactic acid-co-glycolic acid) (DL PLGA), poly(D-lactic acid-coglycolic acid) (D PLGA) and poly(L-lactic acid-co-glycolic acid) (L PLGA). Other useful biodegradable or bioerodable polymers include but are not limited to such polymers as poly(epsilon-caprolactone), poly(epsilon-aprolactone-CO-lactic acid), poly(s-aprolactone-CO-glycolic acid), poly(beta-hydroxy butyric acid), poly(alkyl-2-cyanoacrilate), hydrogels such as poly(hydroxyethyl methacrylate), polyamides, poly(amino acids) (i.e., L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides and copolymers thereof. Many methods for preparing such formulations are generally known to those skilled in the art. Other useful formulations include controlled-release compositions e.g., microcapsules, U.S. Pat. Nos. 4,652,441 and 4,917,893, lactic acid-glycolic acid copolymers useful in making microcapsules and other formulations, U.S. Pat. Nos. 4,677,191 and 4,728,721, and sustained-release compositions for water-soluble peptides, U.S. Pat. No. 4,675,189.

The nasal spray product manufacturing process generally includes the preparation of a diluent for GRP nasal spray, which includes ˜85% water plus the components of the nasal spray formulation without GRP. The pH of the diluent is then measured and adjusted to the desired formulation pH with sodium hydroxide or hydrochloric acid, if necessary. Water is used to achieve to the final target volume of diluent. The GRP nasal spray is prepared by the non-aseptic transfer of ˜85% of the final target volume of the diluent to a screw cap bottle. An appropriate amount of GRP is added and mixed until completely dissolved. The pH is measured and adjusted to the desired formulation pH with sodium hydroxide or hydrochloric acid, if necessary. A sufficient quantity of diluent is added to reach the final target volume. Screw-cap bottles are filled and caps affixed. The above description of the manufacturing process represents a method used to prepare the initial clinical batches of drug product. This method may be modified during the development process to optimize the manufacturing process.

Currently marketed GRP requires sterile manufacturing conditions for compliance with FDA regulations. Parenteral administration, including GRP for injection or infusion, requires a sterile (aseptic) manufacturing process. Current Good Manufacturing Practices (GMP) for sterile drug manufacturing include standards for design and construction features (21 C.F.R. § 211.42 (Apr. 1, 2005)); standards for testing and approval or rejection of components, drug product containers, and closures (§ 211.84); standards for control of microbiological contamination (§ 211.113); and other special testing requirements (§ 211.167). Non-parenteral (non-aseptic) products, such as the intranasal product of the invention, do not require these specialized sterile manufacturing conditions. As can be readily appreciated, the requirements for a sterile manufacturing process are substantially higher and correspondingly more costly than those required for a non-sterile product manufacturing process. These costs include much greater capitalization costs for facilities, as well as a more costly manufacturing cost: extra facilites for sterile manufacturing include additional rooms and ventilation; extra costs associated with sterile manufacturing include greater manpower, extensive quality control and quality assurance, and administrative support. As a result, manufacturing costs of an intranasal GRP product, such as that of the invention, are far less than those of a parenterally administered GRP product. The present invention satisfies the need for a non-sterile manufacturing process for GRP.

The invention includes a preservative-free GRP drug product. Such a formulation does not contain a preservative. In the absence of an antimicrobial excipient, the formulation would be filled under sterile conditions into a preservative-free nasal spray device or incorporated into a dermal patch preparation. The device would be capable of delivering an effective dose without allowing contamination of the formulation inside the delivery system. Such GRP drug product would allow for multi-dosing from the same container, thereby greatly reducing the cost of goods relative to a single-use drug product. Advantages of a multi-use preservative-free GRP formulation are improved stability, alternative means for prevention of microbial contamination, and reduction in the cost of goods allowing the product to be more viable for commercialization.

Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders, methods of preparation include vacuum drying and freeze-drying which yields a powder of the active ingredient plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

Mucosal and skin administration according to the invention allows effective self-administration of treatment by patients, provided that sufficient safeguards are in place to control and monitor dosing and side effects. Mucosal and skin administration also overcomes certain drawbacks of other administration forms, such as injections, that are painful and expose the patient to possible infections and may present drug bioavailability problems. For nasal and pulmonary delivery, systems for controlled aerosol dispensing of therapeutic liquids as a spray are well known. In one embodiment, metered doses of active agent are delivered by means of a specially constructed mechanical pump valve, U.S. Pat. No. 4,511,069. In another embodiment, active agent is delivered by dermal patch technology.

### Dosage

For prophylactic and treatment purposes, the biologically active agent(s) disclosed herein may be administered to the subject in a single bolus delivery, via continuous delivery (e.g., continuous transdermal, mucosal, or intravenous delivery) over an extended time period, or in a repeated administration protocol (e.g., by an hourly, daily or weekly, repeated administration protocol). In this context, a therapeutically effective dosage of the GRP may include repeated doses within a prolonged prophylaxis or treatment regimen that will yield clinically significant results to alleviate one or more symptoms or detectable conditions associated with a targeted disease or condition as set forth above. Determination of effective dosages in this context is typically based on animal model studies followed up by human clinical trials and is guided by determining effective dosages and administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

In an alternative embodiment, the invention provides compositions and methods for intranasal delivery of GRP, wherein the GRP compound(s) is/are repeatedly administered through an intranasal effective dosage regimen that involves multiple administrations of the GRP to the subject during a daily or weekly schedule to maintain a therapeutically effective elevated and lowered pulsatile level of GRP during an extended dosing period. The compositions and method provide GRP compound(s) that are self-administered by the subject in a nasal formulation between one and six times daily to maintain a therapeutically effective elevated and lowered pulsatile level of GRP during an 8 hour to 24 hour extended dosing period.

### Kits

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains one or more GRP, analogs or mimetics, and/or other biologically active agents in combination with epithelial delivery enhancing agents disclosed herein formulated in a pharmaceutical preparation for epithelial delivery.

The intranasal formulations of the present invention can be administered using any spray bottle or syringe, or by instillation. An example of a nasal spray bottle is the, “Nasal Spray Pump w/Safety Clip, Pfeiffer SAP # 60548, which delivers a dose of 0.1 mL per squirt and has a diptube length of 36.05 mm. It can be purchased from Pfeiffer of America of Princeton, N.J.

### Aerosol Nasal Administration of a GRP

We have discovered that the GRPs can be administered intranasally using a nasal spray or aerosol. This is surprising because many proteins and peptides have been shown to be sheared or denatured due to the mechanical forces generated by the actuator in producing the spray or aerosol. In this area the following definitions are useful:

1. Aerosol—A product that is packaged under pressure and contains therapeutically active ingredients that are released upon activation of an appropriate valve system.

2. Metered aerosol—A pressurized dosage form comprised of metered dose valves, which allow for the delivery of a uniform quantity of spray upon each activation.

3. Powder aerosol—A product that is packaged under pressure and contains therapeutically active ingredients in the form of a powder, which are released upon activation of an appropriate valve system.

4. Spray aerosol—An aerosol product that utilizes a compressed gas as the propellant to provide the force necessary to expel the product as a wet spray; it generally applicable to solutions of medicinal agents in aqueous solvents.

5. Spray—A liquid minutely divided as by a jet of air or steam. Nasal spray drug products contain therapeutically active ingredients dissolved or suspended in solutions or mixtures of excipients in nonpressurized dispensers.

6. Metered spray—A non-pressurized dosage form consisting of valves that allow the dispensing of a specified quantity of spray upon each activation.

7. Suspension spray—A liquid preparation containing solid particles dispersed in a liquid vehicle and in the form of course droplets or as finely divided solids.

The fluid dynamic characterization of the aerosol spray emitted by metered nasal spray pumps as a drug delivery device (“DDD”). Spray characterization is an integral part of the regulatory submissions necessary for Food and Drug Administration (“FDA”) approval of research and development, quality assurance and stability testing procedures for new and existing nasal spray pumps.

Thorough characterization of the spray's geometry has been found to be the best indicator of the overall performance of nasal spray pumps. In particular, measurements of the spray's divergence angle (plume geometry) as it exits the device; the spray's cross-sectional ellipticity, uniformity and particle/droplet distribution (spray pattern); and the time evolution of the developing spray have been found to be the most representative performance quantities in the characterization of a nasal spray pump. During quality assurance and stability testing, plume geometry and spray pattern measurements are key identifiers for verifying consistency and conformity with the approved data criteria for the nasal spray pumps.

### DEFINITIONS

Plume Height—the measurement from the actuator tip to the point at which the plume angle becomes non-linear because of the breakdown of linear flow. Based on a visual examination of digital images, and to establish a measurement point for width that is consistent with the farthest measurement point of spray pattern, a height of 30 mm is defined for this study

Major Axis—the largest chord that can be drawn within the fitted spray pattern that crosses the COMw in base units (mm)

Minor Axis—the smallest chord that can be drawn within the fitted spray pattern that crosses the COMw in base units (mm)

Ellipticity Ratio—the ratio of the major axis to the minor axis, preferably between 1.0 and 1.5, and most preferably between 1.0 and 1.3.

D₁₀—the diameter of droplet for which 10% of the total liquid volume of sample consists of droplets of a smaller diameter (μm)

D₅₀—the diameter of droplet for which 50% of the total liquid volume of sample consists of droplets of a smaller diameter (μm), also known as the mass median diameter

D₉₀—the diameter of droplet for which 90% of the total liquid volume of sample consists of droplets of a smaller diameter (μm)

Span—measurement of the width of the distribution, The smaller the value, the narrower the distribution. Span is calculated as

$$\frac{( {D}_{90} - {D}_{10} )}{{D}_{50}} .$$

% RSD—percent relative standard deviation, the standard deviation divided by the mean of the series and multiplied by 100, also known as % CV.

Volume—the volume of liquid or powder discharged from the delivery device with each actuation, preferably between 0.01 mL and about 2.5 mL and most preferably between 0.02 mL and 0.25 mL.

### EXAMPLES

The above disclosure generally describes the present invention, which is further exemplified by the following examples. These examples are described solely for purposes of illustration, and are not intended to limit the scope of the invention. Although specific terms and values have been employed herein, such terms and values will likewise be understood as exemplary and non-limiting to the scope of the invention.

### Example 1

### Materials and Equipment Used

The present example illustrates the reagents, equipment and the source of each used in the subsequent Examples of the instant application. Table 1 illustrates the sample reagents used in the subsequent Examples.

TABLE 1
GLP-1 Sample Reagents

| Reagent | Grade | Vendor | Cat # | Lot # | F.W. |
|---|---|---|---|---|---|
| GLP-1 (7-36 amide) | GMP | Bachem | H-6795.1000 | FCLP0401A | 3298 |
| Sodium Citrate | USP | Spectrum | S0165 | TE0713 | 294.10 |
| Citric Acid | USP | Sigma | C-1857 | 073K0061 | 192.13 |
| Methyl-β-cyclodextrin | | CarboMer | 03-DS 1550 | W0043-23 | ~1317-1359 |
| L-α-phosphatidylcholine | | Sigma | P-7081 | 055H8377 | 565.7 |
| didecanoyl | | | | | |
| Edetate Disodium | USP | Spectrum | ED150 | TF0419 | 372.2 |
| EDTA disodium magnesium | | Aldrich | 317810 | 05618TB | 358.51 |
| salt | | | | | |
| EDTA disodium zinc salt | | Riedel-de | 34553 | 22820 | 471.63 |
| | | Haen | | | |
| Benzalkonium Chloride | NF | Spectrum | B1068 | SH0391 | ~350 |
| Sorbitol | NF | Spectrum | SO219 | 1122N-01039-1 | 182.2 |
| α-Lactose monohydrate | NF | Spectrum | LA106 | 1335N-00985-1 | 360 |
| Sodium Chloride | USP | Sigma | | 1008Q-01056-1 | 58.44 |
| 9% Triton X-100 | | Promega | G182A | 17491001 | |
| Sterile water for irrigation | USP | Spectrum | S1944 | J4H196 | |

Table 2 illustrates the source and components of the MatTek EpiAirway™ System that is described in greater detail in Example 2 of the instant application.

TABLE 2
In Vitro Epithelial Cell Model System Components

| Reagent | Vendor | Cat # | Lot # |
|---|---|---|---|
| Tissue Culture Inserts | MatTek Corp. | AIR-100 | |
| Serum Free Media | MatTek Corp. | AIR-100-MM | 112604RJJ PRF |

Table 3 illustrates the source and components of the LDH assay system described in greater details in Example 2 of the instant application.

TABLE 3
LDH (Cytotoxicity) and MTT (Cell Viability) Assay Components.

| Reagent | Vendor | Part # | Lot # |
|---|---|---|---|
| Substrate Mix (LDH assay) | Promega | G179A | 18523201 |
| Assay Buffer (LDH assay) | Promega | G180A | 18366001 |
| Stop Solution (LDH assay) | Promega | G183A | 18585401 |
| MTT concentrate (MTT assay) | MatTek | MTT-100-CON | 111604tta |
| | Corp. | | |
| MTT diluent (MTT assay) | MatTek | MTT-100-CON | 112904HC |
| | Corp. | | |
| Extractant Solution (MTT assay) | MatTek | MTT-100-CON | 438B15 |
| | Corp. | | |

Table 4 illustrates the instruments and other related laboratory supplies and source of each used herein.

TABLE 4
Instruments and Other Related Supplies

| Instrument | Vendor | Model # | s/n |
|---|---|---|---|
| Tissue Resistance | World Precision | ENDOHM-12 | 67107 H11D |
| Measurement Chamber | Instruments | | |
| Epithelial | World Precision | EVOM | 60916 G08C |
| Voltohmeter | Instruments | | |
| μQuant optical | Biotek | | 160155 |
| density plate | Instruments | | |
| reader | | | |
| Advanced Micro | Advanced | 2020 | P04030199A |
| Osmometer | Instruments Inc. | | |
| Millicell-CM blank | Millipore | PICM01250 | F2NN64661 |
| insert | | | |
| 24 well Multiwell | Becton Dickinson | 35-3047 | |
| plate | | | |
| 6 well Multiwell | Becton Dickinson | 35-3046 | |
| plate | | | |

### Example 2

### In Vitro Permeation Kinetics of Glucagon-Like Peptide-1 (GLP-1) Pharmaceutical Formulations

The present example demonstrates the exemplary pharmaceutical formulations of the present invention, which contain the excepients DDPC, EDTA and M-β-CD alone or in combination, enhance GLP-1 permeation across an epithelial cell monolayer. Table 5 illustrates the formulations screened in the in vitro EpiAirway Model System by transepithelial resistance assay (TEER), cell viability assay (MTT), lactate dehydrogenase cell death assay (LDH) and tissue permeation assay to determine which formulation achieved the greatest degree of GLP-1 tissue permeation and TEER reduction while resulting in no significant cell toxicity.

Triplicate samples of each formulation and controls were evaluated using the EpiAirway System in vitro model tracheal/bronchial epithelial cell membrane inserts by MatTek Corp. (Ashland, Mass.), catalog #Air-100. The EpiAirway™ system was developed by MatTek Corp (Ashland, Mass.) as a model of the pseudostratified epithelium lining the respiratory tract. The epithelial cells are grown on porous membrane-bottomed cell culture inserts at an air-liquid interface, which results in differentiation of the cells to a highly polarized morphology. The apical surface is ciliated with a microvillous ultrastructure and the epithelium produces mucus (the presence of mucin has been confirmed by immunoblotting). The inserts have a diameter of 0.875 cm, providing a surface area of 0.6 cm². The cells are plated onto the inserts at the factory approximately three weeks before shipping.

EpiAirway™ culture membranes were received the day before the experiments started. They were shipped in phenol red-free and hydrocortisone-free Dulbecco's Modified Eagle's Medium (DMEM). Each tissue insert was placed into a well of a 6 well plate containing 0.9 ml of serum free DMEM. The membranes were then cultured for 24 hrs at 37° C./5% CO₂ to allow tissues to equilibrate. This DMEM-based medium is serum free but is supplemented with epidermal growth factor and other factors. The medium is always tested for endogenous levels of any cytokine or growth factor which is being considered for intranasal delivery, but has been free of all cytokines and factors studied to date except insulin. The volume is sufficient to provide contact to the bottoms of the units on their stands, but the apical surface of the epithelium is allowed to remain in direct contact with air. Sterile tweezers are used in this step and in all subsequent steps involving transfer of units to liquid-containing wells to ensure that no air was trapped between the bottoms of the units and the medium.

The EpiAirway™ model system was used to evaluate the effect of each GLP-1 containing formulation on TEER, cell viability (MTT assay), cytotoxicity (LDH assay) and permeation. These assays are described below in detail.

### Transepithelial Electrical Resistance (TEER)

TEER measurements were read using a Tissue Resistance Measurement Chamber connected to an Epithelial Voltohmeter with the electrode leads, both from World Precision Instruments. First, background TEER was read for each insert on the day the experiment began. After TEER was read, 1 ml fresh media was placed in the bottom of each well in a 6-well plate. Inserts were drained on paper towel and placed into the new wells with fresh media, while keeping the inserts numbered to correlate with background TEER measurements. 100 ul of experimental formulation was added to each insert. Inserts were placed in a shaking incubator at 100 rpm and 37° C. for 1 hr.

The electrodes and a tissue culture blank insert were equilibrated for at least 20 min in fresh media with the power off prior to checking calibration. The background resistance was measured with 1.5 ml media in the Endohm tissue chamber and 300 μl media in a blank Millicell-CM insert. The top electrode was adjusted so that it was submerged in the media but not making contact with the top surface of the insert membrane. Background resistance of the blank insert was 5-20 ohms. For each TEER determination, 300 μl media was added to the insert followed by a 20 min incubation at RT before placement in the Endohm chamber to read TEER. Resistance was expressed as (resistance measured−blank)×0.6 cm². All TEER values were reported as a function of the surface area of the tissue.

TEER was calculated as:

TEER=(R_{I}−R_{b})×A

Where R_{I} is resistance of the insert with a membrane, R_{b} is the resistance of the blank insert, and

A is the area of the membrane (0.6 cm²). A decrease in TEER value relative to the control value (control=approximately 1000 ohms-cm²; normalized to 100.) indicates a decrease in cell membrane resistance and an increase in mucosal epithelial cell permeability. After the 1 hr incubation was complete, the tissue inserts were removed from the incubator. 200 μl fresh media was placed in each well of a 24-well plate and tissue inserts were transferred to the 24-well plate. 200 μl fresh media was gently added to each tissue insert. TEER was again measured for each insert.

After the tissue culture inserts were transferred from the 6-well plate to the 2-well plate, the basal media was subdivided into three parts and stored in eppendorf tubes. All three subdivions were placed at −80° C. until use.

### Lactate Dehydrolenase (LDH) Assay

The amount of cell death was assayed by measuring the release of LDH from the cells using a CytoTox 96 Cytotoxicity Assay Kit, from Promega Corp. Triplicate samples were performed for each tissue culture insert in the study. 50 μl harvested media (stored at 4° C.) was loaded in triplicate in a 96-well plate. Fresh, cell-free media was used as a blank. 50 μl substrate solution (12 ml Assay Buffer added to a fresh bottle of Substrate Mix, made according to the kit) was added to each well and the plates were incubates for 30 min at RT in the dark. Following incubation, 50 μl of stop solution was added to each well and the plates were read on a μQuant optical density plate reader at 490 nm using KCJr software.

### MTT Assay

The cell viability of each tissue culture insert was tested by MTT assay. Cell viability was assessed using the MTT assay (MTT-100, MatTek kit). This kit measures the uptake and transformation of tetrazolium salt to formazan dye. MTT concentrate was thawed and diluted with media at a ratio of 2 ml MTT:8 ml media. The diluted MTT concentrate was pipetted (300 μl) into a 24-well plate. Tissue inserts were gently dried, placed into the plate wells, and incubated for three hours in the dark at 37° C. After incubation, each insert was removed from the plate, blotted gently, and placed into a 24-well extraction plate. The cell culture inserts were then immersed in 2.0 ml of the extractant solution per well (to completely cover the sample). The extraction plate was covered and sealed to reduce evaporation of extractant. After an overnight incubation at room temperature in the dark, the liquid within each insert was decanted back into the well from which it was taken, and the inserts discarded. The extractant solution (50 μl) from each well was pipetted in triplicate into a 96-well microtiter plate, along with extract blanks and diluted with the addition of 150 μl of fresh extractant solution. The optical density of the samples was measured at 550 nm on a μQuant optical density plate reader using KCJr software.

### Tissue Permeation Assay

The quantity of GLP-1 (7-36) that passed from the apical surface to the basolateral surface of the EpiAirway™ epithelial cell monolayer represented the degree of GLP-1 permeation. The quantity of GLP-1 protein found on the basolateral surface of the cultured cells was measured by ELISA. The GLP-1 (7-36) amide ELISA kit was purchased from Linco Research, (St. Charles, Mich.). The ELISA assay was performed in accordance with the manufacturer's protocol. The collected samples were diluted with assay buffer provided with the kit. Several rounds of in vitro screening were performed. Percent permeation was calculated by dividing the measured amount of GLP-1 found on the basolateral side of the cells as measured by ELISA by the total amount of GLP-1 starting material that was added to the apical side of the cells multiplied by 100. First, a design of experiment (DOE) study was performed using different amounts of exciepients EDTA, M-β-CD, and DDPC. Summary of results are in the Table 5. All formulations containing one or more exciepient showed improvement in permeation over the control without exciepients (#31).

TABLE 5
GLP-1 in vitro Permeation Study

| | | | | | Citrate | | | | | | | |
|---|---|---|---|---|---|---|---|---|---|---|---|---|
| | GLP-1 | DDPC | EDTA | M-β-CD | Buffer | Lactose | Sorbitol | NaCl | TEER | MTT | LDH | Permeation |
| # | (mg/mL) | (mg/mL) | (mg/mL) | (mg/mL) | (mM) | (mM) | (mM) | (mM) | (ohms*cm2) | (%) | (%) | (%) |
| 1 | 5 | 0.1 | 1 | 0 | 10 | 25 | 100 | 0 | 16 | 111 | 6 | 2.8 |
| 2 | 5 | 0.5 | 1 | 0 | 10 | 25 | 100 | 0 | 7 | 116 | 5 | 2.7 |
| 3 | 5 | 1 | 1 | 0 | 10 | 25 | 100 | 0 | 7 | 108 | 5 | 1.3 |
| 4 | 5 | 0.1 | 2.5 | 0 | 10 | 25 | 100 | 0 | 6 | 122 | 7 | 2.0 |
| 5 | 5 | 0.5 | 2.5 | 0 | 10 | 25 | 100 | 0 | 5 | 119 | 7 | 1.4 |
| 6 | 5 | 1 | 2.5 | 0 | 10 | 25 | 100 | 0 | 3 | 108 | 6 | 1.6 |
| 7 | 5 | 0.1 | 5 | 0 | 10 | 25 | 100 | 0 | 3 | 128 | 6 | 2.5 |
| 8 | 5 | 0.5 | 5 | 0 | 10 | 25 | 100 | 0 | 3 | 126 | 7 | 2.6 |
| 9 | 5 | 1 | 5 | 0 | 10 | 25 | 100 | 0 | 2 | 111 | 6 | 1.4 |
| 10 | 5 | 0.1 | 1 | 22.5 | 10 | 25 | 100 | 0 | 14 | 119 | 15 | 3.2 |
| 11 | 5 | 0.5 | 1 | 22.5 | 10 | 25 | 100 | 0 | 7 | 97 | 25 | 10.3 |
| 12 | 5 | 1 | 1 | 22.5 | 10 | 25 | 100 | 0 | 31 | 94 | 11 | 2.5 |
| 13 | 5 | 0.1 | 2.5 | 22.5 | 10 | 25 | 100 | 0 | 6 | 117 | 15 | 8.7 |
| 14 | 5 | 0.5 | 2.5 | 22.5 | 10 | 25 | 100 | 0 | 4 | 95 | 23 | 6.9 |
| 15 | 5 | 1 | 2.5 | 22.5 | 10 | 25 | 100 | 0 | 3 | 96 | 30 | 8.3 |
| 16 | 5 | 0.1 | 5 | 22.5 | 10 | 25 | 100 | 0 | 4 | 111 | 8 | 5.6 |
| 17 | 5 | 0.5 | 5 | 22.5 | 10 | 25 | 100 | 0 | 3 | 99 | 11 | 8.3 |
| 18 | 5 | 1 | 5 | 22.5 | 10 | 25 | 100 | 0 | 7 | 79 | 20 | 7.9 |
| 19 | 5 | 0.1 | 1 | 45 | 10 | 25 | 100 | 0 | 17 | 96 | 19 | 4.3 |
| 20 | 5 | 0.5 | 1 | 45 | 10 | 25 | 100 | 0 | 15 | 88 | 13 | 6.8 |
| 21 | 5 | 1 | 1 | 45 | 10 | 25 | 100 | 0 | 7 | 77 | 36 | 8.9 |
| 22 | 5 | 0.1 | 2.5 | 45 | 10 | 25 | 100 | 0 | 3 | 99 | 29 | 10.9 |
| 23 | 5 | 0.5 | 2.5 | 45 | 10 | 25 | 100 | 0 | 4 | 108 | 24 | 8.9 |
| 24 | 5 | 1 | 2.5 | 45 | 10 | 25 | 100 | 0 | 3 | 92 | 29 | 9.4 |
| 25 | 5 | 0.1 | 5 | 45 | 10 | 25 | 100 | 0 | 3 | 104 | 27 | 8.2 |
| 26 | 5 | 0.5 | 5 | 45 | 10 | 25 | 100 | 0 | 2 | 85 | 37 | 8.1 |
| 27 | 5 | 1 | 5 | 45 | 10 | 25 | 100 | 0 | 3 | 74 | 54 | 8.4 |
| 28 | 5 | 0 | 5 | 0 | 10 | 0 | 0 | 0 | 2 | 126 | 10 | 5.0 |
| 29 | 5 | 0 | 10 | 0 | 10 | 0 | 0 | 0 | 33 | 114 | 10 | 6.5 |
| 30 | 5 | 0 | 0 | 0 | 10 | 25 | 100 | 0 | 106 | 116 | 4 | 0.6 |
| 31 | 5 | 0 | 0 | 0 | 10 | 0 | 0 | 140 | 143 | 105 | 2 | 0.07 |

The intranasal pharmaceutical formulations tested in further in vitro TEER, MTT, LDH and % permeation studies are shown in Table 6. The TEER, MTT, LDH and permeation results for these formulations are summarized in Table 7.

TABLE 6
GLP-1 Formulations Tested in Vitro

| | | | | | | Citrate | | | | |
|---|---|---|---|---|---|---|---|---|---|---|
| | GLP-1 | DDPC | EDTA | Mg EDTA | MβCD | Buffer | Lactose | Sorbitol | NaCl | |
| Sample # | (mg/mL) | (mg/mL) | (mg/mL) | (mg/mL) | (mg/mL) | (mM) | (mM) | (mM) | (mg/mL) | pH |
| 1 | 2 | 1 | 1 | 0 | 45 | 10 | 25 | 100 | 0 | 3.5 |
| 2 | 2 | 1 | 1 | 0 | 45 | 10 | 0 | 0 | 0 | 3.5 |
| 3 | 2 | 0 | 0 | 0 | 45 | 10 | 0 | 0 | 0 | 3.5 |
| 4 | 2 | 1 | 0 | 0 | 45 | 10 | 0 | 0 | 0 | 3.5 |
| 5 | 2 | 0 | 1 | 0 | 0 | 10 | 0 | 0 | 0 | 3.5 |
| 6 | 2 | 0 | 10 | 0 | 0 | 10 | 0 | 0 | 0 | 3.5 |
| 7 | 2 | 0 | 0 | 10 | 0 | 10 | 0 | 0 | 0 | 3.5 |
| 8 | 2 | 0 | 0 | 10 | 0 | 10 | 0 | 0 | 0 | 5 |
| 9 | 2 | 0 | 0 | 1 | 0 | 10 | 0 | 0 | 0 | 3.5 |
| 10 | 2 | 1 | 0 | 1 | 45 | 10 | 0 | 0 | 0 | 3.5 |
| 11 | 2 | 1 | 0 | 10 | 45 | 10 | 0 | 0 | 0 | 3.5 |
| 12 | 2 | 1 | 10 | 0 | 45 | 10 | 0 | 0 | 0 | 3.5 |
| 13 | 2 | 0 | 0 | 0 | 0 | 10 | 0 | 0 | 140 | 3.5 |
| Media | 0 | 0 | 0 | 0 | 0 | 0 | 0 | 0 | 0 | n/a |
| LDH | 0 | 0 | 0 | 0 | 0 | 0 | 0 | 0 | 0 | n/a |
| Control | | | | | | | | | | |

TABLE 7
Summary of GLP-1 in Vitro Results

| | | | | | | Permeation | | |
|---|---|---|---|---|---|---|---|---|
| | | | | | | Fold | | |
| | | | | | | increase | | |
| Sample | Actually | | % TEER | % | % | over | % | % |
| # | pH | Appearance (T = 0) | Reduction | MTT | LDH | control | Avg | Stdev |
| 1 | 3.45 | Clear and Colorless | 98 | 86 | 20 | 202 | 4.9 | 0.6 |
| 2 | 3.49 | Clear and Colorless | 98 | 85 | 19 | 310 | 7.4 | 0.3 |
| 3 | 3.42 | Clear and Colorless | 95 | 100 | 8 | 86 | 2.1 | 0.4 |
| 4 | 3.45 | Clear and Colorless | 95 | 90 | 9 | 74 | 1.8 | 0.3 |
| 5 | 3.41 | Clear and Colorless | 98 | 120 | 8 | 112 | 2.7 | 0.2 |
| 6 | 3.75 | Clear and Colorless | 100 | 96 | 9 | 256 | 6.1 | 0.3 |
| 9 | 3.75 | Clear and Colorless | 96 | 115 | 10 | 88 | 2.1 | 0.6 |
| 10 | 3.79 | Clear and Colorless | 97 | 82 | 13 | 161 | 3.9 | 1.3 |
| 11 | 3.42 | Clear and Colorless | 99 | 88 | 11 | 89 | 2.1 | 0.7 |
| 12 | 3.78 | Clear and Colorless | 99 | 53 | 27 | 341 | 8.2 | 1.0 |
| 13 | 3.60 | Clear and Colorless | 37 | 109 | 6 | 1 | 0.0 | 0.0 |
| Media | n/a | n/a | −35 | 100 | 6 | n/a | n/a | n/a |
| LDH | n/a | n/a | 100 | 1 | 100 | n/a | n/a | n/a |
| Control | | | | | | | | |

A measured decrease in TEER value relative to the control indicates a decrease in cell membrane resistance or in other words the passage of ionic species from the apical to the basolateral side of the epithelial monolayer. The data presented in Table 7 indicates that all enhancer formulations significantly reduced TEER compared to the control formulations.

The MTT assay measured cell viability while the LDH assay measured cytotoxicity. The assays are used in combination in order to determine the effect of pharmaceutical formations on cell “health.” The MTT and LDH results were both expressed as a percent. The MTT percentage was calculated by dividing the measured MTT value for each formulation by the MTT value of the control formulation multiplied by 100. Thus, the MTT positive control was 100% and served as the base-line comparison for all other formulations. A MTT value below 80% represents a negative effect on cell viability. Similarly, the LDH percentage was calculated by dividing the measured LDH value for each formulation by the LDH value of the control formulation multiplied by 100. Thus, the LDH positive control was 100% and served as the base-line comparison for all other formulations. The results of the MTT assay indicate that all but one formulation (#12 in Table 7) did not reduce cell viability below 80%. This data was further supported by the LDH cytotoxicity assay that showed that a majority of the formulations did not show significant levels of cytotoxicity.

GLP-1 tissue permeation is expressed as % permeation and fold-increase over that of the control formulation (sample #13). The fold increase over the control for the excipient containing formulations enhanced GLP-1 permeation from approximately 74-fold to 341-fold over that of the control. These data indicate the inclusion of the excipients DDPC, EDTA and M-β-CD significantly enhance GLP-1 permeation across an epithelial cell monolayer. From Table 7, formulations #1, #2, #6, and #12 resulted in >200 fold improvement of % permeation over control without excipient (#13 in Table 7).

In summary, the in vitro data indicate that the exemplary pharmaceutical formulation of the present invention, comprising 2 mg/ml GLP-1, 10 mg/ml EDTA and 10 mM Citrate Buffer (sample #6 in Table 6), exhibited the greatest GLP-1 permeation enhancing and TEER reducing qualities while having the a minimal negative effect on cell viability. Thus, this formulation represents an ideal candidate for the delivery of GLP-1 across a mucosal surface, for example intranasal (IN) drug delivery, in the treatment of human disease including obesity and diabetes.

### Example 3

### In Vitro Permeation Kinetics Comparison of Glucagon-Like Peptide-1 (GLP-1) Pharmaceutical Formulations Containing EDTA, EDTA Zinc Salts or EDTA Magnesium Salts

The present example demonstrates that in vitro permeation kinetics of GLP-1 pharmaceutical formulations are sensitive to the form of EDTA used in the formulation. Table 8 below illustrates the formulations screened in the in vitro EpiAirway Model System by transepithelial resistance (TEER assay), cell viability (MTT assay), lactate dehydrogenase (LDH assay; cell death) and tissue permeation. All samples contained 2 mg/ml GLP-1 except samples #9 and #10 which served as controls. Formulations were used within 24 hours of manufacture and therefore no preservatives were added. Each formulation was made to a total volume of 0.5 ml and evaluated in triplicate (n=3). Each sample was evaluated according the protocols described in detail above in Example 2.

TABLE 8
Formulations Containing Different Forms of EDTA
Screened for GLP-1 Permeation Enhancement.

| Sample | Composition | Comments |
|---|---|---|
| 1 (n = 3) | 2 mg/mL GLP-1 (0.6 mM), 45 mg/mL M-β- | |
| | CD, 1 mg/mL EDTA, 1 mg/mL DDPC, 10 | |
| | mM citrate (pH 3.5), 25 mM lactose, 100 mM | |
| | sorbitol | |
| 2 (n = 3) | 2 mg/mL GLP-1, 10 mg/mL EDTA, 10 mM | 10 mg/mL EDTA as |
| | citrate buffer (pH 3.5) | enhancer and hypotonic |
| | | (~50 mOsm/kg) |
| 3 (n = 3) | 2 mg/mL GLP-1, 10 mg/mL EDTA, 0.1 | Same as #2 with 0.1 |
| | mg/ml DDPC, 10 mM citrate buffer (pH 3.5) | mg/mL DDPC as |
| | | additional enhancer |
| 4 (n = 3) | 2 mg/mL GLP-1, 10 mg/mL Zn EDTA, 10 | Same as #2 but with Zn |
| | mM citrate buffer (pH 3.5) | EDTA |
| 5 (n = 3) | 2 mg/mL GLP-1, 10 mg/mL Mg EDTA, 10 | Same as #2 but with Mg |
| | mM citrate buffer (pH 3.5) | EDTA |
| 6 (n = 3) | 2 mg/mL GLP-1 (0.6 mM), 45 mg/mL M-β- | Same as #1 but with Zn |
| | CD, 1 mg/mL Zn EDTA, 1 mg/mL DDPC, 10 | EDTA |
| | mM citrate (pH 3.5), 25 mM lactose, 100 mM | |
| | sorbitol | |
| 7 (n = 3) | 2 mg/mL GLP-1 (0.6 mM), 45 mg/mL M-β- | Same as #1 but with Mg |
| | CD, 1 mg/mL Mg EDTA, 1 mg/mL DDPC, | EDTA |
| | 10 mM citrate (pH 3.5), 25 mM lactose, 100 | |
| | mM sorbitol | |
| 8 (n = 3) | 2 mg/mL GLP-1, 10 mM citrate (pH 3.5), 150 | GLP-1 Negative Control |
| | mM NaCL | (no enhancers) |
| 9 (n = 3) | MatTek Media | MTT positive control; LDH |
| | | negative control; TEER |
| | | negative control |

| | |
|---|---|
| 10 (n = 3) | 9% Octylphenolpoly(ethyleneglycolether)x (TritonX-100 ™) |
| | (LDH positive control; TEER positive control; MTT negative control) |

Abbreviations:
M-β-CD = methyl-beta-cyclodextrin, EDTA = disodium edetate, DDPC = L-α- phosphatidylcholine didecanoyl, CB = chlorobutanol, Mg EDTA = EDTA disodium magnesium salt; Zn EDTA = EDTA disodium zinc salt; MTT = MTT assay; LDH = LDH assay; TEER = transepithelial resistance.

### Results

The effect of pharmaceutical formulations comprising intranasal delivery-enhancing agents, for example, the excipients DDPC, EDTA and M-β-CD on an EpiAirway™ Cell Membrane (mucosal epithelial cell layer) is shown. The permeation kinetics results are summarized below in Table 9 and represent measurements taken one hour after cells were incubated with the formulations shown in Table 8.

TABLE 9
Permeation Kinetic Results for GLP-1 Formulations
Containing Different EDTA Salts

| | | | | | Permeation |
|---|---|---|---|---|---|
| Sample | % TEER | MTT | LDH | Permeation | Fold Increase |
| # | Reduction | (%) | (%) | (%) | Over Control |
| 1 | 3 | 89 | 3 | 5.3 | 83 |
| 2 | 1 | 123 | 1 | 3.2 | 50 |
| 3 | 1 | 107 | 3 | 2.6 | 41 |
| 4 | 11 | 109 | 3 | 1.0 | 16 |
| 5 | 1 | 123 | 4 | 5.5 | 88 |
| 6 | 7 | 85 | 8 | 2.0 | 31 |
| 7 | 5 | 100 | 9 | 4 | 63 |
| 8 | 28 | 109 | 3 | 0.1 | 1 |
| 9 | 109 | 88 | 1 | N/A | N/A |
| 10 | 0 | 1 | 100 | N/A | N/A |

The foregoing data indicate that various EDTA salt forms can be used in pharmaceutical formulations to manipulate drug permeation kinetics.

### Example 4

### GLP-1 Stability

The present example demonstrates that small molecule excipients, for example M-β-CD, EDTA and DDPC, do not promote GLP-1 physical stability in pharmaceutical formulations. In the instant example, GLP-1 stability was evaluated with the two formulations described below in Table 10. The purpose of the instant example was to determine whether heating GLP-1 causes protein degradation.

TABLE 10
GLP-1 Stability Formulations

| Sample | Composition | Testing |
|---|---|---|
| 1 (50 mL) | 45 mg/mL M-β-CD, 1 mg/mL EDTA, 1 | pH, |
| | mg/mL DDPC, 10 mM citrate (pH 3.5), | Appearance, |
| | 25 mM lactose, 100 mM sorbitol | DSC |
| 2 (50 mL) | 10 mM citrate (pH 3.5) | pH, |
| | | Appearance, |
| | | DSC |

One differential scanning caliorimetry (DSC) experiment with a rescan was performed on Sample #1 of Table 10; the sample was scanned from 5° C. to 100° C. at a scan rate of 60° C./hour. The data was graphed as Cp (ca/° C.) v. temperature (° C.). A sharp transition peak is observed near 35° C. in the first heat scan. The noise following the transition and the large decrease in heat capacity between 80 and 90° C. suggest the formation of aggregates. The solution was observed to be slightly cloudy following the scans, supporting the formation of a precipitate. The transition peak is much narrower than expected for unfolding of a peptide, this may be the result of aggregation immediately following unfolding of the peptide. These data indicate that the GLP-1 begins to denature and/or forms aggregates in formulation at or around 35° C.

A DSC experiment with a rescan was performed on Sample #2; the sample was scanned from 5 to 100° C. at a scan rate of 60° C./hour. The data was graphed as Cp(cal/° C.) vs. temperature (° C.). A very broad peak was observed near 46° C. in the first heat scan. The large decrease in heat capacity between 80 and 90° C. may be due to the formation of aggregates. However, the solution did not appear cloudy following the scans suggesting very small aggregates. These data indicate that the GLP-1 begins to denature and/or forms aggregates in formulation at or around 46° C.

These data indicate that the addition of excipients as described in Table 10 above to the GLP-1 formulation does not enhance its physical stability.

### Example 5

### Pharmacokinetic (PK) Evaluation of Intranasal and Intravenous Administration of Glucagon-Like Peptide-1 (GLP-1) in Selected Pharmaceutical Formulations in Rabbits

The present example demonstrates that GLP-1 bioavailability by intranasal administration is significantly enhanced by the inclusion of a dipeptidyl peptidase-IV (DPP-IV) inhibitor, for example Lys(4-nitro-Z)-pyrrolidide, in the pharmaceutical formulation.

A pharmacokinetic (PK) study in rabbits was performed to evaluate the plasma pharmacokinetic properties of GLP-1 with various formulations administered via intranasal (IN) delivery versus intravenous (IV) infusion. The overall study design is presented below in Table 11. Formulations #1 through #4 represent the IN formulations while formulation #5 represents the IV infused formulation.

TABLE 11
Overall Study Design for the Pharmacokinetic Evalution

| | | Route of | GLP-1 | Dose | GLP-1 |
|---|---|---|---|---|---|
| Study | | Administration | Conc. | Volume | Dose Level |
| Groups | Animals | (Formulation) | (mg/mL) | (mL/kg) | (μg/kg) |
| #1 | 5M | Intranasal | 5 | 0.015 | 75 |
| | | (Formulation 1) | | | |
| #2 | 5M | Intranasal | 5 | 0.015 | 75 |
| | | (Formulation 2) | | | |
| #3 | 5M | Intranasal | 5 | 0.015 | 75 |
| | | (Formulation 3) | | | |
| #4 | 5M | Intranasal | 5 | 0.015 | 75 |
| | | (Formulation 4) | | | |
| #5 | 5M | Intravenous | 0.075 | 0.1 | 7.5 |
| | | (Formulation 5) | | | |

In this study, New Zealand White rabbits (Hra: (NZW) SPF) were used as test subjects to evaluate plasma pharmacokinetics of GLP-1 by intranasal administration and intravenous infusion. Rabbits were chosen as animal subjects for this study because the pharmacokinetic profile derived from a drug administered to rabbits closely resembles the PK profile for the same drug in humans.

Four intranasal formulations and one intravenous formulation of GLP-1 were evaluated in the study. The vehicle composition for each formulation is provided in Table 12. The GLP-1 intranasal and intravenous formulations were manufactured for final preparation and testing.

For each of the dosing solutions, the components were provided in two parts, Part A and Part B (see Table 12). The final formulation for each of the intranasal groups (Groups 1-4) was created by mixing equal volumes of Part A (1 mL) and Part B (1 mL). For the intravenous group (Group 5), 1.5 mL of Part A and 3.5 mL Part B were mixed. Final dosing solutions for all groups were used within 6 hours of preparation.

TABLE 12
Vehicle Composition for GLP-1 Formulations 1 Through 5

| Com- | | Concentration | | | | |
|---|---|---|---|---|---|---|
| ponent | | Part A | | Part B | Final | |
| Formu- | GLP-1 | 10 | mg/mL | N/A | 5 | mg/mL |
| lation | Citrate | 20 | mM | N/A | 10 | mM |
| 1 | EDTA | 20 | mg/mL | N/A | 10 | mg/mL |
| | Lys(4-nitro-Z)- | N/A | | 50 mM | 25 | mM |
| | pyrrolidide | | | | | |
| | pH = 3.5 | | | | | |
| Formu- | GLP-1 | 10 | mg/mL | N/A | 5 | mg/mL |
| lation | Citrate | 20 | mM | N/A | 10 | mM |
| 2 | PN159 | 100 | μM | N/A | 50 | μM |
| | Lys(4-nitro-Z)- | N/A | | 50 mM | 25 | mM |
| | pyrrolidide | | | | | |
| | pH = 3.5 | | | | | |
| Formu- | GLP-1 | 10 | mg/mL | N/A | 5 | mg/mL |
| lation | Citrate | 20 | mM | N/A | 10 | mM |
| 3 | EDTA | 20 | mg/mL | N/A | 10 | mg/mL |
| | Lys(4-nitro-Z)- | N/A | | 30 mM | 15 | mM |
| | pyrrolidide | | | | | |
| | pH = 3.5 | | | | | |
| Formu- | GLP-1 | 10 | mg/mL | N/A | 5 | mg/mL |
| lation | Citrate | 20 | mM | N/A | 10 | mM |
| 4 | EDTA | 20 | mg/mL | N/A | 10 | mg/mL |
| | pH = 3.5 | | | | | |
| Formu- | GLP-1 | 0.25 | mg/mL | N/A | 0.075 | mg/mL |
| lation | Citrate | 2 | mM | N/A | 0.6 | mM |
| 5 | EDTA | 2 | mg/mL | N/A | 0.6 | mg/mL |
| | Sodium Chloride | 300 | mM | N/A | 90 | mM |

| | | | | |
|---|---|---|---|---|
| Lys(4-nitro-Z)- | N/A | 5 mM | 3.5 | mM |
| pyrrolidide | | | | |

pH = 3.5

The concentration of GLP-1 was constant among the four intranasal formulations. The citrate concentration and pH were also consistent among the four formulations. The EDTA concentration was consistent for formulations 1, 3, and 4. Formulation 2 contained PN159, a tight junction modulator previously shown to enhance the delivery of peptides across an epithelial cell layer, but no EDTA. Formulation 5 contained citrate, EDTA, and sodium chloride, each at the appropriate concentration for intravenous administration; whereas the GLP-1 concentration was decreased to provide a GLP-1 total dose that was 10% of the intranasal dose.

Lys(4-nitro-Z)-pyrrolidide is a specific inhibitor of dipeptidyl aminopeptidase (DPP) IV, the primary enzyme responsible for the metabolism of active GLP-1 (7-36 amino acid fragment) to an inactive metabolite (9-36 amino acid fragment). For evaluation in this study, the concentrations of Lys(4-nitro-Z)-pyrrolidide was varied for each of the intranasal dose groups with the exception of equal concentration between Formulation 1 and Formulation 2. The total dose of Lys(4-nitro-Z)-pyrrolidide was approximately 0.04 mMoles/kg for groups 1 and 2 (intranasal groups), and group 5 (intravenous group).

### GLP-1 Assay Method

Study samples, standards, and quality control samples were assayed with a Glucagon Like Peptide-1 (Active) ELISA Kit (Linco Research Inc. Catalog #EGLP-35K). Each sample was analyzed in duplicate.

This assay is based on the capture of active GLP-1 (7-36 and 7-36amide fragments) by a monoclonal antibody (specific to the N-terminal region) immobilized in the wells of a 96-well microtiter plate, and detection by a second anti-GLP-1 alkaline phosphatase-labeled antibody. After washing, methyl umbelliferyl phosphate is added to each well, which in the presence of alkaline phosphatase forms the fluorescent product umbelliferone. The amount of fluorescence generated is directly proportional to the concentration of active GLP-1 in an unknown sample, and this was derived by interpolation from a reference curve using reference standards of known concentration of active GLP-1.

Due to species similarity between human and rabbit GLP-1, it was anticipated the assay would detect endogenous (i.e., rabbit) active GLP-1. Endongenous levels of rabbit GLP-1 are measured at time 0. The 9-36 GLP-1 fragment was not detected by the assay, regardless of source.

### Pharmacokinetic Evaluation

Pharmacokinetic calculations were performed using WinNonlin software (Pharsight Corporation, Version 4.0, Mountain View, Calif.) and a non-compartmental model of extravascular administration. The parameters for evaluation are described in Table 13.

TABLE 13
Pharmacokinetic Parameters

| | |
|---|---|
| Kel | Apparent terminal phase rate constant, where Kel is the |
| | magnitude of the slope of the linear regression of the log |
| | concentration versus time profile during the terminal phase. |
| t_{1/2} | Apparent terminal phase half-life (whenever possible), |
| | where t_{1/2} = (In2)/Kel |
| Tₘₐₓ | Time to maximum observed concentration of drug in |
| | subject's blood |
| Cₘₐₓ | Maximum observed concentration of drug in subject's |
| | blood |
| Tₘₐₓ | Time to maximum observed concentration of drug in |
| | subject's blood |
| AUCₗₐₛₜ | Area under the concentration-time curve from time 0 |
| | (prior to dosing) to time t, calculated by the linear |
| | trapezoidal rule, where t is the time point of the |
| | last measurable concentration. |
| CL/CL_F | Clearance. CL = Dose/AUC. For extravascular models the |
| | fraction of dose absorbed may not be estimated, |
| | therefore Clearance for these models is actually |
| | CL_F where F is the fraction of dose (bioavailability) |
| | absorbed. |

### Pharmacokinetic Parameters Analysis

The mean GLP-1 pharmacokinetic data for both the intranasal and intravenous groups are provided in Table 14.

Pre-dose (baseline) concentrations of endogenous GLP-1 in serum or plasma were generally below 10 pg/mL. For some samples, limitations on sample volume precluded repeated analysis to obtain definitive results. A value of <4 pg/mL was reported for these samples. To calculate group mean values, and for pharmacokinetic evaluation, data were baseline corrected when appropriate. Results denoted by <NUMBER were set at “<NUMBER/2”; with this approach a value of <4 pg/mL was set at 2 pg/mL.

Post-dosing, individual animal plasma values for GLP-1 exceeded the 10 pg/mL baseline value. At the final time point after intravenous infusion (90 minutes), plasma concentrations of GLP-1 were at or near baseline values indicating the infusion and elimination phases were captured by the sampling time frame employed for the study. Following intranasal instillation, several animals had GLP-1 plasma concentrations that exceeded baseline levels. The AUC_{inf} was determined to estimate the entire exposure profile for GLP-1.

TABLE 14
Mean Pharmacokinetic Parameters for GLP-1 in Plasma of Male Rabbits Following
Intranasal (Groups 1-4) and Intravenous (Group 5) Instillation

| | | | | | | AUClast | AUCInf | Cl_F |
|---|---|---|---|---|---|---|---|---|
| | Dose | Kel | t½ | Tmax | Cmax | (min*pg/ | (min*pg/ | (ml/min/ |
| Group | (μg/kg) | (1/min) | (min) | (min) | (pg/ml) | mL) | mL) | kg) |
| 1 | 75 | 0.0136 | 55.5 | 11.0 | 844.9 | 20792.7 | 32415.9 | 3032.8 |
| 2 | 75 | 0.0110 | 66.8 | 43.0 | 424.0 | 17069.8 | 33324.4 | 2716.0 |
| 3 | 75 | 0.0259 | 39.3 | 25.0 | 283.9 | 9331.5 | 13232.7 | 6977.0 |
| 4 | 75 | 0.0328 | 22.4 | 47.0 | 154.5 | 7054.7 | 5743.4 | 15316.0 |
| 5 | 7.5 | 0.0243 | 30.9 | 6.3 | 3183.6 | 28883.2 | 29149.5 | 259.8 |

Formulation 1/Group 1

Peak concentrations of GLP-1 (Tₘₐₓ) occurred between 5 and 20 minutes (group mean of 11 minutes) after dose administration. The group mean Cₘₐₓ was 844.9 pg/mL. Plasma concentrations remained above baseline at 90 minutes post-dose, indicating elimination was not completed at this time. This is reflected in the mean AUCₗₐₛₜ of 20792.7 min*pg/mL and AUC_{inf} of 32,415.9 min*pg/mL. The mean terminal half-life (t_{1/2}) was estimated to be 55.5 minutes.

Formulation 2/Group 2

Group 2 mean Cₘₐₓ was estimated to be 424.0 pg/mL. The mean Tₘₐₓ was estimated to be 43 minutes; however, there was considerable inter-animal variability for this parameter. Examination of the concentration vs. time profile for these animals indicated an absorption (increasing) and elimination (decreasing) phase. Group 2 mean AUCₗₐₛₜ was 17,069.8 min*pg/mL. A t_{1/2} could not be estimated because of an absence of a clear elimination phase in two animals within the group. However, based on the data collected for the remaining animals in the group, the mean t_{1/2} was 66.8 minutes. Mean AUC_{inf} (three animals in which Kel could be determined) was 33,324.4 min*pg/mL.

Formulation 3/Group 3

Group 3 mean Cₘₐₓ, Tₘₐₓ, t_{1/2}, and AUCₗₐₛₜ were 283.9 pg/mL, 25 minutes, 39.3 minutes, and 9331.5 min*pg/mL, respectively. An acurrate Kel, and thus t_{1/2}, could not be determined because one animal in the group had a higher than expected value at 60 minutes. Thus, this value was not included in the group mean t_{1/2} of 39.3 minutes and group mean AUC_{inf} of 13,232.7 min*pg/mL.

### Formulation 4/Group 4

The mean Cₘₐₓ for Group 4 was estimated to be 154.5 pg/mL. Group mean Tₘₐₓ was 47 minutes, however, two animals, had their highest measured plasma concentrations of GLP-1 at 90 minutes. Without an apparent elimination phase Kel (and t_{1/2}) and AUC_{inf} could not be determined for these animals. The mean t_{1/2} and AUC_{inf} for the other three animals was estimated to be 22.4 minutes and 5734.4 min*pg/mL, respectively.

Formulation 5/Group 5

During the infusion procedure, one animal of the group 5 experienced a mechanical failure with the pump apparatus, which resulted in an additional ˜100 μl being delivered between the 5 and 10 minute time points. This is reflected in the 10-minute concentration value of >5000 pg/mL. As the exact conditions for the infusion could not be determined, the data for this animal was not included in the pharmacokinetic evaluation for group 5.

The mean Cₘₐₓ for the 10-minute intravenous infusion was 3183.6 pg/mL. Three animals had a Tₘₐₓ at 5 minutes and one animal had a Tₘₐₓ at 10 minutes; although concentrations of GLP-1 were generally similar at the 5- and 10-minute time points for all four animals. A terminal t_{1/2} for the group was estimated to be 30.9 minutes. The mean AUCₗₐₛₜ of 28,883.2 min*pg/mL captured the majority of the exposure profile, as the AUC_{inf} was only slightly greater, 29,149.5 min*pg/mL.

The examination of the log concentration vs. time profile between 10 minutes (end of infusion) and 20 minutes suggested a biphasic elimination of GLP-1. The faster α-phase of a biphasic profile is generally associated with extravascular distribution and elimination, whereas the slower β-phase is considered to represent terminal elimination. Calculation of the elimination rate during this time frame indicated an initial t_{1/2} of 2 minutes or less. This initial t_{1/2} would be consistent with achieving an apparent steady state in the latter half of the 10-minute infusion period.

### Bioavailability

GLP-1 bioavailability after intranasal administration was calculated using AUCₗₐₛₜ or AUC_{inf}; these estimates are provided in Table 15. In Groups 1, 3, and 4, the concentration of the inhibitor Lys(4-nitro-Z)-pyrrolidide in the formulation was 0 mM, 15 mM, or 25 mM, respectively. In the absence of the inhibitor, GLP-1 bioavailability was approximately 2% while the addition of 15 mM Lys(4-nitro-Z)-pyrrolidide in the formulation (Group 3) increased GLP-1 bioavailability to approximately 3% to 5%. GLP-1 bioavailability was further increased to approximately 7% to 11% upon addition of 25 mM Lys(4-nitro-Z)-pyrrolidide in the formulation (Group 1).

The polypeptide PN159 has been shown to increase bioavailability of peptides as compared to small molecule excipients. When tested, formulation 2 (Group 2) containing PN159 and 25 mM Lys(4-nitro-Z)-pyrrolidide had an approximate GLP-1 bioavailability of 6 to 11%, which is equivalent to the bioavailability observed for GLP-1 in the presence of 25 mM Lys(4-nitro-Z)-pyrrolidide without PN159. PN159 has the same effect as 10 mM EDTA on IN bioavailability of GLP-1.

TABLE 15
Bioavailability of GLP-1 in Rabbits Administered
75 μg/kg via Intranasal Instillation (7.5 μg/kg
Intravenous Dose for Calculation; Group 5)

| | Group 1 | Group 2 | Group 3 | Group 4 | Group 5 |
|---|---|---|---|---|---|
| AUCₗₐₛₜ | 20792.7 | 17069.8 | 9331.5 | 7054.7 | 28883.2 |
| (min*pg/mL) | | | | | |
| Bioavailability | 7.2% | 5.9% | 3.2% | 2.4% | N/A |
| AUC_{inf} | 32415.9 | 33324.4 | 13232.7 | 5743.4 | 29149.5 |
| (min*pg/mL) | | | | | |
| Bioavailability | 11.1% | 11.4% | 5.4% | 2.0% | N/A |

^{a} Bioavailability = [Dose (Group 5) × AUCₓₓₓ (Group X)}/[Dose (Group X) × AUCₓₓₓ (Group 5)] × 100.

The terminal half-life for GLP-1 was approximately 30 minutes in rabbit and was longer than anticipated based on published reports showing a terminal half-life of 10 minutes or less for rat and human (Parkes, D., et al., “Phamacokinetic Actions of Exendin-4 in the rat: Comparison with Glucagon-like Peptide-1,” Drug Development Research 53:260-267, 2001; Deacon, C., Therapeutic Strategies Based on Glucagon-like Peptide-1, Perspectives in Diabetes 53:2181-2189, 2004. The t_{1/2} for rabbit was consistent among each of the groups and animals, and thus is supported within the study.

Terminal t_{1/2} calculation is dependent upon the portion of the log concentration vs. time curve used for determination of slope (see definition of Kel in Table 13). For the estimate described above, the modeling program was allowed to pick the best fit for the estimation of Kel. Manual selection for curve fitting from 10 minutes (end of infusion) to 90 minutes indicated a terminal half-life in the range of 10 to 12 minutes (data not shown).

Clearance provides a second means of evaluating disposition of a drug after administration. Clearance can be considered as the intrinsic ability of the body or its organs to remove a drug from the blood (Basic Clinical Pharmacokinetics, 2nd ed., Applied Therapeutics, Inc. Vancouver, Wash.). However, in this case, GLP-1 can remain in the blood, but be considered removed for the purposes of evaluating clearance. The reason for this is that GLP-1 exists in two different states: an active state consisting of a peptide fragment represented by amino acids 7-36 and an inactive state, a result of metabolism, consisting of a peptide fragment represented by amino acids 9-36. Upon conversion to the inactive state, GLP-1 is considered effectively eliminated from the body. As the assay used herein to detect GLP-1 was specific to the active form of GLP-1 (7-36), the presence of the inactive form of GLP-1 in the blood did not interfere with evaluating the clearance of GLP-1.

For the intranasal groups, the highest clearance value (CL-F) was noted for Group 4 with a group mean of 15,160.0 mL/min/kg. The mean value for Group 3 was 6977.0 mL/min/kg. Clearance was similar for Groups 2 and 1,2716.0 mL/min/kg and 3032.8 mL/min/kg, respectively. As would be expected, the clearance estimate for each group is inversely proportional to the systemic exposure.

Clearance values for the intranasal groups were also adjusted for the bioavailability of GLP-1. Adjusted clearance for Groups 4 and 3 were 7580 mL/min/kg and 1395 mL/min/kg, respectively. For Groups 1 and 2, (˜11% bioavailability for each) adjusted clearance values were determined to be 275 mL/min/kg and 247 mL/min/kg, respectively. The adjusted clearance for Groups 1 and 3 were similar to the clearance estimate of 259.8 mL/min/kg for the intravenous dose group (Group 5).

The total dose of Lys(4-nitro-Z)-pyrrolidide was approximately 0.004 mMoles/kg for Groups 1, 2, and 5. Blood levels of Lys(4-nitro-Z)-pyrrolidide were not determined in this study, as such the bioavailability following intranasal administration is not known. The presence of Lys(4-nitro-Z)-pyrrolidide in the nasal formulation has the potential to protect active GLP-1 from metabolism within the nasal mucosa, and assuming reasonable bioavailability, within the systemic circulation. Protection from metabolism by nasal mucosa is consistent with a higher Cₘₐₓ for GLP-1 when Lys(4-nitro-Z)-pyrrolidide was present in the formulation. However, as both fragments of GLP-1 are likely to cross into the circulation, an assay for total GLP-1 (7-36 and 9-36 amino acid fragment) would be required to confirm a higher percentage is active GLP-1.

Protection from metabolism within blood is indicated by the similarity in clearance values for GLP-1 among the intravenous group (Group 5) and the intranasal groups with the highest concentration of inhibitor (Groups 1 and 2).

### SUMMARY

These data show the surprising and unexpected discovery that delivery of intranasal pharamaceutical formulations of GLP-1 with the DPP IV inhibitor Lys(4-nitro-Z)-pyrrolidide resulted in increased GLP-1 bioavailability. Further, GLP-1 bioavailability was dependent upon the concentration of inhibitor in the formulation. In the absence of the inhibitor, the bioavailability of GLP-1 was approximately 2%. Inclusion of Lys(4-nitro-Z)-pyrrolidide at 15 mM increased the GLP-1 bioavailability to approximately 5%, and with 25 mM Lys(4-nitro-Z)-pyrrolidide, bioavailability for GLP-1 was approximately 11%. The potential for local (nasal tissue) and systemic inhibition of the metabolism of GLP-1 was indicated by the data obtained.

Formulation with PN159, in addition to 25 mM Lys(4-nitro-Z)-pyrrolidide, was also investigated to evaluate the potential for greater bioavailability with this tight junction modulator. Under the conditions of this study, the effect of PN159 on the bioavailability of GLP-1 was not greater than the effect of EDTA in the formulation.

### Example 6

### GLP-1 Formulations In-Use and As-Sold Stability

“In-use” stability is defined as those studies involving a formulation stored within a vial affixed with an actuator and sprayed according to the appropriate therapeutic regimen (in this case, three times a day (TID)), and placed at specific storage temperatures. Vials with actuators are primed initially, but are not primed between sprays thereafter. Priming is defined as spraying until a full spray is visually apparent, then actuating one more time before dosing. Vials are stored at 30° C./65% relative humidity (RH) at all times and are sprayed within 10 minutes of removal from the chamber for each dosing. The vials are actuated three times or one time daily, depending on the regimen selected for each study. The time between each spray is at least 1 hr and visual/physical observations were noted.

TID/30° C. in-use studies were performed. The formulation used in this study contains 5 mg/mL GLP-1, 10 mg/mL EDTA, 10 mM Citrate Buffer (pH 3.5), and no preservative. Vials were filled, primed and actuated TID, and stored at 30° C./65% RH for 10 days. The in-use recovery and purity of GLP-1 after 7 days TID of spraying are shown in Table 16 and 17. In-use peptide recovery was greater than 95±2.2% for up to 7 days. In-use total peptide purity was 98±2.1% after 10 days TID/30° C./65% RH.

TABLE 16
GLP-1 In-use Recovery After TID/30° C./65% RH

| Days of | Peptide Concentration (ug/mL) | | | | Peptide Recovery (%) | | | |
|---|---|---|---|---|---|---|---|---|
| Incubation | Vial 1 | Vial 2 | Average | STDEV | Vial 1 | Vial 2 | Average | STDEV |
| 1 | 4663.3 | 4655.6 | 4659.4 | 5.4 | 93.3* | 93.1* | 93.2* | 0.1* |
| 2 | 4624.9 | 4616.1 | 4620.5 | 6.2 | 98.3^{†} | 98.1^{†} | 98.2^{†} | 0.1^{†} |
| 3 | 4523.1 | 4485.0 | 4504.0 | 26.9 | 96.1^{†} | 95.3^{†} | 95.7^{†} | 0.6^{†} |
| 4 | 4482.5 | 4361.9 | 4422.2 | 85.2 | 95.3^{†} | 92.7^{†} | 94.0^{†} | 1.8^{†} |
| 5 | 4517.0 | 4302.1 | 4409.6 | 152.0 | 96.0^{†} | 91.4^{†} | 93.7^{†} | 3.2^{†} |
| 6 | 4812.4 | 4542.3 | 4677.3 | 191.0 | 102.3^{†} | 96.5^{†} | 99.4^{†} | 4.1^{†} |
| 7 | 4557.9 | 4410.1 | 4484.0 | 104.5 | 96.9^{†} | 93.7^{†} | 95.3^{†} | 2.2^{†} |
| 8 | 2743.3 | 2471.8 | 2607.6 | 192.0 | 58.3^{†} | 52.5^{†} | 55.4^{†} | 4.1^{†} |
| 9 | 176.1^{‡} | 230.3^{‡} | 203.2 | 38.4 | 3.7^{†} | 4.9^{†} | 4.3^{†} | 0.8^{†} |
| 10 | 1944.6 | 512.7^{‡} | 1228.7 | 1012.4 | 41.3^{†} | 10.9^{†} | 26.1^{†} | 21.5^{†} |

*% of label claim
^{†}% of T = 0
^{‡}Estimated values

TABLE 17
GLP-1 In-use Purity After TID/30° C./65% RH

| | | | | | Peptide Largest Impurity by Peak | | | |
|---|---|---|---|---|---|---|---|---|
| Days of | Peptide Total Purity (%) | | | | Area (%) | | | |
| Incubation | Vial 1 | Vial 2 | Average | STDEV | Vial 1 | Vial 2 | Average | STDEV |
| 1 | 99.8 | 99.7 | 99.8 | 0.0 | 0.22 | 0.26 | 0.24 | 0.03 |
| 2 | 99.4 | 99.6 | 99.5 | 0.2 | 0.33 | 0.28 | 0.31 | 0.04 |
| 3 | 99.9 | 99.5 | 99.7 | 0.2 | 0.14 | 0.28 | 0.21 | 0.10 |
| 4 | 99.7 | 99.7 | 99.7 | 0.0 | 0.30 | 0.26 | 0.28 | 0.03 |
| 5 | 99.8 | 99.8 | 99.8 | 0.0 | 0.23 | 0.25 | 0.24 | 0.01 |
| 6 | 99.3 | 99.4 | 99.4 | 0.1 | 0.23 | 0.38 | 0.31 | 0.11 |
| 7 | 99.4 | 99.5 | 99.5 | 0.1 | 0.40 | 0.31 | 0.36 | 0.06 |
| 8 | 99.3 | 99.4 | 99.4 | 0.1 | 0.68 | 0.57 | 0.63 | 0.08 |
| 9 | 100.0 | 100.0 | 100.0 | 0.0 | 0.00 | 0.00 | 0.00 | 0.00 |
| 10 | 99.6 | 96.6 | 98.1 | 2.1 | 0.40 | 3.43 | 1.92 | 2.14 |

“As-sold” stability studies are defined as those studies involving formulation stored within a closed (i.e., capped) vial, placed at specific storage and accelerated temperature conditions (i.e., 5° C., 25° C., 40° C., and/or 50° C.) for specified amounts of time. As-sold stability studies were performed on formulations that had positive results from the in vitro screening rounds. Formulations were manufactured and stored at 5° C., 25° C., and 35° C. Table 18 shows the formulations that were tested. Results for formulations #5 and #6 (containing GLP-1 and EDTA) showed after 56 days of storage the recovery of GLP-1 was 100% at 5° C., and >97% at 25° C. and >90% at 35° C.

TABLE 18
Summary of Formulations Assayed for As-sold Stability

| | | | | | | Citrate | | | | |
|---|---|---|---|---|---|---|---|---|---|---|
| | GLP-1 | DDPC | EDTA | Mg EDTA | MβCD | Buffer | Lactose | Sorbitol | | |
| # | mg/mL | mg/mL | mg/mL | mg/mL | mg/mL | (mM) | (mM) | (mM) | BAK (%) | pH |
| 1 | 5 | 1 | 1 | 0 | 45 | 10 | 25 | 100 | 1 | 3.5 |
| 2 | 5 | 1 | 1 | 0 | 45 | 10 | 0 | 0 | 1 | 3.5 |
| 3 | 5 | 0 | 0 | 0 | 45 | 10 | 0 | 0 | 1 | 3.5 |
| 4 | 5 | 1 | 0 | 0 | 45 | 10 | 0 | 0 | 1 | 3.5 |
| 5 | 5 | 0 | 1 | 0 | 0 | 10 | 0 | 0 | 1 | 3.5 |
| 6 | 5 | 0 | 10 | 0 | 0 | 10 | 0 | 0 | 1 | 3.5 |
| 7 | 5 | 0 | 0 | 10 | 0 | 10 | 0 | 0 | 1 | 3.5 |
| 8 | 5 | 0 | 0 | 0 | 0 | 10 | 0 | 0 | 1 | 3.5 |
| 9 | 5 | 0 | 0 | 10 | 0 | 10 | 0 | 0 | 1 | 5.2 |

An as-sold stability assay was performed on the same formulation batch made for the in-use study (5 mg/mL GLP-1, 10 mg/mL EDTA, 10 mM Citrate Buffer (pH 3.5), and no preservative). The formulation was stored at 5° C. The as-sold stability results are shown below in Tables 19 through 20.

TABLE 19
As-sold Stability, GLP-1 Recovery at 5° C. Storage

| | Peptide concentration | | | | Peptide recovery | | | |
|---|---|---|---|---|---|---|---|---|
| Days of | (ug/mL) | | | | (%) | | | |
| incubation | Vial 1 | Vial 2 | Average | STDEV | Vial 1 | Vial 2 | Average | STDEV |
| 9 | 4704.3 | 4706.0 | 4705.2 | 1.2 | 94.1* | 94.1* | 94.1* | 0.0* |
| 16 | 4625.3 | 4588.0 | 4606.6 | 26.4 | 98.3^{†} | 97.5^{†} | 97.9^{†} | 0.6^{†} |
| 30 | 4813.6 | 4650.4 | 4732.0 | 115.5 | 102.3^{†} | 98.8^{†} | 100.6^{†} | 2.5^{†} |
| 44 | 4676.0 | 4635.2 | 4655.6 | 28.8 | 99.4^{†} | 98.5^{†} | 98.9^{†} | 0.6^{†} |

TABLE 20
As-sold Stability, GLP-1 Purity at 5° C. Storage

| | | | | | Peptide Largest Impurity by Peak | | | |
|---|---|---|---|---|---|---|---|---|
| Days of | Peptide Total Purity (%) | | | | Area (%) | | | |
| incubation | Vial 1 | Vial 2 | Average | STDEV | Vial 1 | Vial 2 | Average | STDEV |
| 9 | 99.8 | 99.8 | 99.8 | 0.0 | 0.13 | 0.19 | 0.16 | 0.04 |
| 16 | 98.3 | 100.0 | 99.2 | 1.2 | 0.54 | 0.00 | 0.27 | 0.38 |
| 30 | 99.8 | 99.9 | 99.9 | 0.1 | 0.19 | 0.11 | 0.15 | 0.06 |
| 44 | 99.6 | 99.9 | 99.7 | 0.2 | 0.30 | 0.14 | 0.22 | 0.11 |

### Example 7

### In Vivo Pharmacodynamics of Intranasal GLP-1 Formulations

Three studies compared the pharmacodynamic (PD) actions of GLP-1 (7-36)amide and synthetic exendin-4 (exenatide) on blood glucose following intranasal (IN) administration of GLP-1 in the presence and absence of DPP-IV inhibitor or subcutaneous injection (SQ) of exenatide. The studies were done in the ZDF rat model of the oral glucose tolerance test (OGTT) used in human studies.

Pharmacodynamic actions were evaluated by monitoring blood glucose and blood insulin levels. Glucose concentration in blood was determined using a Synchron CX4 analyzer and appropriate Glucose Reagent Kit (Beckman Coulter, Brea, Calif. USA). Pharmacokinetic parameters were determined using WinNonlin software (Pharsight Corporation, Version 5.01, Mountain View, Calif.). Insulin concentration in the blood was determined by ELISA.

Acetaminophen concentration in blood was determined using a Synchron CX4 analyzer and Acetaminophen Reagent Kit (Beckman Coulter, Brea, Calif. USA). Acetaminophen was used as a marker of gastric emptying because acetaminophen generally has negligible absorption from the stomach. The time to peak concentration (Tₘₐₓ) and peak concentrations (Cₘₐₓ) after dose administration reflects the time at which gastric emptying occurs, and the profile of gastric emptying (e.g., absorption into the systemic circulation after release from the stomach) reflects the duration of emptying. The AUC reflects the total exposure. Acetaminophen was administered in Study 2 and Study 3 to monitor gastric emptying.

### Study 1

The overall study design for evaluation of blood glucose and insulin values in Study 1 is outlined in Table 21.

TABLE 21
Study 1 Pharmacodynamic (PD) Study Design and Group Designations
for Determination of Blood Glucose and Insulin

| | | | | | GLP-1 Dose | |
|---|---|---|---|---|---|---|
| | No. of | | | Dose level | Conc. | Dose relative |
| Group | Animals* | Treatment | Route | (μg/kg) | (μg/ml) | to OGTT |
| 1 | 5 | Saline | IN | 250 | 0 | −10 minutes |
| 2 | 5 | GLP1: F1 | IN | 250 | 5000 | −10 minutes |
| 3 | 5 | GLP1: F1 | IN | 250 | 5000 | +20 minutes |
| 4 | 5 | GLP1: F2 | IN | 250 | 5000 | −10 minutes |
| 5 | 5 | GLP1: F2 | IN | 250 | 5000 | +20 minutes |
| 6 | 5 | GLP1: F3 | IN | 250 | 5000 | −10 minutes |
| 7 | 5 | GLP1: F3 | IN | 250 | 5000 | +20 minutes |
| 8 | 5 | exenatide | SQ | 3.0 | 5 | −10 minutes |

*Male ZDF Rats

Formulations for GLP-1 treated rats in Study 1 were F110 mg/mL EDTA, 10 mM Citrate buffer (pH 3.5) NO INHIBITOR; F2=10 mg/mL EDTA, 10 mM Citrate buffer (pH 3.5) and 25 mM H-Lys(4-nitro-Z) pyrrolidide (inhibitor is commercial available from Bachem)); and F3=10 mg/mL EDTA, 10 mM Citrate buffer (pH 3.5) and 25 mM L-Proline-boroproline (Pro-boropro was synthesized based on information from Patent Application 2004/0229820 A1, Bachovchin, Willia, W. et al.).

The PD results for Study 1 are shown in Table 22. The AUC(0-150) of glucose for rats treated with GLP-1 formulations (w/and w/o DPP-IV inhibitor) was ˜15% lower then exenatide and placebo treated rats. In Study 1, the glucose was given at 2 g/kg, whereas in subsequent studies glucose was given at 1 g/kg. As a result, in Study 1, the observed glucose reduction was relatively lower than the other studies (e.g., AUC(0-150) of 0-16%) because the challenge of glucose was higher. For Study 1, acetaminophen was not dosed to monitor gastric emptying.

TABLE 22
Study 1 Calculated % Reduction in Glucose AUC for GLP-1 Compared to Placebo and Exenatide

| | | % reduction | | % reduction | | % reduction |
|---|---|---|---|---|---|---|
| | Glucose | Glucose | Glucose | Glucose | Glucose | Glucose |
| Group | AUC(0-60) | AUC(0-60) | AUC(0-120) | AUC(0-120) | AUC(0-150) | AUC(0-150) |
| Placebo | 22155 | 0 | 42915 | 0 | 49775 | 0 |
| Form 1; +20 min | 19120 | 14 | 36250 | 16 | 43975 | 12 |
| Form 1; −10 min | 17525 | 21 | 35015 | 18 | 41930 | 16 |
| Form 2; +20 min | 19003 | 14 | 34723 | 19 | 41892 | 16 |
| Form 2; −10 min | 14193 | 36 | 32673 | 24 | 42167 | 15 |
| Form 3; +20 min | 18703 | 16 | 34603 | 19 | 42252 | 15 |
| Form 3; −10 min | 14615 | 34 | 32945 | 23 | 42095 | 15 |
| Exenatide | 18815 | 15 | 39245 | 9 | 50190 | −1 |

There was no difference in blood glucose between the GLP-1 formulations when administered +20 min after oral glucose dose. There was a difference in blood glucose profile between the GLP-1 formulations with inhibitor (F2 and F3) vs. without inhibitor (F1) when administered pre dose, −10 mins. Both formulations containing a DPP-IV inhibitor (F2 and F3) resulted in a delay in Cₘₐₓ (from 45 to about 120 minutes). Significant levels of GLP-1 delayed gastric emptying; PK rabbit study showed a 5-fold higher % BA for GLP-1 formulation containing DPP-IV inhibitor than without. In Study 2, acetaminophen was added to a formulation containing a DPP-IV inhibitor to compare gastric emptying in a GLP-1 formulation without inhibitor.

Insulin and Glucose measurements for Placebo at −10 minutes, GLP-1 F1 (without inhibitors) at −10 minutes, and GLP-1 F1 (without inhibitors) at +20 minutes data shows an insulin increase greater with the GLP-1 F1 when administered at either −10 and +20 minutes relative to placebo.

### Study 2

PD actions of GLP-1 (7-36 amide) in the presence and absence of DPP-IV inhibitor on blood glucose following intranasal instillation (GLP-1) in a rat model of the OGTT were assayed in Study 2. The overall study design for evaluation of blood glucose and insulin values is outlined in Table 23. The Study 2 OGTT was conducted as a 1 g/kg bolus dose of a glucose solution administered by oral gavage. Acetaminophen, at a dose of 100 mg/kg, was co-administered within the glucose solution. For timing purposes the OGTT was designated as time 0 minutes.

TABLE 23
Study 2 Pharmacodynamic OGTT Design and Group Designations
for Determination of Blood Glucose and Insulin

| | | | | | GLP-1 Dose | |
|---|---|---|---|---|---|---|
| | No. of | | | Dose level | Conc. | Dose relative |
| Group | Animals* | Treatment | Route | (μg/kg) | (μg/ml) | to OGTT |
| 1 | 5 | Saline | IN | 0 | 0 | −10 minutes |
| 2 | 5 | GLP1: F1 | IN | 100 | 2000 | −10 minutes |
| 3 | 5 | GLP1: F1 | IN | 250 | 5000 | −10 minutes |
| 4 | 5 | GLP1: F1 | IN | 1000 | 20000 | −10 minutes |
| 5 | 5 | GLP1: F3 | IN | 25 | 500 | −10 minutes |
| 6 | 5 | GLP1: F3 | IN | 100 | 2000 | −10 minutes |
| 7 | 5 | GLP1: F3 | IN | 250 | 5000 | −10 minutes |

*ZDF Rat

Formulations used in Study 2 included: F1=10 mg/mL EDTA, 10 mM citrate buffer (pH 3.5) NO INHIBITOR; F3=10 mg/mL EDTA, 10 mM citrate buffer (pH 3.5) and 25 mM L-proline-boroproline. Blood glucose was evaluated at the following time points: preOGTT and 5, 10, 15, 30, 45, 60, 75, 90, 120, 180 and 240 minutes post-OGTT. Blood insulin was evaluated at the following time points: preOGTT and 15, 30, 60, 120 and 240 minutes post-OGTT. APAP blood levels were evaluated at the following time points: preOGTT and 5, 10, 15, 30, 45, 60, 75, 90, 120, and 180 minutes post-OGTT. Results from Study 2 show that there was a reduction in glucose AUC (baseline corrected) compared to the control, especially for formulations without inhibitor (F1). Both formulations (F1 and F3) with and without inhibitor increase insulin after dosing. IN dose of 100 ug/mL GLP-1 showed that in the presence of inhibitor, gastric emptying was delayed. Increasing the IN GLP-1 dose also affected gastric emptying even without the presence of inhibitor. When the gastric emptying was delayed due to high dose or the presence of inhibitor, the absorption of glucose was delayed. Table 24 shows the difference in glucose AUC's for treatment groups compared to Placebo.

TABLE 24
Summary of Differences in Glucose AUC

| | | Difference in |
|---|---|---|
| | Difference in | Glucose AUClast |
| | Glucose AUClast | (baseline corrected) |
| Group | from Placebo | from Placebo |
| 100 μg/mL GLP1: F1 | 0.83 | 0.64 |
| 250 μg/mL GLP1: F1 | 0.89 | 0.79 |
| 1000 μg/mL GLP1: F1 | 0.89 | 0.71 |
| 25 μg/mL GLP1: F3 | 0.99 | 0.91 |
| 100 μg/mL GLP1: F3 | 0.92 | 0.82 |
| 250 μg/mL GLP1: F3 | 1.03 | 0.99 |

### Study 3

In Study 3, the pharmacodynamic (PD) actions of GLP-1 (7-36 amide) without inhibitor and exenatide on blood glucose following intranasal instillation (GLP-1 or saline) or subcutaneous injection of exenatide in a rat model of the OGTT were assayed. In Study 3, a GLP-1 formulation that does not have a DPP-IV inhibitors was used (F1=10 mg/mL EDTA, 10 mM citrate buffer (pH 3.5) NO INHIBITOR). The main goal of Study 3 was to compare GLP-1 F1 (IN) to exenatide (SQ). PD was evaluated by monitoring blood glucose and blood insulin levels. Acetaminophen was administered to monitor gastric emptying (from Study 2).

In Study 3 the OGTT was conducted using a 1 g/kg bolus dose of a glucose solution administered by oral gavage. When relevant, acetaminophen, at a dose of 100 mg/kg, was co-administered within the glucose solution. For timing purposes, the OGTT was designated as time 0 minutes. Timing for dose administration of treatment was relative to the OGTT. The single treatment dose was administered at −10 minutes. Twice dosing was conducted with the first dose at −10 minutes and the second dose at +35 minutes. Therefore, dose administration for saline, GLP-1, or exenatide occurred at −10 minutes, +35 minutes, or −10 and +35 minutes, relative to the OGTT.

Blood was collected for pharmacodynamic analysis, including AUC0-240 and Cmax calculations. Blood Glucose was evaluated at the following time points: preOGTT and post-OGTT (5, 15, 30, 45, 60, 75, 90, 120, 180 and 240 minutes). Blood Insulin was evaluated preOGTT and post-OGTT (5, 15, 30, 45, 60, 75, 90, 120 and 180 minutes). Acetaminophen blood levels were evaluated preOGTT and post-OGTT (15, 30, 45, 60, 75, 90, 120, and 180 minutes). The overall design for Study 3 evaluation of blood glucose and insulin values is outlined in Table 25. The overall design for evaluation of gastric emptying is outlined in Table 26.

TABLE 25
Study 3 Pharmacodynamic Study Design and Group Designations
for Determination of Blood Glucose and Insulin

| | No. of | | | Dose level | Dose administration |
|---|---|---|---|---|---|
| Group | Animals* | Treatment | Route | (μg/kg) | relative to OGTT |
| 1 | 5 | Saline | IN | 0 × 2 | −10 minutes/+35 |
| | | (Placebo) | | | minutes |
| 2 | 5 | GLP-1: F1 | IN | 100 × 1 | −10 minutes, no |
| | | | | | further dosing |
| 3 | 5 | GLP-1: F1 | IN | 100 × 2 | −10 minutes/+35 |
| | | | | | minutes |
| 4 | 5 | Exenatide | SQ | 0.6 × 1 | −10 minutes, no |
| | | | | | further dosing |

*Male ZDF Rats

TABLE 26
Study 3 Pharmacodynamic Study Design and Group
Designations for Evaluation of Gastric Emptying

| | No. of | | | Dose level | Dose administration |
|---|---|---|---|---|---|
| Group | Animals* | Treatment | Route | (mg/kg) | relative to OGTT |
| 1 | 5 | Saline | IN | 0 | −10 minutes |
| | | (Placebo) | | | |
| 2 | 5 | GLP-1: F1 | IN | 100 | −10 minutes |
| 3 | 5 | GLP-1: F1 | IN | 1000 | −10 minutes |

*Male ZDF Rats

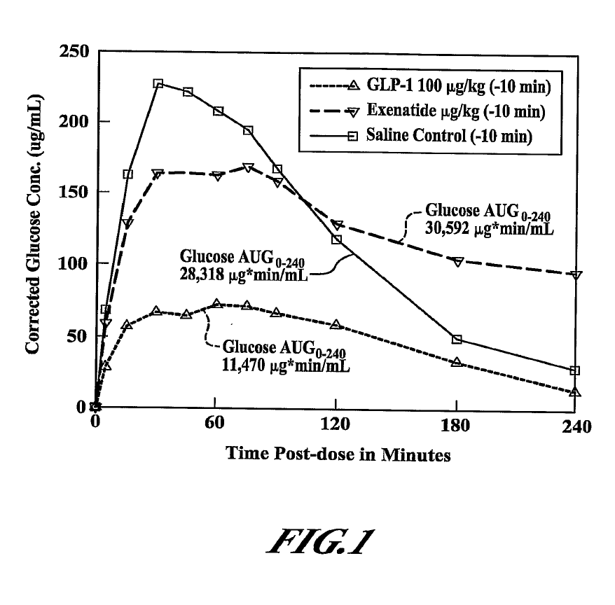

The results of Study 3 show dramatic reduction (˜60%) of corrected blood glucose AUC₀₋₂₄₀ after dosing GLP-1 intranasal compared to Saline or SQ administration of Exenatide. AUC₀₋₂₄₀ and Cₘₐₓ values are shown in Table 27. The change in glucose concentration corrected for endogenous glucose is shown in FIG. 1.

These pharmacodynamic results show the surprising and unexpected discovery that intranasal administration of pharmaceutical formulations of GLP-1 resulted in a decreased glucose concentration in the blood.

TABLE 27
Pharmacodynamic Data for Blood Glucose
Pharmacokinetic Parameters Following Administration of GLP-1,
Exenatide, and Saline in Rats Given an Oral Glucose Tolerance Test

| | | | AUC₀₋₂₄₀ |
|---|---|---|---|
| | | | (corrected for |
| | AUC₀₋₂₄₀ | Cₘₐₓ | endogenous glucose) |
| | (μg*min/mL) | (μg/mL) | (μg*min/mL) |
| Saline (IN) | 59,352 | −356 | 28,318 |
| Exenatide (SQ) | 67,832 | −277 | 30,592 |
| GLP-1 (IN) | 45,550 | −209 | 11,470 |

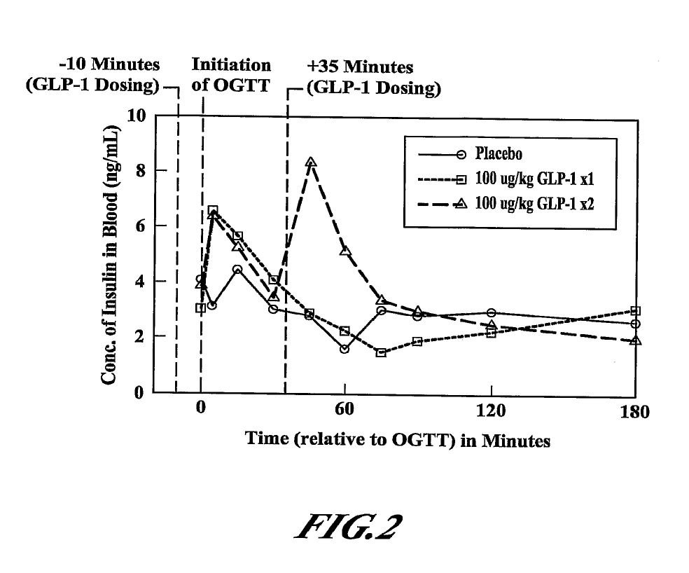

The group mean insulin data for Study 3 GLP-1 treatment groups and placebo are shown in FIG. 2. Insulin levels in saline-treated (placebo) rats show a slight decrease following the OGTT. With a single dose of 100 μg/kg GLP-1 at −10 minutes, there is a marked increase in blood insulin levels in response to an OGTT. The administration of GLP-1 at −10 minutes and +35 minutes results in an insulin spike immediately following each dose. This pattern indicates GLP-1 is responsible for the release of insulin in response to elevated blood glucose.

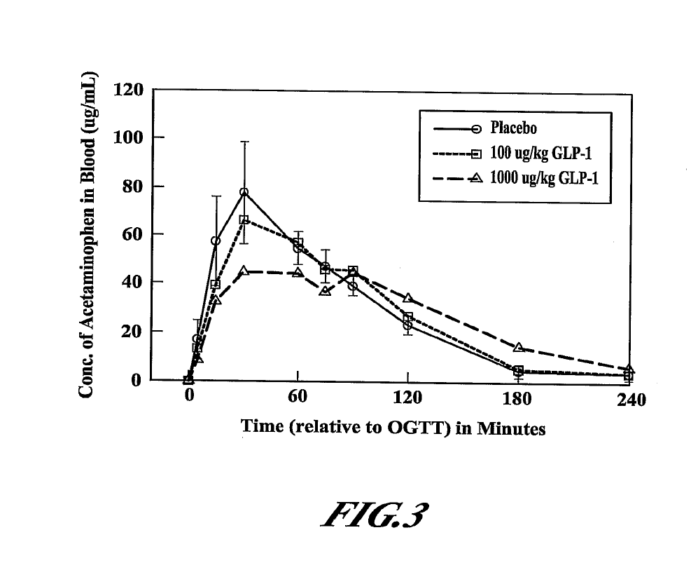

The group mean acetaminophen data for GLP-1 treatment groups and placebo (with standard deviation for the placebo group) are displayed in FIG. 3. In Study 3, determination of total exposure (AUC) indicated there was less than a 3% difference in the total amount of acetaminophen absorbed into the systemic circulation among the three treatment groups. The AUC values were 6804 μg*min/kg, 6672 μg*min/kg, and 6951 μg*min/kg for the saline control (placebo), 100 μg/kg GLP-1, and 1000 μg/kg GLP-1 groups, respectively. However, the profile of absorption was different. In the placebo treated rats, Tₘₐₓ for acetaminophen was 30 minutes post-dose; with a group mean Cₘₐₓ of 78 μg/mL with a standard deviation of +21 μg/mL. A slightly lower Cₘₐₓ, 66±21 ug/mL, was noted at 30 minutes post-dose for the 100 μg/kg GLP-1 group; however, concentrations of acetaminophen between this group and the control group were similar for most time points evaluated. The Cₘₐₓ for acetaminophen was lower, 45±30 ug/mL, in the 1000 μg/kg GLP-1 group. In addition, the Tₘₐₓ appeared to occur between 30 and 90 minutes post-dose, and blood levels of acetaminophen were noticeably higher at the later time points. The results for the 1000 μg/kg GLP-1 group are consistent with a delay in gastric emptying and a more prolonged profile for gastric emptying following a high dose of GLP-1.

The results of Study 3 show that GLP-1 without inhibitor lowers glucose significantly and that the dose can be administered pre- or post-meal. The IN GLP-1 formulation lowered glucose AUC while dosing Exenatide SQ in ZDF rats at either 0.6 or 3 ug/kg did not.

### Summary of PD Results

These pharmacodynamic results show the surprising and unexpected discovery that intranasal administration of pharmaceutical formulations of GLP-1 resulted in a decreased glucose concentration in the blood. Further, the blood insulin concentration pattern indicates GLP-1 is the factor responsible for the release of insulin in response to elevated blood glucose. The results for the acetaminophen study are consistent with a delay in gastric emptying and a more prolonged profile for gastric emptying following a high dose of GLP-1 even in the absence of DPP-IV inhibitor. A lower dose of GLP-1, which was also effective in lower blood glucose, did not impact gastric emptying. Decreasing the glucose concentration in the blood especially through increased insulin levels is an effective treatment for Type II Diabetes. Further, delay in gastric emptying may increase satiety and promote weight-loss. These data support the efficacy of the GLP-1 intranasal formulation described in these assays for use in the treatment of metabolic disorders such as obesity and diabetes.

### Example 8

### Synthetic Exendin-4 (Exenatide) Transmucsal Formulations with Enhancers

A variety of excipients were tested for in vitro optimization of transmucosal exenatide formulations. Transmucosal exenatide formulations were generated by combining exenatide and excipients (including permeation enhancers, solubolizers, surfactants, chelators, stabilizers, buffers, tonicifiers, and preservatives).

Multiple rounds of formulation screening were performed and divided into two series, A and B. Series A focused on changing the excipient concentrations of solubolizers (Me-β-CD), surfactants (DDPC), chelators (EDTA), and stabilizers (gelatin). Buffers such as citrate buffer, tartrate buffer, and glutamate (MSG) were also tested. Series B screened alternative excipients for their potential to enhance exenatide permeation. Various concentrations of potential permeation enhancers including cyclodextrins, glycosides, fatty acids, phosphatidylcholines, GRAS compounds, PN159, gelatin, and others were tested. In addition to screening potential permeation enhancers, varing concentrations of buffer (citrate Buffer, tartrate Buffer) and tonicifier/stabilizer excipients (mannitol, NaCl) were also screened. Preservatives such as sodium benzoate (NaBz) and benzalkonium chloride (BAK) were tested. Table 28 lists the excipients tested in the in vitro screening. Out of 372 unique formulations that were tested, eleven formulations were recommended for use in preclinical in vivo rabbit PK studies, see Table 29.

TABLE 28
Excipients Tested in In Vitro Exenatide Formulation Optimization

| | | Concentration | |
|---|---|---|---|
| Excipient | Function | Range Tested | |
| Citrate Buffer | Buffer/Chelator/Co-preservative | 20 mM, | pH 4.5 |
| Tartrate Buffer | Buffer | 30 mM, | pH 4.5 |
| Mannitol | Tonicifier/Stabilizer | 50-200 | mM |
| Sodium Chloride | Tonicifier/Stabilizer | 0-50 | mM |
| Sodium Benzoate | Preservative | 0-5 | mg/mL |
| Benzalkonium Chloride | Preservative | 0-9 | mg/mL |

Series A Excipients

| Me-β-CD | Solubilizer/Stabilizer/Enhancer | 0-90 | mg/mL |
|---|---|---|---|
| EDTA | Chelator/Stabilizer/Enhancer/ | 0-10 | mg/mL |
| | Co-preservative | | |
| DDPC | Solubilizer/Enhancer | 0-2 | mg/mL |
| Gelatin | Stabilizer/Viscosity Enhancer | 0-10 | mg/mL |
| Series B Excipients | Class | | |
| DMe-β-CD | Cyclodextrins | 20-50 | mg/mL |
| HP-β-CD | Cyclodextrins | 20-50 | mg/mL |
| β-CD | Cyclodextrins | 10-20 | mg/mL |
| n-Decyl-β-D-maltopyranoside | Glycosides | 2.5-10 | mg/mL |
| n-Dodecyl-β-D-maltopyranoside | Glycosides | 2.5-10 | mg/mL |
| n-Tetradecyl-β-D-maltopyranoside | Glycosides | 2.5-10 | mg/mL |
| n-Octyl-β-D-maltopyranoside | Glycosides | 10-20 | mg/mL |
| n-Hexadecyl-β-D-maltopyranoside | Glycosides | 2.5-10 | mg/mL |
| n-Octyl-β-D-galactopyranoside | Glycosides | 5-10 | mg/mL |
| Octyl-β-glucopyranoside | Glycosides | 5-10 | mg/mL |
| Octyl-α-glucopyranoside | Glycosides | 5-7.5 | mg/mL |
| n-Heptyl-β-D-glucopyranoside | Glycosides | 2.5-10 | mg/mL |
| Dodecanoylsucrose | Glycosides | 1-5 | mg/mL |
| Decanoylsucrose | Glycosides | 1-5 | mg/mL |
| | | Concentration | |
| Excipient | Function | Range Tested | |
| Sodium Caprate (10) | Unsaturated fatty acids | 5-50 | mg/mL |
| Sodium Caprylate (8) | Unsaturated fatty acids | 20-100 | mg/mL |
| Phosphotidyl chorine | phosphatidylcholines | 0.177-1.77 | mmol |
| Dimyristoyl Glycero | phosphatidylcholines | 0.177-1.77 | mmol |
| Phosphatidylcholine (14:0) DMPC | | | |
| Dilauroyl Glycero | phosphatidylcholines | 0.177-1.77 | mmol |
| Phosphatidylcholine (12:0) DLPC | | | |
| Di Nonanoyl Glycero | phosphatidylcholines | 0.177-1.77 | mmol |
| Phosphatidylcholine (9:0) Di Non- | | | |
| PC | | | |
| Dipalmitoyl Glycero | phosphatidylcholines | 0.177-1.77 | mmol |
| Phosphatidylglycerol (16:0) DPPG | | | |
| Dimyristoyl Glycero | phosphatidylcholines | 0.177-1.77 | mmol |
| Phosphatidylglycerol (14:0) DMPG | | | |
| Palmitoyl-DL-Carnitine | Other | 1-5 | mg/mL |
| Sodium Glycocholate | Other | 1-10 | mg/mL |
| S nitroso-N-acetyl-penicillamine | Other | 0.2-1 | mg/mL |
| Cremephor EL | Other | 1-5 | mg/mL |
| PN159 | Other | 20-100 | mg/mL |
| recombinant high molecular weight | Other | 2.5 | mg/mL |
| gelatin | | | |
| recombinant low molecular weight | Other | 2.5 | mg/mL |
| gelatin | | | |
| Oleic acid | GRAS | 1-3 | mg/mL |
| Lecithin | GRAS | 0.7 | mg/mL |
| Ethanol | GRAS | 1-20 | mg/mL |
| Tween 80 | GRAS | 50 | mg/mL |
| propylene glycol | GRAS | 100 | mg/mL |
| EDTA alone | GRAS | 2.5-10 | mg/mL |

TABLE 29
Transmucosal Exenatide Formulations Recommended for Pre-clinical Studies

| | Exenatide | Me-β-CD | DDPC | EDTA | Gelatin | Buffer pH 4.5 | Tonicifier | NaBz | Dose |
|---|---|---|---|---|---|---|---|---|---|
| Sample | (mg/ml) | (mg/ml) | (mg/ml) | (mg/ml) | (mg/ml) | (mM) | (mM) | (mg/ml) | vol |
| | | | | | | Citrate | Mannitol | | |
| AKL-225-126-2 | 3 | 40 | 1 | 2.5 | 0 | 20 | 80 | 1 | full |
| JW-239-9-21 | 3 | 80 | 2 | 5 | 0 | 20 | 40 | 0 | full |
| | | | | | | Tartrate | NaCl | | |
| JW-239-126-3 | 3 | 40 | 1 | 2.5 | 0 | 30 | 37 | 0 | full |
| JW-239-126-7 | 3 | 80 | 2 | 5 | 0 | 30 | 11 | 0 | full |
| JW-239-126-14 | 3 | 80 | 2 | 5 | 2.5 | 30 | 0 | 0 | full |
| JW-239-126-15 | 6 | 40 | 1 | 2.5 | 0 | 30 | 34 | 0 | full |
| JW-239-126-19 | 6 | 80 | 2 | 5 | 0 | 30 | 8 | 0 | half |
| JW-239-126-24 | 6 | 80 | 2 | 5 | 2.5 | 30 | 0 | 0 | half |
| JW-239-126-19 | 6 | 80 | 2 | 5 | 0 | 30 | 8 | 0 | full |
| JW-239-126-24 | 6 | 80 | 2 | 5 | 2.5 | 30 | 0 | 0 | full |
| AKL-310-27-12 | 6 | 0 | 0 | 10 | 0 | 30 | 20 | 0 | full |

### Example 9

### Transmucosal Exenatide Formulations Induce Opening of Tight Junctions In Vitro

In vitro TER, LDH, MTT, and permeation assays were performed for exenatide formulations as described in the protocols in Example 2 above.

For all exenatide formulations containing enhancers, TER was reduced from approximately 350-700 ohms×cm² to approximately 5-20 ohms×cm² after the sixty (60) minute incubation period. All exenatide formulations, with the exception of controls, contained EDTA. As a calcium chelator, EDTA is known to open tight junctions by scavenging calcium. In a static environment like the in vitro tissue culture system used here, the removal of calcium from solution leads to significant tight junction opening. No reduction in TER was observed in the exenatide plus glutamate control (MSG) containing only exenatide in glutamate buffer with sodium chloride as a tonicifier. The exenatide plus glutamate control indicates that opening tight junctions is not an inherent characteristic of exenatide itself. The TER of inserts after sixty (60) minutes exposure to the glutamate control is similar to that of inserts exposed to media for sixty (60) minutes. The triton X control was the lowest possible TER, which results from killing the cell barrier as expected.

To verify that TER reduction by the exenatide formulations resulted from tight junction modulation by the enhancers and not cell death, LDH and MTT assays were performed using the same cell line, MatTek Corp., as used in the TER assays. Exenatide formulations did not show a significant increase in cytotoxicity as measured by % LDH. Exenatide formulations had less than 20% LDH loss. Similarly, media control did not show cytotoxicity. In contrast, Triton X control treated group showed significant toxicity, as expected. Cell viability was assessed using the MTT assay (MTT-100, MatTek kit). Exenatide formulations did not show a significant increase in cytotoxicity as measured by the % MTT with the exception of three formulations, JW-239-126-14, JW-239-126-19, and JW-239-126-24, which had around 50% MTT. Otherwise, exenatide formulations showed viablility greater than 80% MTT. Similarly, media control did not show cytotoxicity. In contrast, Triton X control treated group showed significant toxicity as expected. Results of the permeation study are show in Table 30.

TABLE 30
Transmucosal Exenatide Formulations from Previous Studies

| | | Fold | | | | | | |
|---|---|---|---|---|---|---|---|---|
| | | perm rel | Permeation | | MTT | | LDH | |
| | load | to MSG | | % std | | % std | | % std |
| Sample | vol | ctrl | % | dev | % | dev | % | dev |
| AKL-225-126-2 | full | 346 | 6.0 | 1.6 | 103.2 | 16.5 | 2.5 | 2.0 |
| JW-239-9-21 | full | 501 | 7.2 | 1.6 | 73.3 | 18.7 | 7.2 | 4.1 |
| JW-239-126-3 | full | 365 | 8.6 | 1.7 | 90.6 | 20.7 | 3.4 | 1.6 |
| JW-239-126-7 | full | 430 | 8.2 | 4.7 | 84.5 | 17.9 | 6.1 | 2.9 |
| JW-239-126-14 | full | 829 | 16.7 | 8.5 | 52.0 | 11.0 | 6.3 | 2.0 |
| JW-239-126-15 | full | 361 | 13.4 | 14.4 | 112.2 | 20.9 | 3.4 | 4.1 |
| JW-239-126-19 | half | 1426 | 20.6 | 7.1 | 90.1 | 17.9 | 3.5 | 2.8 |
| JW-239-126-24 | half | 2264 | 32.7 | 8.2 | 98.5 | 15.9 | 7.3 | 1.4 |
| JW-239-126-19 | full | 492 | 7.1 | 1.1 | 41.8 | 6.1 | 15.4 | 0.8 |
| JW-239-126-24 | full | 469 | 6.8 | 1.1 | 45.6 | 6.4 | 13.9 | 1.5 |
| AKL-310-27-12 | full | 306 | 4.4 | 1.5 | 101.7 | 11.6 | 10.8 | 0.6 |

Permeation results for the formulations showed 300 to 830 fold increase in permeation relative to a control formulation containing only 3 mg/mL exenatide and monosodium glutamate. Formulations dosed at half the volume showed a 1400 and 2200 fold increase in permeation compared to the control.

### Example 10

### Rabbit PK Results for Transmucosal Exenatide Formulations

Exendin-4 formulations prepared for in vivo testing are shown in Table 31.

TABLE 31
Transmucosal Exenatide Formulations from Previous Studies

| | 2x enh + | | 1x enh + | |
|---|---|---|---|---|
| Formulation: | gel | 2x enh | gel | 1x enh |
| Exendin-4 | 2-6 | 2-6 | 2-6 | 2-6 |
| (mg/ml) | | | | |
| Me-β-CD | 80 | 80 | 40 | 40 |
| (mg/ml) | | | | |
| DDPC (mg/ml) | 2 | 2 | 1 | 1 |
| EDTA (mg/ml) | 5 | 5 | 2.5 | 2.5 |
| Gelatin (mg/ml) | 2.5 | — | 2.5 | — |
| NaCl (mM) | — | — | 25 | 37 |
| BAK (mg/ml) | 0, 0.2 | 0, 0.2 | 0, 0.2 | 0, 0.2 |
| Buffer | 30 mM | 30 mM | 30 mM | 30 mM |
| | KNa | KNa | KNa | KNa |
| | Tartrate | Tartrate | Tartrate | Tartrate |
| pH | 4.7 | 4.7 | 4.7 | 4.7 |

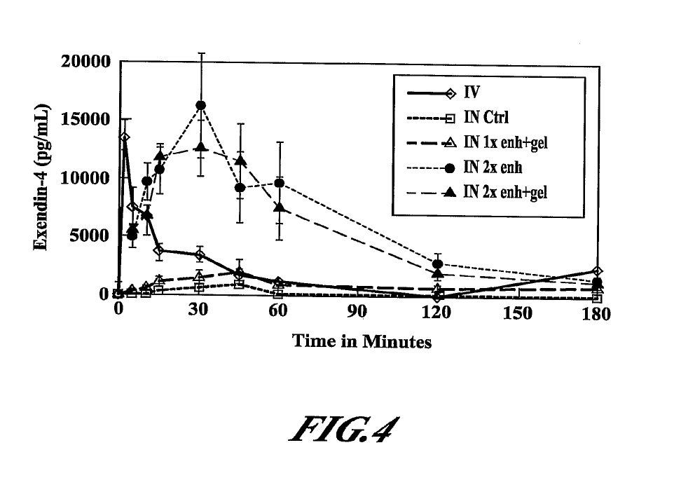

An exendin-4 PK study was performed in rabbits comparing PK results for exendin-4 administered by IV and IN. IN formulations included an IN Control (without enhancers), IN 1× enhancer+gelatin, IN 2× enhancer, and IN 2× enhancer+gelatin (formulations shown in Table 31). The results of the PK study are shown in Table 32 and FIG. 4.

TABLE 32
Pharmacokinetics Results for Exendin-4 in Rabbits

| | Average | | | | |
|---|---|---|---|---|---|
| Time | IV | IN Ctrl | IN 1x enh + gel | IN 2x enh | IN 2x enh + gel |
| 0 | 0 | 79 ± 33 | 436 ± 98 | 271 ± 131 | 353 |
| 1.5 | 13513 ± 1085 | — | — | — | — |
| 5 | 7534 ± 1527 | 81 ± 34 | 376 ± 114 | 5022 ± 1030 | 5505 ± 552 |
| 10 | 6785 ± 1664 | 72 ± 16 | 590 ± 216 | 9724 ± 1608 | 6858 ± 380 |
| 15 | 3807 ± 896 | 350 ± 222 | 1190 ± 430 | 10814 ± 2105 | 11807 ± 911 |
| 30 | 3420 ± 586 | 638 ± 522 | 1557 ± 608 | 16270 ± 4489 | 12614 ± 2396 |
| 45 | 1767 ± 723 | 953 ± 736 | 2070 ± 1000 | 9280 ± 3024 | 11515 ± 3189 |
| 60 | 1262 ± 461 | 171 ± 56 | 971 ± 290 | 9684 ± 3498 | 7534 ± 2699 |
| 120 | 0 | 85 ± 27 | 715 ± 278 | 2946 ± 828 | 2052 ± 603 |
| 180 | 2508 | 82 ± 22 | 882 ± 483 | 1561 ± 236 | 1298 ± 515 |

The PK results showed that IN 2× enh and IN 2× enh+gel were the best performing exendin-4 formulations tested in the rabbit study. Both IN 2× enh and IN 2× enh+gel resulted in greater PK values than IV or IN controls.

### Example 11

### Alternative Transmucosal Exenatide Formulations

Alternative exendin-4 formulations for transmucosal administration were developed to test for the following: 1) increased storage stability, 2) increased bioavailability of exendin-4, and 3) increased pharmacodynamic effect determined by measuring insulin and glucose levels.

Modifications of previously tested exendin-4 formulations were prepared by removal of DDPC and gelatin in all but one formulation. “OEF” was used to refer to formulations containing 80 mg/mL Me-b-CD and 5 mg/mL EDTA (without DDPC or gelatin) in 10 mM acetate buffer. A second change in the previously tested formulations was the addition of arginine to some of the formulations. The OEF formulations are described in Table 33.

TABLE 33
Transmucosal Exenatide Formulations for In Vitro Permeation Studies

| | 2x enh + | OEF + | OEF + arg, | | OEF, 2.8 | OEF, 10 |
|---|---|---|---|---|---|---|
| Formulation: | gel, arg | arg | 30 Acetate | OEF | arg buffer | arg buffer |
| Exendin-4 (mg/ml) | 2-6 | 2-6 | 2 | 2 | 2 | 2 |
| Me-β-CD (mg/ml) | 80 | 80 | 80 | 80 | 80 | 80 |
| DDPC (mg/ml) | 2 | — | — | — | — | — |
| EDTA (mg/ml) | 5 | 5 | 5 | 5 | 5 | 5 |
| Gelatin (mg/ml) | 2.5 | — | — | — | — | — |
| NaCl (mM) | 40 | 45 | 20-50 | 37 | 46 | 41 |
| Arginine (mM) | 10 | 2.8 | 2.8 | — | 2.8 | 10 |
| Buffer | (Arginine) | 10 mM | 30 mM | 10 mM | (Arginine) | (Arginine) |
| | | Acetate | Acetate | Acetate | | |
| pH | 4.7 | 5.25 | 5.25 | 5.5 | 5.25 | 5.25 |

Abbreviations: DDPC = didecanoyl L-α-phosphatidylcholine, EDTA= Edetate disodium dihydrate, Me-β-CD = Random methyl-β-cyclodextrin.

### Percent Permeation

In vitro permeation studies showed that the OEF formulations (without DDPC and gelatin) enhanced permeation of exendin-4 to a greater extent than the exendin-4 formulations previously tested (see previously tested formulations in Table 31) while providing comparable or better cell viability. The permeation results comparing the formulations are shown in Table 34.

TABLE 34
In Vitro Permeation Results for Exendin-4
Formulations After 90 Minutes

| Formulation | Avg % Permeation | Std. Dev. |
|---|---|---|
| No enh Control | 0.21 | 0.03 |
| 1x enh + gel | 5.04 | 1.04 |
| 2x enh | 7.74 | 0.76 |
| 2x enh + gel | 7.41 | 0.05 |
| 2x enh + gel, arg | 8.37 | 0.64 |
| OEF + arg | 10.14 | 0.40 |

### Pharmacokinetic Study

An in vivo pharmacokinetic (PK) study in rabbits demonstrated that formulations “OEF+arg” and “2× enh+gel, arg” when delivered intranasally produced enhanced bioavailability of exendin-4, comparable to or exceeding that of the previously-tested formulations. The mean peak exendin-4 plasma concentration was greatest for the OEF+arg formulation. Table 35 shows a comparison of the mean PK parameters (Tₘₐₓ, Cₘₐₓ, AUC, t_{1/2}, and Kel) for the tested formulations. The coefficient of variance for each PK parameter is shown in Table 36, and the absolute bioavailability (% F) for each intranasal formulation is shown in Table 37.

TABLE 35
Mean PK Parameters for Exendin-4 Formulations

| | Group | Tₘₐₓ | Cₘₐₓ | AUCₗₐₛₜ | AUC_{inf} | t_{1/2} | Kel |
|---|---|---|---|---|---|---|---|
| Formulation | # | (min) | (pg/mL) | (min*pg/mL) | (min*pg/mL) | (min) | (1/min) |
| IV | 1 | 1.5 | 10500 | 189610 | 207110 | 28.0 | 0.026 |
| IN Control | 2 | 67.0 | 3370 | 137520 | 221070 | 31.8 | 0.025 |
| 1x enh + gel | 3 | 35.0 | 1930 | 141210 | 199180 | 89.2 | 0.012 |
| 2x enh | 4 | 34.0 | 14140 | 1074220 | 1193120 | 50.6 | 0.015 |
| 2x enh + gel | 5 | 33.0 | 15000 | 978940 | 1040300 | 35.1 | 0.022 |
| 2x enh + gel, arg | 6 | 47.0 | 11180 | 692050 | 774490 | 25.6 | 0.029 |
| OEF + arg | 7 | 27.0 | 23290 | 1417370 | 1551880 | 36.7 | 0.020 |

TABLE 36
Mean % Coefficient of Variation

| | Group | Tₘₐₓ | Cₘₐₓ | AUCₗₐₛₜ | AUC_{inf} |
|---|---|---|---|---|---|
| Formulation | # | (min) | (pg/mL) | (min*pg/mL) | (min*pg/mL) |
| IV | 1 | 0.0 | 61.6 | 46.0 | 52.0 |
| IN Control | 2 | 99.1 | 164.1 | 156.5 | 119.0 |
| 1x enh + gel | 3 | 53.5 | 79.8 | 80.3 | 75.8 |
| 2x enh | 4 | 73.8 | 52.6 | 51.0 | 43.8 |
| 2x enh + gel | 5 | 38.0 | 36.4 | 49.7 | 52.5 |
| 2x enh + gel, arg | 6 | 96.2 | 72.7 | 88.8 | 94.4 |
| OEF + arg | 7 | 46.5 | 42.7 | 40.2 | 48.4 |

TABLE 37
Absolute Bioavailability (% F) using AUCₗₐₛₜ

| | | AUCₗₐₛₜ | |
|---|---|---|---|
| Formulation | Group # | (min*pg/mL) | % F |
| IV | 1 | 189610 | |
| IN Control | 2 | 137520 | 1.6 |
| 1x enh + gel | 3 | 141210 | 1.7 |
| 2x enh | 4 | 1074220 | 12.6 |
| 2x enh + gel | 5 | 978940 | 11.5 |
| 2x enh + gel, arg | 6 | 692050 | 8.1 |
| OEF + arg | 7 | 1417370 | 16.6 |

Results for the PK study show that OEF+arg had the highest % bioavailability (16.6%). Two other intranasal formulations, 2× enh and 2× ehn+gel, also had significantly enhanced bioavailability compared to m Control (12.6% and 11.5%, respectively).

### Pharmacodynamic Study

An in vivo study in rabbits provided pharmacodynamic (PD) parameters showing that formulations “OEF+arg” and “2× enh+gel, arg” when delivered intranasally produced insulin and glucose responses, comparable to or exceeding that of previously-described formulations (see Table 31). Table 38 includes PD parameters for mean insulin levels (Tₘₐₓ, Cₘₐₓ, and AUC) and Table 39 shows the mean glucose levels (Tₘᵢₙ and Cₘᵢₙ). Surprisingly, the “2× enh+gel, arg” formulation elicited the greatest PD response even though it did not result in the greatest increase in exendin-4 bioavailability in the PK study.

TABLE 38
PD Parameters for Insulin Levels After
Dosing Formulations in Rabbits

| | Group | | Tₘₐₓ | Cₘₐₓ | AUCₗₐₛₜ |
|---|---|---|---|---|---|
| Formulation | # | N | (min) | (μIU/mL) | (min*μIU/mL) |
| IV | 1 | 3 | 15.0 | 40.2 | 490.3 |
| IN Control | 2 | 1 | 60.0 | 8.0 | 60.0 |
| 1x enh + gel | 3 | 1 | 5.0 | 8.5 | 112.5 |
| 2x enh | 4 | 4 | 21.3 | 34.4 | 518.9 |
| 2x enh + gel | 5 | 2 | 17.5 | 38.5 | 247.5 |
| 2x enh + gel, arg | 6 | 2 | 30.0 | 58.5 | 1211.3 |
| OEF + arg | 7 | 3 | 16.7 | 38.3 | 513.5 |

TABLE 39
PD Parameters for Glucose Levels After
Dosing Formulations in Rabbits

| Formulation | Group # | Tₘᵢₙ | % Cₘᵢₙ |
|---|---|---|---|
| IV | 1 | 1.5 | 96 |
| IN Control | 2 | 5 | 95 |
| 1x enh + gel | 3 | N/A | N/A |
| 2x enh | 4 | 5 | 96 |
| 2x enh + gel | 5 | N/A | N/A |
| 2x enh + gel, arg | 6 | 10 | 80 |
| OEF + arg | 7 | 5 | 94 |

### Stability

Accelerated stability studies show that “OEF” formulation variants provide enhanced stability for exendin-4 relative to the “2× enh+gel” formulation. The stability of the “OEF” formulations was comparable to that of commercially available BYETTA®. After four (4) weeks at 40° C., the purity of the “OEF” formulations was 85.8-86.0% while the purity of “2× enh+gel” was 83.7%. After four (4) weeks at 50° C., the purity of the “OEF” formulations was similar to BYETTA® (72.2-72.9% vs. 72.9%) while the “2× enh+gel” formulation had precipitated. No precipitation was observed in the simple formulations.

In summary, the accelerated stability studies demonstrated that the Acetate buffer provided increased physical stability for exendin-4 formulations relative to tartrate buffer. Less precipitation was observed in acetate buffered formulations than in tartrate-buffered formulations. Generally, the mono-ionogenic buffers (acetate, arginine, lactate) provided better stability than poly-ionogenic buffers (tartrate, citrate). The range of pH 4.7-5.5 provided improved chemical stability for exendin-4 as compared to pH≦4.25. Arginine provided slightly enhanced chemical stability. Addition of methionine and aspartic acid decreased the chemical stability of exendin-4. Optimal osmolality for exendin-4 chemical stability was 200-250 mOsm/kg H₂O.

Additionally, accelerated stability studies indicate that “OEF” formulation variants provide enhanced stability for exendin-4 relative to the existing “2× enh+gel” formulation. The stability of the “OEF” formulations is comparable to that of the commercially available BYETTA®. At 40° C., after four weeks, the purity of the “OEF” formulations is 85.8-86.0% while the purity of “2× enh+gel” is 83.7. More dramatically, at 50° C., after four weeks, the purity of the “OEF” formulations is comparable to BYETTA® (72.2-72.9% vs. 72.9%) while the “2× enh+gel” formulation had precipitated.

Although not tested in this study, it is envisioned that viscosity enhancers can be added to the formulations. Preservative can also be included in the formulations, which might include, but are not limited to, benzalkonium chloride, methyl and propyl parabens, and/or chlorobutanol. Examples of other preservatives previously tested which can be included in the formulations to promote antimicrobial effectiveness include the following combinations: 0.033% Methylpapraben+0.017% Propylparaben; 0.18% Methylpapraben+0.02% Propylparaben; 0.10% to 0.50% chlorobutanol; 0.10% to 0.25% chlorobutanol+0.033% Methylpapraben+0.017% Propylparaben; 0.10% to 0.25% chlorobutanol+0.18% Methylpapraben+0.02% Propylparaben; 0.5% benzyl alcohol; 0.5% benzyl alcohol+0.033% Methylpapraben+0.017% Propylparaben; 0.5% benzyl alcohol+0.18% Methylpapraben+0.02% Propylparaben; 0.5% phenylethanol+0.1 to 0.25% chlorobutanol; 0.5% phenylethanol+0.033% Methylpapraben+0.017% Propylparaben; 0.5% phenylethanol+0.18% Methylpapraben+0.02% Propylparaben; 5 mg/ml EDTA; 0.01-0.1% benzalkonium chloride; 7 mg/mL Ethanol; 5 mg/mL benzyl alcohol+2.5 mg/mL phenylethyl alcohol.

Varying concentrations of exenatide can be used to achieve a desired dose, concentrations from 0.5 mg/ml to 6 mg/ml for example. “OEF” and “OEF+arg” formulations may provide enhanced stability and bioavailability for other GRPs, including, but not limited to, other GLP-1 analogs.

An exendin-4 formulation for transmucosal administration with increased shelf life, decreased cost of goods, and increased bioavailability was identified from the OEF improvements to the formulation. Increased shelf life means the commercial product will last longer, allowing for less expired product and reduced manufacturing. Increased bioavailability means potentially increased efficacy and therapeutic utility of the drug product. It could also mean a savings in costs by reducing the amount of API (exendin-4, GLP-1) required for the efficacy of the drug product. Removal of DDPC allows for potentially greater ease in approval of the product by the FDA since DDPC is a novel excipient. It also decreases the time required—for manufacturing the product. Furthermore, DDPC is an expensive excipient and removal significantly reduces the cost of goods associated with the drug product. Removal of gelatin results in a decrease in the time required for manufacturing the product and also decreases the cost to manufacture with the removal of an excipient.

The invention also includes preservative-free GRP formulations. Such formulations do not contain a preservative. In the absence of an antimicrobial excipient, the formulation is filled under sterile conditions into a preservative-free delivery device. A preservative-free exenatide formulation may include formulations such as those shown in Tables 40 and 41. Exenatide concentration may vary from 2-12 mg/ml.

TABLE 40
Preservative-free Formulation for “2x Enhancers + Gelatin”

| | Concentration | |
|---|---|---|
| Component | (mg/mL) | (mM) |
| Exenatide | Depends on formulation potency required | |
| Methyl-β cyclodextrin | 80.0 | ~29.4-30.4** |
| L-Alpha-phosphatidylcholine | 2.0 | 1.77 |
| didecanoyl | | |
| Edetate disodium | 5.0 | 6.72 |
| Potassium sodium tartrate | 7.566 | 26.81 |
| Tartaric acid | 0.479 | 3.19 |
| Gelatin | 2.5 | varies |

| | |
|---|---|
| Purified water or | QS |
| Sterile water for irrigation | |

TABLE 41
Preservative-free Formulation for “1x Enhancers + Gelatin”

| | Concentration | |
|---|---|---|
| Component | (mg/mL) | (mM) |
| Exenatide | Depends on formulation potency required | |
| Methyl-β cyclodextrin | 40.0 | ~29.4-30.4** |
| L-Alpha-phosphatidylcholine | 1.0 | 1.77 |
| didecanoyl | | |
| Edetate disodium | 2.5 | 6.72 |
| Potassium sodium tartrate | 7.566 | 26.81 |
| Tartaric acid | 0.479 | 3.19 |
| Soditim chloride | 1.46 | 25 |
| Gelatin | 2.5 | varies |

| | |
|---|---|
| Purified water or | QS |
| Sterile water for irrigation | |

Other embodiments may vary levels of the permeation enhancing components such as the following: Me-β-CD (20-80 mg/ml), DDPC (0-2 mg/ml), EDTA (2-10 mg/ml), tartrate buffer (0-30 mM), gelatin, and sodium chloride. Other embodiments may contain a different formulation: Me-β-CD (80 mg/ml), EDTA (5 mg/ml), arginine (2.8 or 10 mM) and/or acetate buffer (10 mM), and sodium chloride with a pH of 4.9-5.6. The concentrations of these excipients may vary. Formulations may further contain a viscosity enhancer such as gelatin, hydroxymethylcellulose, carboxymethylcellulose, or carbopol. Varying concentrations of exenatide could be used to achieve a desired dose. Concentrations could range from 0.5 mg/ml to 25 mg/ml, for instance.

### Example 12

### Single Dose Pharmacodynamic (PD) Study of Intranasal Administration of Glucose-regulating Proteins (GRPs) in a Rat Model of the Oral Glucose Tolerance Test (OGTT)

The pharmacodynamic actions of GLP-1 (7-36 amide) and exendin-4 on blood glucose following intranasal instillation in a rat model of the oral glucose tolerance test (OGTT) were tested. Two formulations of exendin-4 were evaluated and a single formulation of GLP-1 was used. The GLP-1 (EDTA-based) formulation (#2) contained GLP-1 (7-36 amide) and the following ingredients: 10 mg/mL disodium EDTA; 10 mM citrate buffer, pH 3.5. The exendin-4 (EDTA-based) formulations (#2, #3, #4, and #5) contained exendin-4 and the following ingredients: 10 mg/mL disodium EDTA and 10 mM arginine buffer, pH 4.0. The exendin-4 (PDF-based) formulations (#6 and #7) contained exendin-4 and the following ingredients: 45 mg/mL Me-β-Cd, 1 mg/mL DDPC, 1 mg/mL disodium EDTA, 100 mM sorbitol, 25 mM lactose, 5 mg/mL CB, and 10 mM arginine buffer, pH 4.0. A summary of the tested formulations is shown in Table 42. The study design and group designations are shown in Table 43.

TABLE 42
Formulations used to Evaluate Intranasal Dosing of Exendin-4 and GLP-1

| ID/Group # | Formulation |
|---|---|
| Saline Control/#1 | Saline (0.9% NaCl) |
| GLP-1 (EDTA-based)/#2 | 2.00 mg/mL GLP-1, 10 mg/mL EDTA, 10 mM Citrate |
| | buffer (pH 3.5) |
| Exendin-4 (EDTA-based)/#3 | 0.04 mg/mL Exendin-4, 10 mg/mL EDTA, 10 mM arginine |
| | buffer, pH 4.0 |
| Exendin-4 (EDTA-based)/#4 | 0.20 mg/mL Exendin-4, 10 mg/mL EDTA, 10 mM arginine |
| | buffer, pH 4.0 |
| Exendin-4 (EDTA-based)/#5 | 0.40 mg/mL Exendin-4, 10 mg/mL EDTA, 10 mM arginine |
| | buffer, pH 4.0 |
| Exendin-4 (PDF-based)/#6 | 0.04 mg/mL Exendin-4, 45 mg/mL Me-β-CD, 1 mg/mL |
| | DDPC, 1 mg/mL EDTA, 100 mM sorbitol, 25 mM lactose, |
| | 5 mg/mL CB, 10 mM arginine buffer, pH 4.0 |
| Exendin-4 (PDF-based)/#7 | 0.20 mg/mL Exendin-4, 45 mg/mL Me-β-CD, 1 mg/mL |
| | DDPC, 1 mg/mL EDTA, 100 mM sorbitol, 25 mM lactose, |
| | 5 mg/mL CB, 10 mM arginine buffer, pH 4.0 |

Abbreviations: Me-β-CD = methyl-beta-cyclodextrin, EDTA = disodium edetate, DDPC = L-α- phosphatidylcholine didecanoyl, and CB = Chorobutanol

TABLE 43
Study Design and Group Designations

| | | | | | | | Dose |
|---|---|---|---|---|---|---|---|
| | | Dose | Dose | Dose | | | Administration |
| Group | Treatment | Level | Conc. | Vol. | | Route of | relative to |
| # | (API) | (μg/kg) | (mg/ml) | (ml/kg) | Formulation | Admin. | OGTT |
| 1 | Saline | 0 | 0 | 0.05 | N/A (Saline) | Intranasal | −10 minutes |
| 2 | GLP-1 | 100 | 2.00 | 0.05 | EDTA-based | Intranasal | −10 minutes |
| 3 | Exendin-4 | 2 | 0.04 | 0.05 | EDTA-based | Intranasal | −10 minutes |
| 4 | Exendin-4 | 10 | 0.20 | 0.05 | EDTA-based | Intranasal | −10 minutes |
| 5 | Exendin-4 | 20 | 0.40 | 0.05 | EDTA-based | Intranasal | −10 minutes |
| 6 | Exendin-4 | 2 | 0.04 | 0.05 | PDF-based | Intranasal | −10 minutes |
| 7 | Exendin-4 | 10 | 0.20 | 0.05 | PDF-based | Intranasal | −10 minutes |

Pharmacodynamics were evaluated by monitoring blood glucose and blood insulin levels; acetaminophen was co-administered with glucose to monitor gastric emptying. The study included a single dose treatment of approximately 11 week old ZDF rats (5 rats per treatment group). The OGTT was conducted as a 1 g/kg bolus dose of a glucose solution administered by oral gavage. Acetaminophen was administered, at a dose of 100 mg/kg. For timing purposes the OGTT was designated as time=0 minutes. Dose administration for saline, GLP-1, or exendin-4 was at −10 minutes relative to the OGTT. Blood glucose was evaluated at the following time points: pre-OGTT (twice) and 5, 15, 30, 45, 60, 75, 90, 120, 180 and 240 minutes post-OGTT. Blood insulin was evaluated at the following time points: pre-OGTT and 5, 15, 30, 45, 60, 75, 90, 120 and 180 minutes post-OGTT. APAP blood level was evaluated at the following time points: pre-OGTT and 15, 30, 45, 60, 75, 90, 120, and 180 minutes post-OGTT.

### Pharmacodynamic Results

Table 44 shows glucose level pharmacodynamic results. Peak levels (Cₘₐₓ) for glucose were 70% of control in animals administered GLP-1; similarly the AUC for glucose for the time frame of 0 to 60 (AUC₀₋₆₀) minutes post-OGTT was 70% of control. Administration of Exendin-4 had minimal effects on Cₘₐₓ (ranging from 86% to 99% of control) or AUC₀₋₆₀ (ranging from 91% to 103% of control). There was no clear dose-response for exendin-4.

TABLE 44
Glucose Level Pharmacodynamic Results for GRPs: GLP-1 and Exendin-4

| Formulation | Tₘₐₓ | Cₘₐₓ | % of | AUC₀₋₆₀ | % of | AUC₀₋₂₄₀ | % of |
|---|---|---|---|---|---|---|---|
| (Group #) | (min) | (mg/dL) | Control | (min*mg/dL) | Control | (min*mg/dL) | Control |
| Saline Control | 45 | 331 | — | 17050 | | 54280 | — |
| (#1) | | | | | | | |
| 100 ug/kg GLP- | 180 | 231 | 70 | 11973 | 70 | 51520 | 95 |
| 1 (EDTA-based) | | | | | | | |
| (#2) | | | | | | | |
| 2 ug/kg Ex-4 | 45 | 328 | 99 | 17533 | 103 | 58580 | 108 |
| (EDTA-based) | | | | | | | |
| (#3) | | | | | | | |
| 10 ug/kg Ex-4 | 75 | 328 | 99 | 17488 | 103 | 61408 | 113 |
| (EDTA-based) | | | | | | | |
| (#4) | | | | | | | |
| 20 ug/kg Ex-4 | 60 | 321 | 97 | 16063 | 94 | 66680 | 123 |
| (EDTA-based) | | | | | | | |
| (#5) | | | | | | | |
| 2 ug/kg Ex-4 | 30 | 289 | 87 | 15580 | 91 | 51805 | 95 |
| (PDF-based) | | | | | | | |
| (#6) | | | | | | | |
| 10 ug/kg Ex-4 | 45 | 286 | 86 | 15818 | 93 | 57503 | 106 |
| (PDF-based) | | | | | | | |
| (#7) | | | | | | | |

Table 45 shows the glucose level pharmacodynamic results after correction for endogenious glucose. Peak levels (Cₘₐₓ) for glucose were 51% of control in animals administered GLP-1; similarly the AUC for glucose for the time frame of 0 to 60 (AUC₀₋₆₀) minutes post-OGTT was 46% of control, and AUC₀₋₂₄₀ (0 to 240 minutes post-OGTT) was 88% of control. The administration of exendin-4 in the EDTA-based or PDF-based formulations had moderate effects on Cₘₐₓ(ranging from 73% to 87% of control) or AUC₀₋₆₀ (ranging from 91% to 103% of control). Minimal to no effect was noted for AUC₀₋₂₄₀, ranging from 77% to 125% of control.

TABLE 45
Glucose Level (Corrected for Endogenous Glucose)
Pharmacodynamic Results for GRPs: GLP-1 and Exendin-4.

| Formulation | Tₘₐₓ | Cₘₐₓ | % of | AUC₀₋₆₀ | % of | AUC₀₋₂₄₀ | % of |
|---|---|---|---|---|---|---|---|
| (Group #) | (min) | (mg/dL) | Control | (min*mg/dL) | Control | (min*mg/dL) | Control |
| Saline Control | 45 | 206 | — | 9550 | — | 24280 | — |
| (#1) | | | | | | | |
| 100 ug/kg | 180 | 105 | 51 | 4413 | 46 | 21280 | 88 |
| GLP-1 (EDTA- | | | | | | | |
| based) (#2) | | | | | | | |
| 2 ug/kg Ex-4 | 45 | 174 | 84 | 8293 | 87 | 21620 | 89 |
| (EDTA-based) | | | | | | | |
| (#3) | | | | | | | |
| 10 ug/kg Ex-4 | 75 | 179 | 87 | 8548 | 90 | 25647.5 | 106 |
| (EDTA-based) | | | | | | | |
| (#4) | | | | | | | |
| 20 ug/kg Ex-4 | 60 | 170 | 83 | 7003 | 73 | 30440 | 125 |
| (EDTA-based) | | | | | | | |
| (#5) | | | | | | | |
| 2 ug/kg Ex-4 | 30 | 151 | 73 | 7300 | 76 | 18685 | 77 |
| (PDF-based) | | | | | | | |
| (#6) | | | | | | | |
| 10 ug/kg Ex-4 | 45 | 155 | 75 | 7958 | 83 | 26063 | 107 |
| (PDF-based) | | | | | | | |
| (#7) | | | | | | | |

Insulin response (corrected for endogenous insulin) results showed that post-OGTT insulin levels in control animals were similar or slightly lower, as compared to pre-dose values. Nasal administration of GLP-1 at 100 μg/kg was associated with an up to 2-fold increase in insulin at 5 to 45 minutes post-OGTT. Insulin levels following dosing of exendin-4 at 2, 10, and 20 μg/kg (EDTA-based formulation) demonstrated a dose-dependent increase in insulin with peak levels being approximately 0.7-fold, 1.5-fold, and 4-fold, respectively, above pre-dose values for each group. The response was limited to approximately 45-minutes post-OGTT. Following nasal administration of exendin-4 in the PDF-based formulation at 2 or 10 μg/kg doses, peak insulin levels were 1.5-fold or 2.7-fold, respectively, above pre-dose. The response was limited to approximately 45 minutes post-OGTT.

### Gastric Emptying Results

Table 46 shows the acetaminophen (gastric emptying) results. In controls, peak acetaminophen levels (Cₘₐₓ) of 73 ng/mL occurred at 30 minutes post-dose (Tₘₐₓ). In animals administered GLP-1, a slightly lower Cₘₐₓ (66 ng/mL) was noted; however, Tₘₐₓ was longer suggesting a delay in gastric emptying at this dose level. Administration of exendin-4 in the EDTA-based formulation at 2 or 10 μg/kg or PDF-based formulations at 2 ug/kg was similar to control for Tₘₐₓ (30 to 45 minutes) and Cₘₐₓ(approximately 70 to 73 ng/mL). Administration of exendin-4 in the EDTA-based formulation at 20 μg/kg or PDF-based formulations at 10 μg/kg demonstrate a decreased in Cₘₐₓ (approximately 51 to 54 ng/mL), and at least for the 20 μg/kg (EDTA-based formulation) a slightly prolonged exposure profile. The results suggested that gastric emptying was impacted at these dose levels.

TABLE 46
Summary of GRP Acetaminophen (Gastric Emptying) Results

| | Tₘₐₓ | Cₘₐₓ | % of |
|---|---|---|---|
| Formulation (Group #) | (min) | (mg/dL) | Control |
| Saline Control (#1) | 30 | 73.1 | — |
| 100 μg/kg GLP-1 (EDTA-based) (#2) | 60 | 66.1 | 90 |
| 2 μg/kg Ex-4 (EDTA-based) (#3) | 45 | 69.9 | 96 |
| 10 μg/kg Ex-4 (EDTA-based) (#4) | 30 | 71.9 | 98 |
| 20 μg/kg Ex-4 (EDTA-based) (#5) | 45 | 51.1 | 70 |
| 2 μg/kg Ex-4 (PDF-based) (#6) | 45 | 72.9 | 100 |
| 10 μg/kg Ex-4 (PDF-based) (#7) | 45 | 54.3 | 74 |

### Summary

Based on the pharmacologic effects of GLP-1 and exendin-4 with regard to glucose-dependent stimulation of insulin release, the described EDTA-based and PDF-based intranasal formulations effectively delivered active drug to systemic targets. The stimulation for insulin release was dose-dependent.

Modulation of peak levels (Cₘₐₓ) or exposure (AUC) for blood glucose was shown in animals administered GLP-1 or exendin-4, as compared to controls. Stimulation of insulin release and modulation of gastric emptying, by GLP-1 and exendin-4 are the likely pharmacologic basis for modulation of the blood glucose profile following an oral glucose load.

Inhibition of gastric emptying is a known pharmacologic action of GLP-1 and exendin-4. The change in time to peak concentration (Tₘₐₓ) or peak concentration (Cₘₐₓ) for acetaminophen was consistent with GLP-1- and exendin-4-induced pharmacology. These data support the efficacy of the GLP-1 and exendin-4 formulations described in these assays for use in the treatment of metabolic disorders such as obesity and diabetes.

Although the foregoing invention has been described in detail by way of example for purposes of clarity of understanding, it is apparent to the artisan that certain changes and modifications are comprehended by the disclosure and may be practiced without undue experimentation within the scope of the appended claims, which are presented by way of illustration, not limitation.

1.-42. (canceled)

43. An aqueous pharmaceutical formulation for intranasal delivery comprising a therapeutically effective amount of exendin-4 or an exendin-4 agonist analog, a permeation-enhancing solubilizing agent, a permeation-enhancing cation chelator, a buffer and optionally a permeation-enhancing viscosity enhancing agent, wherein
a. the solubilizing agent is selected from at least one of the group consisting of hydroxypropyl-β-cyclodextrin, sulfobutylether-β-cyclodextrin, dimethyl-β-cyclodextrin, methyl-β-cyclodextrin and Cremophor EL, and
b. the viscosity enhancer is selected from at least one of the group consisting of gelatin, methylcellulose and hydroxypropylmethylcellulose, and wherein
c. the formulation provides at least 5% permeation of exendin-4 in an in vitro tissue permeation assay, has a viscosity up to 150 cps, has a pH from 2 to 8 and is stable at least two weeks at 5° C.

44. The formulation of claim 43, wherein the solubilizing agent is methyl-β-cyclodextrin.

45. The formulation of claim 44, wherein methyl-β-cyclodextrin is present at a concentration of up to 90 mg/ml.

46. The formulation of claim 45, wherein methyl-β-cyclodextrin is present at 80 mg/ml.

47. The formulation of claim 43, wherein the chelator is selected from at least one of the group consisting of ethylene diamine tetraacetic acid and ethylene glycol tetraacetic acid.

48. The formulation of claim 47, wherein the chelator is present at a concentration of up to 10 mg/ml.

49. The formulation of claim 48, wherein the chelator is present at 5 mg/ml.

50. The formulation of claim 43, wherein the solubilizing agent concentration is 80 mg/ml and the chelator concentration is 5 mg/ml.

51. The formulation of claim 43, wherein the buffer is a mono-ionogenic buffer.

52. The formulation of claim 51, wherein the mono-ionogenic buffer is selected from at least one of the group consisting of acetate, arginine and lactate.

53. The formulation of claim 52, wherein the mono-ionogenic buffer is arginine.

54. The formulation of claim 53, wherein arginine is present at a concentration greater than or equal to 2.8 mM.

55. The formulation of claim 43, wherein the pH is from 4.7 to 5.5.

56. The formulation of claim 43, wherein a preservative is not present.

57. The formulation of claim 43, wherein the viscosity is from 1.5 to 10.0 cps.

58. The formulation of claim 43, wherein the formulation is stable for at least 4 weeks at 5° C.

59. A method of treating a subject in need or desirous thereof, comprising administering the aqueous pharmaceutical formulation of claim 43 to the subject by intranasal delivery to treat a metabolic disease selected from the group consisting of hyperglycemia, insulin dependent diabetes mellitus, gestational diabetes, non insulin-dependent diabetes mellitus, obesity or dyslipidemia or to treat a condition benefited by suppressing appetite, increasing satiety, promoting weight loss, decreasing food intake, slowing gastric emptying, lowering plasma glucose or promoting insulin secretion.
